(19) United States
(12) Patent Application Publication
(10) Pub. No.: US 2024/0301498 A1
(43) Pub. Date: Sep. 12, 2024

(54) DIAGNOSTIC METHOD FOR CANCER EVOLUTION PREDICTION, AND USES THEREOF

(71) Applicants: INSTITUT NATIONAL DE LA SANTE ET DE LA RECHERCHE MEDICALE, Paris (FR); UNIVERSITÉ PAUL SABATIER TOULOUSE III, Toulouse (FR); INSTITUT CLAUDIUS REGAUD, Toulouse (FR)

(72) Inventor: Ataaillah BENHADDOU, Toulouse (FR)

(21) Appl. No.: 18/257,383

(22) PCT Filed: Dec. 13, 2021
(86) PCT No.: PCT/EP2021/085491

§ 371 Date: Jun. 14, 2023

(30) Foreign Application Priority Data
Dec. 14, 2020 (EP) 20306558.6

(51) Int. Cl. C12Q 1/6886; G16B 40/00; G16H 50/30

(52) U.S. Cl. CPC C12Q 1/6886; G16B 40/00; G16H 50/30; C12Q 2600/106; C12Q 2600/118; C12Q 2600/156

(57) ABSTRACT

The present invention relates to the field of cancer diagnostic methods for classifying cancer patients using a measure of the distribution of markers of DNA structure alterations within tumor genomic DNA sequence. The inventors have observed, in sarcomas, such as leiomyosarcoma, that the distribution of breakpoints within a set of selected transcription-associated chromosomal instability elements and a set of selected replication-associated chromosomal instability elements can be used to classify patients in groups according to a level of genome instability, which may be representative of a cancer evolution, a risk of occurrence of metastasis, a survival rate, or a sensibility to a cancer treatment. The present invention also relates to machine learning and trained classifiers for classifying patients having a cancer based upon a distribution of genomic DNA structure alterations or variations.

### FIELD

The present disclosure relates to the field of cancer diagnostic methods. Further, the present disclosure relates to diagnostic methods for classifying cancers and patients having such cancers, using distribution of markers of DNA structure alterations. Also, the present disclosure relates to diagnostic methods for predicting an evolution of a cancer, and methods for treating patients having such cancer based upon the prediction results. The present disclosure also relates to machine learning and trained classifiers for classifying patients having a cancer based upon a distribution of genomic DNA structure alterations or variations.

### BACKGROUND

In 2018, the global cancer burden was estimated to have risen to 18.1 million new cases and to be the cause of 9.6 million deaths. Worldwide, the total number of people who are alive within 5 years of a cancer diagnosis is estimated to be 43.8 million (IARC, press release 263, 12 Sep. 2018). Cancer remains therefore one of the deadliest threats to human health.

Most of cancer belongs to one of the following cancer types: carcinomas, sarcomas, melanomas, lymphomas, and leukemias. Carcinomas are cancer from the skin, lungs, breasts, pancreas, and other organs and glands. Lymphomas are cancers of lymphocytes. Leukemias are cancers of the blood. Sarcomas are cancers of bones, muscles, fat tissues, blood vessels, cartilage, or other soft or connective tissues of the body. Melanomas are cancers that arise from cells that make the pigment in skin. Many cancers tend to grow and spread through metastasis which is a process by which cancer cells travel through the lymphatic or blood vessels to form new tumors, usually named secondary tumors (as opposed to the primary tumors from which they originate), in other parts of the body. The presence of metastasis is a marker of aggravation of a cancer disease affecting greatly the survival of the concerned patient.

Although there have been significant advances in the medical treatment of certain cancers, the overall 5-year survival rate for all cancers has improved only by about 10% in the past 20 years. Despite the significant advancement in the treatment of cancer, improved diagnostic methods are still being sought.

Sarcoma is a rare and complex cancer. It makes up about 1% of all adult cancers, but accounts for about 20% of cancers diagnosed in childhood. There are more than 80 different subtypes of sarcoma, many of which have distinct clinical characteristics with unique natural history and tumor biology. Most common types of sarcoma in adults are undifferentiated pleomorphic sarcoma, liposarcoma, and leiomyosarcoma.

Leiomyosarcoma (LMS), showing smooth muscle cell (SMC) differentiation, is one of the most frequent Soft Tissue Sarcoma histotypes (STS, a group of a hundred different malignancies developed from connective tissue cells). However, LMS remains a rare tumor, accounting for around 600 cases in France each year (Blay et al., Annals of Oncology: Official Journal of the European Society for Medical Oncology 28 (11): 2852-59, 2017). Like other STS, LMS can occur at any anatomical site, with three main locations (limbs, retroperitoneum, and uterus). The keystone of the treatment of patients with localized LMS, is based on a large surgical resection, aimed to get tumor free margins. LMS remains though one of the most aggressive STS subtypes, up to 50% of patients having metastatic relapse. In this case, they are mainly incurable diseases, with a median survival of 12 months (Judson et al., The Lancet. Oncology 15 (4): 415-23, 2014). Anthracyclines based chemotherapy is the main treatment of metastatic LMS, since neither targeted therapy (van der Graaf et al., The Lancet 379 (9829): 1879-86, 2012), nor immunotherapy (Ben-Ami et al., Cancer 123 (17): 3285-90, 2017) showed major therapeutic effects in LMS so far. LMS oncogenesis is organised around frequent p53 and RB1 pathways alterations (Chibon et al., Nature Medicine 16 (7): 781-87, 2010; Derré et al., Laboratory Investigation; a Journal of Technical Methods and Pathology 81 (2): 211-15, 2001) and a highly rearranged genome with a high number of chromosomal rearrangements leading to many copy number variations (CNV) and breakpoints (BP), associated to poor outcome (Pdrot et al., The American Journal of Pathology 177 (4): 2080-90, 2010).

LMS stratification has long been based on histological measure like FNCLCC grading (Coindre et al., Cancer 91 (10): 1914-26, 2001) and is currently challenged by expression-based signatures (Chibon et al., Nature Medicine 16 (7): 781-87, 2010). NGS has lately demonstrated the capacity to identify clinically actionable genetic variants across the large number of genes (Spencer et al., The Journal of Molecular Diagnostics 15 (5): 623-33, 2013). Current approaches in cancer genomics often uses exome-seq to build mutations catalogue in multiple cancer types through sequencing hundreds of tumor samples in order to find diagnostic, prognostic, and therapeutic targets (Shabani Azim et al., Iranian Journal of Public Health 47 (10): 1453-57, 2018). While this approach remains relevant in the majority of cancer types it becomes rapidly insufficient in highly rearranged cancer types like LMS, where driver gene mutations are very rare (Andersson et al., Cancer Genetics 209 (4): 154-60, 2016) and it is more likely that their rearranged genome is actually the driver force of oncogenesis (Davoli et al., Science 355 (6322): eaaf8399, 2017).

Therefore, there is a need to have a new and efficient methods and tools for LMS stratification, and patients' classification.

Cancer may be defined as the uncontrolled growth of cells due, mainly, to alteration of the genomic DNA of the cells leading to genomic instability (GI). GI is a hallmark of cancer (Negrini, Gorgoulis, et Halazonetis, Nature Reviews Molecular Cell Biology 11 (3): 220-28, 2010). GI may arise as a consequence of deleterious mutation in components of DNA repair pathways or by abnormal high levels of genotoxic stress from cellular processes such as transcription and replication that overwhelm high-fidelity DNA repair (Tubbs et Nussenzweig, Cell 168 (4): 644-56, 2017). Replication stress is a threat to genome stability and has been implicated in tumorigenesis (Gaillard, García-Muse, et Aguilera, Nature Reviews Cancer 15 (5): 276-89, 2015; Macheret et Halazonetis, Annual Review of Pathology: Mechanisms of Disease 10 (1): 425-48, 2015; Técher et al., Nature Reviews Genetics 18 (9): 535-50, 2017). Notably, common fragile sites in cancer colocalize with chromosomal regions particularly prone to breakage following mild replication stress (Debatisse et al., Trends in Genetics 28 (1): 22-32, 2012; Le Tallec et al., Cell Reports 4 (3): 420-28, 2013; Blin et al., Nature Structural & Molecular Biology 26 (1): 58-66, 2019). Transcription also creates conditions for mutations and recombination as well as DNA breaks, either by transcription-associated processes or by its ability to become a barrier to DNA replication (Aguilera, The EMBO Journal 21 (3): 195-201, 2002; Jinks-Robertson et Bhagwat, Annual Review of Genetics 48 (1): 341-59, 2014; Kim et Jinks-Robertson, Nature Reviews Genetics 13 (3): 204-14, 2012; Gaillard, Herrera-Moyano, et Aguilera, Chemical Reviews 113 (11): 8638-61, 2013; Marnef, Cohen, et Legube, Journal of Molecular Biology 429 (9): 1277-88, 2017). Indeed, co-transcription R-loops constitute a barrier for replication fork progression and lead to fork stalling and collapse and is proposed as a major mechanism of GI which implicates transcription-replication collisions (Helmrich, Ballarino, et Tora, Molecular Cell 44 (6): 966-77, 2011; Wilson et al., Genome Research 25 (2): 189-200, 2015; Pentzold et al., Nucleic Acids Research 46 (3): 1280-94, 2018; Madireddy et al., Molecular Cell 64 (2): 388-404, 2016). Finally, obstacles on the template DNA, such as non-B DNA (NBD) structures, DNA repeats or DNA-bound non-histone proteins as well as transcription complexes can impede replication fork progression (Gaillard et Aguilera 2016, Azvolinsky et al., Molecular Cell 34 (6): 722-34, 2009; French, Science 258 (5086): 1362-65, 1992; Deshpande et Newlon, Science 272 (5264): 1030-33, 1996; Gómez-Gonzalez et al., The EMBO Journal 30 (15): 3106-19 2011).

GI has long been suggested to be predictive of bad prognosis, but an accurate measure method remains to be discovered (Ahmad, Ahmed, et Venkitaraman, Clinical Oncology 30 (12): 751-55, 2018). Furthermore, the current lack of a measure that captures the dynamic nature of the GI limits possibility to leverage it for prognostic and therapeutic purposes (Sansregret, Vanhaesebroeck, et Swanton, Nature Reviews Clinical Oncology 15 (3): 139-50, 2018) in a clinical setting.

Therefore, there is a need to have tools allowing to evaluate whether transcription complexes, as well as NBD and DNA repeats play any role in cancer GI, such as for example LMS GI.

Most of the methods and biomarkers used for detecting cancer and stratifying patients according to specific criteria, for example cancer aggressiveness or responsiveness to cancer treatment, rely upon the identification and quantification of specific events, for example mutations, breakpoints, or deletions, in specific regions of the genomic DNA of the patients, e.g., specific genes.

There is a need to have statistical biomarkers which levels are immediately transferable to a level of confidence.

There is also a need to have methods, tool, and biomarkers allowing measuring of the propensity of a given DNA elements to break more than expected under random breakage model.

There is also a need to have methods, tools, and biomarkers allowing measuring prognostic of metastatic risk in cancer, such as for example LMS.

Presence of metastasis or level of genome instability may affect sensibly the outcome of a cancer of a patient, sensibility of the cancer to a cancer treatment, cancer patient survival, or even the choice of a treatment to be proposed to a patient.

The current lack of appropriate tools to capture the dynamic nature of the GI limits the ability to leverage GI for prognostic and therapeutic purposes (Sansregret, Vanhaesebroeck, et Swanton, 2018). Furthermore, the lack of tools allowing prediction of occurrence of metastasis or prediction of sensibility of a cancer to a cancer treatment is a hurdle preventing to provide the most appropriate treatment to a cancer patient.

Therefore, there is a need to have a biomarker allowing to classify cancer patient in a group representative of an outcome or an evolution of a cancer disease. As understood herein, a “biomarker” is a “characteristic that is objectively measured and evaluated as an indicator of normal biological processes, pathogenic processes, such as a cancer, or pharmacologic responses to a therapeutic intervention, such as a cancer treatment” (Biomarkers Definitions Working Group., Clin Pharmacol Ther. 2001 March; 69(3):89-95). A biomarker may be a biological parameter, such as an amount of a protein, a metabolite, a DNA sequence, a marker of DNA structure alteration, or a statistical representation of such biological parameter.

There is a need to have methods, tools, and biomarkers allowing to classify a cancer patient according to cancer's evolution, such as evolution towards metastasis, evolution in response to a cancer treatment, or evolution in terms of survival rate.

There is a need to have methods, tools, and biomarkers allowing to classify a cancer patient, as for example in a group representative of a sensibility of the cancer of the patient to a cancer treatment.

There is a need to have methods, tools, and biomarkers allowing to classify a cancer patient in a group representative of a risk of occurrence of cancer metastasis.

There is also a need to have methods, tools, and biomarkers allowing to predict a risk of occurrence of metastasis in a patient having a cancer.

There is a need to have methods, tools, and biomarkers allowing to predict the sensibility of a cancer from a cancer patient to a cancer treatment.

There is also a need to have methods, tools, and biomarkers allowing to predict an outcome, survival or mortality, for a predefined period of time for a cancer patient.

There is also a need to have a diagnostic and treatment method of a cancer patient by which a cancer treatment is administered to the cancer patient according to a result of the diagnostic method.

There is also a need to have a method and a trained classifier allowing to obtain threshold level of a predefined biomarker allowing to classify a cancer patient in a group representative of an evolution of the cancer of the patient.

The present invention has for object to satisfy all or part of these needs.

### SUMMARY

The present invention relates to a method for classifying a patient having a cancer in a group representative of an evolution of said cancer, especially representative of a risk of metastasis of said cancer, especially for leiomyosarcoma, the method using:
i) a tumor genomic DNA sequence obtained by sequencing a tumor genomic DNA obtained from said patient,
ii) at least one set of preselected DNA elements, each DNA element having a genomic position, said set of DNA elements having a length L in base pairs,
iii) a preselected genomic DNA sequence of reference having a length l in base pairs, and
iv) at least one reference threshold separating two groups of patients, each group being representative of a different evolution of said cancer,
   said method comprising at least the steps of,
a) identifying, determining genomic positions, and quantifying a total quantity n of a predefined marker of DNA structure alteration within said tumor genomic DNA sequence of the tumor genomic DNA from said patient, by comparing said tumor genomic DNA sequence with said preselected genomic DNA sequence of reference,
b) associating, to the set of preselected DNA elements, a quantity N of the predefined marker of DNA structure alteration, from the total quantity n, according to the genomic positions of the DNA elements of the set of preselected DNA elements and to the genomic positions of said predefined markers of DNA structure alteration obtained at step a),
c) computing a test score relative to a quantity N of predefined markers of DNA structure alteration associated with the length L of said set of preselected DNA elements, said test score being based on a comparison between said quantity N of predefined markers of DNA structure alteration and a reference level defined by a random model relative to the total quantity n of the predefined marker of DNA structure alteration over the length l of the preselected genomic DNA sequence of reference, and
d) comparing said test score to said at least one reference threshold in order to classify said patient.

Surprisingly, and as disclosed in the Examples Section, the inventors have observed that a marker of DNA structure alteration, such as breakpoints or copy number variations, may be used with a trained classifier to classify cancer patients in groups representative of a level of genome instability. A patient may be classified as low, medium or high. The level of genome instability may be used to predict evolution of the cancer of those patients, risk of occurrence of metastasis or sensibility to a cancer treatment.

Also, it was observed that the distribution of a quantified marker of DNA structure alteration, such as breakpoints, within the length of some DNA elements, such as indicated below, may be used to compute a score allowing to be compared to thresholds and to classify a cancer patient in a group representative of a level of genome instability, which may be representative of an evolution of the cancer of the patient, such as a risk of occurrence of metastasis or a sensibility to a cancer treatment.

As shown in the Examples section, the inventors have observed that a statistical representation of the distribution of a quantified marker of DNA structure and/or function alteration was useable as a biomarker for stratifying cancer patient.

As shown in the Examples section, the Hscore developed by the inventors as a statistical representation of the distribution of a quantified marker of DNA structure and/or function alteration is methodologically and conceptually different from the Tumor Mutation Burden (TMB) which is the number of somatic mutations per megabase of interrogated genomic sequence. There is no correlation between TMB and Hscore.

In one exemplary embodiment, the method further comprises the use of a non-tumor genomic DNA sequence obtained by sequencing a genomic DNA obtained from non-cancer cells of said patient.

In one exemplary embodiment, step a) may comprise aligning and comparing the tumor genomic DNA sequence, the non-tumor genomic DNA, and the preselected genomic DNA sequence of reference.

In one exemplary embodiment, the predefined marker may be selected in the group comprising breakpoint, insertion, deletion, mutation, duplication, inversion, translocation, complex genomic rearrangements, copy number variation, telomeric allelic imbalance, large-scale state transitions, and loss of heterozygosity.

In one exemplary embodiment, said DNA elements are selected in a group comprising direct repeats (DR), short tandem repeats (STR), mirror repeats (MR), inverted repeats (IR), Z DNA, simple repeats (SR), microsatellites (MS), low complexities (LC), R-loops forming sequences (RLFS), G-quadruplexes (GQ), CpG islands (CpGi), cis-regulated modules (CRM), self-chains segments self-aligned (SCS-S), DNase I hypersensitivity site (DHS) promoters (DHS_prom), DNase I hypersensitivity site (DHS) enhancers (DHS_enh), DNase I hypersensitivity site (DHS) dyadic regulatory elements (DHS_dyadic), and DNase I hypersensitivity site (DHS) of type rest (DHS_rest), transcription factor binding sites, and mixtures thereof.

In another exemplary embodiment, said DNA elements are selected in a group comprising direct repeats (DR), short tandem repeats (STR), mirror repeats (MR), inverted repeats (IR), Z DNA, simple repeats (SR), microsatellites (MS), low complexities (LC), R-loops forming sequences (RLFS), G-quadruplexes (GQ), CpG islands (CpGi), cis-regulated modules (CRM), self-chains segments self-aligned (SCS-S), DNase I hypersensitivity site (DHS) promoters (DHS_prom), DNase I hypersensitivity site (DHS) enhancers (DHS_enh), and DNase I hypersensitivity site (DHS) dyadic regulatory elements (DHS_dyadic), transcription factor binding sites, and mixtures thereof.

In another exemplary embodiment, DNA elements which may be used within the present disclosure may be selected among direct repeats (DR), short tandem repeats (STR), mirror repeats (MR), inverted repeats (IR), Z DNA, simple repeats (SR), microsatellites (MS), low complexities (LC), R-loops forming sequences (RLFS), G-quadruplexes (GQ), CpG islands (CpGi), self-chains segments self-aligned (SCS-S), transcription factor binding sites, and mixtures thereof.

In one exemplary embodiment, said set of preselected of DNA elements consists in replication-associated chromosomal instability elements, and especially consists in direct repeats (DR), short tandem repeats (STR), mirror repeats (MR), inverted repeats (IR), Z DNA, simple repeats (SR), microsatellites (MS), and low complexities (LC), and mixtures thereof.

In one exemplary embodiment, said set of preselected of DNA elements consists in transcription-associated chromosomal instability elements, and especially consists of R-loops forming sequences (RLFS), G-quadruplexes (GQ), CpG islands (CpGi), cis-regulated modules (CRM), self-chains segments self-aligned (SCS-S), DNase I hypersensitivity site (DHS) promoters (DHS_prom), DNase I hypersensitivity site (DHS) enhancers (DHS_enh), DNase I hypersensitivity site (DHS) dyadic regulatory elements (DHS_dyadic), and DNase I hypersensitivity site (DHS) of type rest (DHS_rest), transcription factor binding sites, and mixtures thereof.

In another exemplary embodiment, said set of preselected of DNA elements consists in transcription-associated chromosomal instability elements, and especially consists of R-loops forming sequences (RLFS), G-quadruplexes (GQ), CpG islands (CpGi), cis-regulated modules (CRM), self-chains segments self-aligned (SCS-S), DNase I hypersensitivity site (DHS) promoters (DHS_prom), DNase I hypersensitivity site (DHS) enhancers (DHS_enh), and DNase I hypersensitivity site (DHS) dyadic regulatory elements (DHS_dyadic), transcription factor binding sites, and mixtures thereof.

In one exemplary embodiment, a set of preselected DNA elements may be a set of transcription-associated chromosomal instability elements. Transcription-associated chromosomal instability elements may be selected, for example, among R-loops forming sequences (RLFS), G-quadruplexes (GQ), CpG islands (CpGi), self-chains segments self-aligned (SCS-S), transcription factor binding sites, and mixtures thereof.

In one exemplary embodiment, the predefined marker of DNA structure alteration is a breakpoint, and a quantity of the predefined marker of DNA structure alteration is associated to each DNA element as a quantity N₁, N₂, N₃, . . . of the predefined marker of DNA structure alteration, from the total quantity n, said association being according to the genomic positions of said DNA element and to the genomic positions of the predefined markers of DNA structure alteration.

In this case, the method according to the invention may comprise after step b) and before step c), a step of summing the quantities N₁, N₂, N₃, . . . , to obtain a quantity N of predefined markers of DNA structure alteration associated with the set of preselected DNA elements.

In one exemplary embodiment, in step c), the test score uses a probability p of having said quantity N of predefined markers of DNA alteration within the length L of the set of preselected DNA elements, and the probability pᵤ is a ratio of the total quantity n of identified and quantified predefined markers of DNA structure alteration to the length l of the preselected genomic DNA sequence of reference.

In one exemplary embodiment, the probability pᵤ may be relative to a uniform distribution.

In one exemplary embodiment, the random model used in step c) may be a random breakage model, especially using a binomial distribution.

In one exemplary embodiment, in step c), the probability p of having said quantity of predefined markers of DNA structure alteration associated with the length L of said set of preselected DNA elements is a one-sided p-value wherein only the values superior to said reference level are taken into account, and said test score is computed as a logarithmic transformation of said p-value.

In one exemplary embodiment, said tumor genomic DNA sequence of said patient is obtained by a sequencing method selected among whole genomic DNA sequencing and DNA targeted sequencing.

In another embodiment, the present disclosure relates to a diagnostic method for predicting an evolution of a cancer in a patient having said cancer, said method:
using at least a tumor genomic DNA sequence obtained by sequencing a tumor genomic DNA obtained from said patient, and
comprising at least the step of submitting said tumor genomic DNA sequence to the method as disclosed herein for classifying a cancer patient in a group representative of an evolution of said cancer, said classification being predictive of an evolution of said cancer of said patient.

According to one exemplary embodiment, said diagnostic method is for predicting (i) a risk of metastasis of said cancer, (ii) a sensibility of said cancer to a cancer treatment, or (iii) a risk of mortality of said patient to said cancer.

According to one exemplary embodiment, said diagnostic method is for predicting a risk of metastasis of said cancer or a sensibility of said cancer to a cancer treatment and said set of preselected of DNA elements consists in transcription-associated chromosomal instability elements, in replication-associated chromosomal instability elements, or in a combination of transcription-associated chromosomal instability elements and replication-associated chromosomal instability elements.

According to one exemplary embodiment, the present disclosure relates to a diagnostic method for identifying a genome instability mechanism of a cancer in a patient having said cancer, said method:
using at least a tumor genomic DNA sequence obtained by sequencing a tumor genomic DNA obtained from said patient, and
comprising at least the step of submitting said tumor genomic DNA sequence to the method according to the invention for classifying a cancer patient in a group representative of a genome instability mechanism being either a replication-associated chromosomal instability or a transcription-associated chromosomal instability.

According to one exemplary embodiment, said diagnostic method is for determining a genome instability of genome of cancer cells of said cancer of said patient and said set of preselected DNA elements consists in transcription-associated chromosomal instability elements or in replication-associated chromosomal instability elements or in a combination of transcription-associated chromosomal instability elements and replication-associated chromosomal instability elements.

In another embodiment, the present disclosure relates to a method for selecting a treatment of a cancer for a patient having said cancer and for treating said patient, said method:
using at least a tumor genomic DNA sequence obtained by sequencing a tumor genomic DNA obtained from said patient, and
the method comprising at least the steps of:
   submitting said at least tumor genomic DNA sequence to the method as disclosed herein for classifying said cancer of said patient in a group associating said cancer with at least one cancer treatment, and
   treating said patient with said at least one cancer treatment associated with said group.

In another embodiment, the present disclosure relates to a method for predicting a sensibility of a cancer in a patient having said cancer to a cancer treatment and for treating said patient, said method:
using at least a tumor genomic DNA sequence obtained by sequencing a tumor genomic DNA obtained from said patient, and
the method comprising at least the steps of:
   submitting said at least tumor genomic DNA sequence to the method as disclosed herein for classifying said cancer of said patient in a group representative of a sensibility of said cancer to a cancer treatment, said classification being predictive of a sensibility of said cancer of said patient to said cancer treatment, and
   treating said patient with said cancer treatment.

In one exemplary embodiment, said cancer may be selected among leukemias, lymphomas, carcinomas, melanomas, and sarcomas.

In one exemplary embodiment, said sarcoma may be an undifferentiated pleomorphic sarcoma, a liposarcoma, a rhabdomyosarcoma, an angiosarcoma from blood vessels, a malignant peripheral nerve sheath tumor (MPNST or PNST), a gastrointestinal stromal tumor sarcoma (GIST), a synovial sarcoma, a dermatofibrosarcoma, a fibrohistiocytic sarcoma, a myxofibrosarcoma, a Kaposi sarcoma, a chondro-osseous sarcoma, a leiomyosarcoma, or any other subtype of sarcoma.

In another embodiment, the present disclosure relates to a method for determining threshold(s) representative of an evolution of a cancer, especially representative of a risk of metastasis for patients having said cancer, especially for leiomyosarcoma, especially to be used in the classification method as disclosed herein, the method using:
i) a classifier,
ii) a plurality of tumor genomic DNA sequences obtained by sequencing a plurality of tumor genomic DNA obtained from a plurality of patients having said cancer,
iii) at least one set of preselected DNA elements, each DNA element having a genomic position, said preselected set of DNA elements having a length L in base pairs,
iv) a preselected genomic DNA sequence of reference having a length l in base pairs, and
v) a plurality of scores relative to a quantity of a predefined marker of DNA structure alteration associated with the length L of said set of preselected DNA elements,
   said predefined markers of DNA structure alteration being previously quantified and their genomic positions identified within each tumor genomic DNA sequence of the tumor genomic DNA from each patient, by comparing said tumor genomic DNA sequence with said preselected genomic DNA sequence of reference, to obtain for each tumor genomic DNA sequence a total quantity n of predefined markers of DNA structure alteration for each genomic DNA sequence,
   said scores being based on a comparison between each quantity of predefined markers of DNA structure alteration and a reference level defined by a random model relative to each total quantity n of the predefined marker of DNA structure alteration over the length l of the preselected genomic DNA sequence of reference,
      said method comprising at least the steps of:
a) choosing, based on said scores, several predefined thresholds relative to said quantified predefined marker associated with the length L of said set of preselected DNA elements, each of said predefined thresholds separating two groups representative of a different evolution of said cancer,
b) for each of said predefined thresholds, computing with the classifier, by using an estimation method, a comparison value between two groups defined by the corresponding threshold, and
c) determining at least one reference threshold as separating two groups representative of a different evolution of said cancer by choosing a predefined threshold that has given an extremum value of said comparison values computed at step b).

In one exemplary embodiment, the estimation method used in step b) is the Kaplan-Meier test, said comparison value is a p-value, and said extremum value is a minimum.

In one exemplary embodiment, the method as disclosed herein may be applied on groups of at least 3 patients having said cancer, better at least 7 patients.

In one exemplary embodiment, two reference thresholds may be determined to define three different groups representative of a different evolution of said cancer.

In another embodiment, the present disclosure relates to a method for selecting a DNA element as a biomarker of an evolution of a cancer in a patient having said cancer, the method using:
i) a tumor genomic DNA sequence obtained by sequencing a tumor genomic DNA obtained from said patient,
ii) a plurality of preselected DNA elements, each DNA element having a genomic position, a length le in base pairs,
iii) a preselected genomic DNA sequence of reference having a length l in base pairs, and
iv) a preselected threshold or a set of preselected thresholds for selecting said DNA element to be used as a biomarker,
   said method comprising at least the steps of:
a) identifying, determining genomic positions, and quantifying a total quantity n of a predefined marker of DNA structure alteration within said tumor genomic DNA sequence of the tumor genomic DNA from said patient, by comparing said tumor genomic DNA sequence with said preselected genomic DNA sequence of reference,
b) associating, to each DNA element of the plurality of DNA elements, a quantity Ne of the predefined marker of DNA structure alteration, from the total quantity n, according to the genomic positions of said DNA element and to the genomic positions of said predefined markers of DNA structure alteration obtained at step b),
c) computing for each DNA element a test score relative to said quantity Ne of predefined markers of DNA structure alteration associated with the length le of said DNA element, said test score being based on a comparison between said quantity Ne of predefined markers of DNA structure alteration and a reference level defined by random model relative to the total quantity n of the predefined marker of DNA structure alteration over the length l of the preselected genomic DNA sequence of reference, and
d) comparing said test scores to said preselected threshold or said set of preselected thresholds to select said DNA element as a biomarker of an evolution of a cancer in a patient having said cancer.

In another embodiment, the present disclosure relates to a biomarker of an evolution of a cancer in a patient having said cancer, said biomarker consisting in replication-associated chromosomal instability elements, and especially consisting in direct repeats (DR), short tandem repeats (STR), mirror repeats (MR), inverted repeats (IR), Z DNA, simple repeats (SR), microsatellites (MS), and low complexities (LC).

In another embodiment, the present disclosure relates to a biomarker of an evolution of a cancer in a patient having said cancer, said biomarker consisting in transcription-associated chromosomal instability elements, and especially consisting in R-loops forming sequences (RLFS), G-quadruplexes (GQ), CpG islands (CpGi), cis-regulated modules (CRM), self-chains segments self-aligned (SCS-S), DNase I hypersensitivity site (DHS) promoters (DHS_prom), DNase I hypersensitivity site (DHS) enhancers (DHS enh), DNase I hypersensitivity site (DHS) dyadic regulatory elements (DHS_dyadic), DNase I hypersensitivity site (DHS) of type rest (DHS_rest), transcription factor binding sites, and mixtures thereof.

In another embodiment, the present disclosure relates to a biomarker of an evolution of a cancer in a patient having said cancer, said biomarker consisting in transcription-associated chromosomal instability elements, and especially consisting in R-loops forming sequences (RLFS), G-quadruplexes (GQ), CpG islands (CpGi), cis-regulated modules (CRM), self-chains segments self-aligned (SCS-S), DNase I hypersensitivity site (DHS) promoters (DHS_prom), DNase I hypersensitivity site (DHS) enhancers (DHS_enh), DNase I hypersensitivity site (DHS) dyadic regulatory elements (DHS_dyadic), transcription factor binding sites, and mixtures thereof.

In yet another embodiment, the present disclosure relates to a device for determining threshold(s) representative of an evolution of a cancer, especially a risk of metastasis for patients having said cancer, especially for leiomyosarcoma, comprising a classifier and using:
i) a plurality of tumor genomic DNA sequences obtained by sequencing a plurality of tumor genomic DNA obtained from a plurality of patients having said cancer,
ii) at least one set of preselected DNA elements, each DNA element having a genomic position, said preselected set of DNA elements having a length L in base pairs,
iii) a preselected genomic DNA sequence of reference having a length l in base pairs, and
iv) a plurality of scores relative to a quantity of a predefined marker of DNA structure alteration associated with the length L of said set of preselected DNA elements,
   said predefined markers of DNA structure alteration being previously quantified and their genomic positions identified within each tumor genomic DNA sequence of the tumor genomic DNA from each patient, by comparing said tumor genomic DNA sequence with said preselected genomic DNA sequence of reference, to obtain for each tumor genomic DNA sequence a total quantity n of predefined markers of DNA structure alteration for each genomic DNA sequence,
   said scores being based on a comparison between each quantity of predefined markers of DNA structure alteration and a reference level defined by a random model relative to each total quantity n of the predefined marker of DNA structure alteration over the length l of the preselected genomic DNA sequence of reference,
      said device being configured to at least:
a) choose, based on said scores, several predefined thresholds relative to said quantified predefined marker associated with the length L of said set of preselected DNA elements, each of said predefined thresholds separating two groups representative of a different evolution of said cancer,
b) for each of said predefined thresholds, compute with the classifier, by using an estimation method, a comparison value between two groups defined by the corresponding threshold, and
c) determine at least one reference threshold as separating two groups representative of a different evolution of said cancer by choosing a predefined threshold that has given an extremum value of said comparison values computed at step b).

In another embodiment, the present disclosure relates to a computer program product for classifying a cancer patient in a group representative of the evolution of said cancer, especially representative of a risk of metastasis of said cancer, especially for leiomyosarcoma, using:
i) a tumor genomic DNA sequence obtained by sequencing a tumor genomic DNA obtained from isolated cancer cells of said patient,
ii) at least one set of preselected DNA elements, each DNA element having a genomic position, said preselected set of DNA elements having a length L in base pairs,
iii) a preselected genomic DNA sequence of reference having a length l in base pairs, and
iv) at least one reference threshold separating two groups of patients, each group being representative of a different evolution of said cancer,
   the computer program product comprising a support and stored on this support instructions that can be read by a processor, these instructions being configured to:
a) identifying, determining genomic positions, and quantifying a total quantity n of a predefined marker of DNA structure alteration within said tumor genomic DNA sequence of the tumor genomic DNA from said patient, by comparing said tumor genomic DNA sequence with said preselected genomic DNA sequence of reference,
b) associate, to the set of preselected DNA elements, a quantity of the predefined marker of DNA structure alteration according to the genomic positions of the DNA elements of the set of preselected DNA elements and to the genomic positions of said predefined markers of DNA structure alteration obtained at step a),
c) computing a test score relative to a quantity of predefined markers of DNA structure alteration associated with the length L of said set of preselected DNA elements, said test score being based on a comparison between said quantity of predefined markers of DNA structure alteration and a reference level defined by a random model relative to the total quantity n of the predefined marker of DNA structure alteration over the length l of the preselected genomic DNA sequence of reference, and
d) compare said test score to said at least one reference threshold in order to classify said patient.

### DESCRIPTION OF THE FIGURES

FIG. 1: is a diagram showing steps of methods for determining threshold(s) representative of an evolution of a cancer and for classifying a patient according to the invention.

FIG. 2: shows that leiomyosarcoma (LMS) breakpoints (BP) are log-normal distributed.

FIG. 2A: is a histogram of total BP count per LMS (TBPc) showing that the distribution is skewed. FIG. 2B: is a histogram of natural log of TBPc showing a log-normal distribution of BP over the whole length of genomic DNA.

FIG. 3: shows that regulatory elements, non-B DNA (NBD) and DNA repeats are hotspots.

FIG. 3A: shows the Random Breakage Model (RBM). Hotspots DNA elements are defined as DNA elements containing more BP than expected under RBM and more than surrounding regions. FIG. 3B: shows Hscore profile for DNA elements and sliding upstream and downstream windows for DNA regulatory elements. FIG. 3C. shows Hscore profile for DNA elements and sliding upstream and downstream windows for non-B DNA (NBD). FIG. 3D. shows Hscore profile for DNA elements and sliding upstream and downstream windows for DNA repeats. Horizontal solid line corresponds to Hscore threshold of 3 for hotspotness.

FIG. 4: shows that DNA regulatory elements are exclusively hot inside genes: Hscore for regulatory elements and sliding windows either inside (solid lines) or outside genes (dotted lines). FIG. 4A: Cis-Regulatory Modules (CRM); FIG. 4B: CpG islands (CpGi); FIG. 4C: Dnase Hyper Sensitive sites (DHS) of type promoter (DHS_prom); FIG. 4D: DHS of type Enhancer (DHS_enh); FIG. 4E: DHS of type dyadic (DHS_dyadic); FIG. 4F: DHS of other type (DHS_rest). Horizontal doted line corresponds to Hscore threshold of 3 for hotspotness.

FIG. 5: shows that NBD are hotspots both inside and outside genes: Hscore for NBD and sliding windows either inside (black) or outside (red) genes. FIG. 5A: G-quadruplex (GQ); FIG. 5B: R-loops forming sequences (RFLS); FIG. 5C: Direct Repeats (DR); FIG. 5D: Short Tandem Repeats (STR); FIG. 5E: Mirror Repeats (MR); FIG. 5F: Inverted repeats (IR); FIG. 5G: Z DNA; FIG. 5H: A-phased Repeats (APR). Horizontal solid line corresponds to Hscore threshold of 3 for hotspotness.

FIG. 6: shows that DNA repeats hotspotness relative to genes is dependent upon their type.: Hscore for NBD and sliding windows either inside (solid line) or outside genes (dashed line). FIG. 6A-C: High-copy DNA repeats. A, Simple Repeats. B, MiscoSatellite. C, Low Copy repeats FIG. 6D-E: Viral origin DNA repeats. D, Retro-Transposons. E, Long Terminal Repeats. FIG. 6F-G: Low copy repeats. F, Self-chains self aligned. FIG. 6A: Simple Repeats SR; FIG. 6B: MicroSatellite (MS); FIG. 6C: Low Copy repeats (LC); FIG. 6D: Retro-Transposons (RT); FIG. 6E: Long Terminal Repeats (LTR); FIG. 6F: Self chains self-aligned; FIG. 6G: Self chains regions. Horizontal solid line corresponds to Hscore threshold of 3 for hotspotness.

FIG. 7: shows the stratification of the LMS cohort into Low, Medium, and High levels of TRAC and RACIN using iPART as defined in the present invention. FIG. 7A TRACi (or iTRAC). Top, Kaplan-Meier p-values in function of TRACi (or iTRAC) single threshold dividing the LMS cohort into Low and High groups (black solid line). Bottom horizontal dashed line corresponds to 0.05 arbitrary and commonly accepted significance p-value threshold. Top horizontal dashed line corresponds to the p-value threshold we considered for p-values that will be included in combinatorial double threshold division of the LMS cohort into Low, Medium, and High groups (see Example section for more details). Vertical dashed lines correspond to the best combination of thresholds (tl=0.99, th=2.29) splitting the LMS cohort into Low, High, Medium groups. Bottom, Kaplan-Meier plot for TRACi (or iTRAC) stratified LMS cohort into Low, Medium, and High based on the best combination of thresholds tl=0.99 and th=2.29. FIG. 7B: RACINi (or iRACIN). Top, bottom plots correspond to the same procedure as A. tl and th for TIRSi are 0.74 and 1.30 respectively. tl, Treshold Low. th, Threshold High.

FIG. 8: shows the application of iPART algorithm to the TCGA pan-cancer cohort of 12 cancer of Andor et al, Nature Medicine 22 (1): 105-13, 2016. FIG. 8A: Kaplan-Meier p-values in function of CNVabundance single threshold dividing the TCGA Pan-cancer cohort into Low and High groups (black solid line). Vertical dashed lines correspond to the four thresholds giving the best data segmentation (see text and methods for details) into 5 groups, which correspond from left to right to: 0.14, 0.45, 0.67, 0.86. Horizontal dashed line corresponds to 0.05 arbitrary and commonly accepted significance p-value threshold. FIG. 8B: Kaplan-Meier plot showing the stratification of the TCGA cohort into 5 clinically relevant groups. FIG. 8C. Hazard Ratio (HR) with 95% confidence interval of risk of mortality of each group relative the reference group (G.1). P-values of each HR are shown above each condition. Horizontal dashed line corresponds to Hazard ratio of 1.

FIG. 9: MAGIC outperforms CINSARC and FNCLCC in the stratification of metastatic risk in LMS cohort. Metastasis-free survival curves of LMS cohort stratified by FIG. 9A: MAGIC; FIG. 9B: CINSARC; FIG. 9C: FNCLCC.

FIG. 10: TRACi (or iTRAC) stratifies chemotherapeutic response in LMS but not RACINi (or iRACIN). MFS curves in Low, Medium, and High groups of FIG. 10A: TRACi (or iTRAC) and FIG. 10B: RACINi (or iRACIN) stratified by chemotherapeutic treatment.

FIG. 11: shows on top, middle, bottom plots the best two thresholds stratification of LMS cohort using TBPc, nBPSVinter and nBPSVintra respectively.

FIG. 12: shows the segmentation of the Andor et al Cohort into 2, 3, 4, 5, 6, 7 and 8 groups using iPART. FIG. 12A. Kaplan-Meier p-values of the best data segmentation in function of number of groups. FIG. 12B. Maximal HR of the best data segmentation in function of number of groups.

FIG. 13: represents FIG. 5 of the article Andor et al, 2016.

FIG. 14: represents stratification with ITRACexome and iRACINexome of a chemotherapeutic response in LMS. FIG. 14A. Group of patients without (G.1, left panel) or with (G.2, right panel) iTRACexome biomarker was divided into having chemotherapy (Yes) or not having chemotherapy (No) and differences in metastasis free survival was evaluated using Kaplan-Meier plots. FIG. 14B. Group of patients without (G.1, left panel) or with (G.2, right panel) iRACINexome biomarker was divided into having chemotherapy (Yes) or not having chemotherapy (No) and differences in metastasis free survival was evaluated using Kaplan-Meier plots.

FIG. 15: represents stratification with a combination of iTRACexome and iRACINexome into MAGIC.2 classifier or a single iTRACINexome biomarker of a chemotherapeutic response in LMS. FIG. 15A. patients belonging to Group G.1 in MAGIC are defined as patients which are G.1 in iTRACexome and iRACINexome, otherwise G.2. FIG. 15B iTRACINexome is generated by combining TRAC and RACIN DNA elements present in reference Exome into a single biomarker and the Hscore is computed.

FIG. 16: illustrates that Hscore is different from tumor mutational burden (TMB). FIG. 16A. TMB is a genetic characteristic of tumorous tissue defined as the number of non-inherited mutations per million bases (Mb) of investigated genomic sequence. TMB value does not vary with variation of mutation distribution across the investigated genomic interval. FIG. 16B. Hscore is a statistical characterization of tumorous tissue (the biomarkers), based on the quantification of the likelihood that the observed mutations in a subset of the investigated genomic sequence are not random events. With Hscore, genomic intervals with the same TMB will have different scores reflecting different distributions of mutation. The values of TMB and Hscore are computed for: genomic interval of 100 Mb, and a total of 1000 mutations. For Hscore: biomarker size was 5 Mb and the expected mutation counts is 50 mutations. If the observed mutations is 50 the Hscore is 0.33. Alternatively, if the observed mutations is 75 Hscore is equal to 3.42

FIG. 17: illustrates that Hscore is different from tumor mutational burden (TMB). FIG. 17A. LMS patients Group G.1 not having the biomarker (Hscore <=3) and Group G.2 having the biomarker (Hscore >3): iTRAC (left), iRACIN (middle), iTRACIN (right) do not present any difference in their TMB. FIG. 17B. There is low or no correlation between TMB and our biomarkers iTRAC (left), iRACIN (middle), iTRACIN (wright). TMB was computed as the number of mutations per Mb of reference exome.

### DETAILED DESCRIPTION

### Definitions

The terms used in this specification generally have their ordinary meanings in the art. Certain terms are discussed below, or elsewhere in the present disclosure, to provide additional guidance in describing the products and methods of the presently disclosed subject matter.

The following definitions apply in the context of the present disclosure:

As used in this specification and the appended claims, the singular forms “a”, “an” and “the” include plural referents unless the content clearly dictates otherwise.

The term “about” as used herein refers to the usual error range for the respective value readily known to the skilled person in this technical field. Reference to “about” a value or parameter herein includes (and describes) embodiments that are directed to that value or parameter per se. In some embodiments, the term “about” refers to ±10% of a given value. However, whenever the value in question refers to an indivisible object, such as a nucleotide or other object that would lose its identity once subdivided, then “about” refers to ±1 of the indivisible object.

Within the present description, the term “breakpoint” intends to refer either to a single chromosomal position (position of 1 nucleotide) corresponding to a damage in DNA affecting a single chromosomal position, and may comprise single insertion or single deletion or single mutation, or to any of the genomic positions corresponding to at least two chromosomal positions describing structural alteration affecting genomic intervals of at least 2 bp. Structural genomic or chromosomic alterations which may cause breakpoints may insertion, deletion, mutation, duplication, inversion, translocation, complex genomic rearrangements, copy number variation, telomeric allelic imbalance, large-scale state transitions, and loss of heterozygosity. Within the present disclosure, “copy number variation” is used interchangeably with “copy number alteration” and intends to refer to changes in copy number that have arisen in somatic tissue (for example, just in a tumor) or in germline cells (and are thus in all cells of the organism).

Within the present description, the expression “classifier” intends to mean a learning model with associated learning algorithms that analyze data, used for classification and regression analysis.

Within the present description, the expression “for classifying” intends to mean choosing, for a cancer patient, a group having properties and features representative of an evolution of said cancer.

It is understood that aspects and embodiments of the present disclosure described herein include “comprising”, “consisting of”, “consisting essentially of” and “having” aspects and embodiments. The words “have” and “comprise,” or variations such as “has,” “having,” “comprises,” or “comprising,” will be understood to imply the inclusion of the stated element(s) (such as a composition of matter or a method step) but not the exclusion of any other elements. The term “consisting of” implies the inclusion of the stated element(s), to the exclusion of any additional elements. The term “consisting essentially of” implies the inclusion of the stated elements, and possibly other element(s) where the other element(s) do not materially affect the basic and novel characteristic(s) of the invention. It is understood that the different embodiments of the disclosure using the term “comprising” or equivalent cover the embodiments where this term is replaced with “consisting of” or “consisting essentially of”.

Within the present description, the expression “genomic position” intends to refer the unique location within the genome of a DNA feature, such as a given sequence or alteration. A genomic position defines the chromosome on which the DNA feature is located, starting and ending nucleotides positions, the length, and any other information which may be used to locate precisely and specifically a DNA feature within a genome. Genomic position encompasses “genomic coordinates”. Genomic coordinates or genomic interval consists of chromosome name and integers that together define a location (position or series of nucleotides) within a reference genome. The information specifically typically includes chromosome name, start position, end position and chromosome strand, optionally this information may include ‘+’ for the forward strand, ‘-’ for the reverse stranded, ‘.’ for both stranded/unstranded features. As example, genomic coordinates may be in the following format: chr1:1234570-1234870. For example, genomic coordinates of DNA elements may be obtained from the Encyclopedia of DNA elements (www.encodeproject.org; Kellis et al., Proc Natl Acad Sci USA. 2014; 111(17):6131-6138). In embodiments of the present disclosure, genomic position may be replaced with genomic coordinates.

Within the present description, the expression “genomic DNA” or gDNA intends to refer to the DNA which is found in the organisms' genome, usually within nucleus of cells, and which is passed on to offspring as information necessary for survival. It is distinguished from extrachromosomal DNAs, such as plasmids or mitochondrial DNA. Within the disclosure, a genomic DNA may be a tumor genomic DNA or non-tumor genomic DNA. A tumor genomic DNA is a DNA representative from the genomic DNA obtainable from tumor or cancer cells or tumor tissue sample. It may be obtained either from tumor or cancer cells taken from a tumor of a patient, or from blood circulating tumor DNA or from exosome. A non-tumor DNA is a DNA representative of non-tumor or non-cancer cells. It may be obtained from healthy constitutive cells or healthy constitutive tissue sample taken from a patient or a healthy individual.

Within the present description, the term “isolated” when used with respect to “cells”, “cancer cells”, “genomic DNA” or a “DNA element” intends to mean that the considered element, cell, cancer cell, genomic DNA, or else, is separated from its environment where it naturally occurs. The separation or isolation is usually carried out by means of technical methods or operations.

Within the present description, the term “p-value” intends to mean the probability of obtaining test results at least as extreme as the results actually observed, under the assumption that the null hypothesis is correct.

As used herein, the terms “patient”, “subject” or “individual” are used interchangeably and intend to refer to a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In one exemplary embodiment, a patient is a human.

As used herein, the terms “prevent” or “delay progression of” (and grammatical variants thereof) with respect to a disease or disorder relate to prophylactic treatment of a disease, e.g., in an individual suspected to have the disease, or at risk for developing the disease. A concerned disease or disorder is a cancer. As exemplary of concerned cancers one may cite sarcomas, such as leiomyosarcoma. Prevention may include, but is not limited to, preventing or delaying onset or progression of the disease and/or maintaining one or more symptoms of the disease at a desired or sub-pathological level.

Within the present description, the term “random model” intends to refer to a statistical model which models breakpoints distribution over the genome following a statistical distribution, especially a binomial distribution, wherein the random variable corresponds to the number of breakpoints over a given genomic sequence interval, and the model parameters are (n, p) in the case of a binomial distribution, with n the genomic sequence interval length or the DNA element length or the set of DNA elements length, and p the uniform probability equal to N/l, with N the total number of breakpoints and 1 the length of genomic sequence of reference.

Within the present description, the term “reference level” intends to refer to a level of said at least one predefined marker of DNA structure alteration, defined by the chosen random model.

Within the present description, the expression “reference threshold” intends to mean a value splitting a cohort of cancer patients in two groups having different properties and features representative of an evolution of said cancer.

Within the present description, the term “test score” intends to refer to a measure of the propensity of a given DNA element to break more than expected under random model, being especially a breakpoint hotspotness magnitude scale evaluating the propensity of such a DNA element to break more than expected under random breakage model.

Within the present description, the term “Replication-Associated Chromosomal INstability element” (RACINe) and “Transcription-Associated Chromosomal instability element” (TRACe) intends to refer to DNA elements for which the enrichment in marker of DNA structure alteration is either dependent (transcription-associated) of the presence of the DNA element in a gene or independent (replication-associated) of the presence of the DNA in a gene. A DNA element is considered present in a gene if it is present in the interval defined by the transcription start site (TSS) and its transcription end site (TES) of the gene or if it is overlapping with at least 1 bp of the interval defined by the TES and TSS of the gene.

As used herein, in the context of the present disclosure, the terms “treat”, “treatment” and the like refer to relief from or alleviation of pathological processes associated with a cancer. In the context of the present disclosure, insofar as it relates to any of the other conditions recited herein, the terms “treat”, “treatment”, and the like refer to relieving or alleviating one or more symptoms associated with such condition.

The list of sources, ingredients, and components as described hereinafter are listed such that combinations and mixtures thereof are also contemplated and within the scope herein.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

All lists of items, such as, for example, lists of ingredients, are intended to and should be interpreted as Markush groups. Thus, all lists can be read and interpreted as items “selected from the group consisting of” . . . list of items . . . “and combinations and mixtures thereof.”

Referenced herein may be trade names for components including various ingredients utilized in the present disclosure. The inventors herein do not intend to be limited by materials under any particular trade name. Equivalent materials (e.g., those obtained from a different source under a different name or reference number) to those referenced by trade name may be substituted and utilized in the descriptions herein.

### Methods for Classifying Cancer Patients

In one embodiment, the present disclosure relates to a method for classifying a patient having a cancer in a group representative of an evolution of said cancer, especially representative of a risk of metastasis of said cancer, especially for leiomyosarcoma, the method using:
i) a tumor genomic DNA sequence obtained by sequencing a tumor genomic DNA obtained from said patient,
ii) at least one set of preselected DNA elements, each DNA element having a genomic position, said set of DNA elements having a length L in base pairs,
iii) a preselected genomic DNA sequence of reference having a length l in base pairs, and
iv) at least one reference threshold separating two groups of patients, each group being representative of a different evolution of said cancer,
   said method comprising at least the steps of:
a) identifying, determining genomic positions, and quantifying a total quantity n of a predefined marker of DNA structure alteration within said tumor genomic DNA sequence of the tumor genomic DNA from said patient, by comparing said tumor genomic DNA sequence with said preselected genomic DNA sequence of reference,
b) associating, to the set of preselected DNA elements, a quantity of the predefined marker of DNA structure alteration, from the total quantity n, according to the genomic positions of the DNA elements of the set of preselected DNA elements and to the genomic positions of said predefined markers of DNA structure alteration obtained at step a),
c) computing a test score relative to a quantity of predefined markers of DNA structure alteration associated with the length L of said set of preselected DNA elements, said test score being based on a comparison between said quantity of predefined markers of DNA structure alteration and a reference level defined by a random model relative to the total quantity n of the predefined marker of DNA structure alteration over the length l of the preselected genomic DNA sequence of reference, and
d) comparing said test score to said at least one reference threshold in order to classify said patient.

### Genomic DNA

A method as disclosed herein comprises a use of a tumor genomic DNA sequence obtained by sequencing a tumor genomic DNA obtained from a patient and of a preselected genomic DNA sequence of reference having a length l in base pairs. A non-tumor genomic DNA sequence obtained by sequencing a non-tumor genomic DNA obtained from the same patient (or another healthy individual) may be used.

A method as disclosed herein may comprise a step of sequencing a tumor genomic DNA previously obtained from a cancer patient, to obtain a tumor genomic DNA sequence. Also, a method may comprise a step of sequencing a normal (or healthy or constitutive) genomic DNA previously obtained either from the patient from which the tumor genomic DNA is obtained (from healthy or normal or constitutive tissues or cells) or from a healthy patient.

The “genomic DNA”, or gDNA, is the DNA which is found in the organisms' genome and is passed on to offspring as information necessary for survival. It is distinguished from extrachromosomal DNAs, such as plasmids. A genomic DNA may be a tumor genomic DNA or a non-tumor genomic DNA.

A tumor genomic DNA is a genomic DNA isolated from tumor or cancer cells or from tumor or cancer tissue sample. A tumor genomic DNA may be isolated from tumor or cancer cells obtained from a cancer patient. The tumor cells or tissue taken from the patient may be processed to isolate the genomic DNA. Alternatively, the tumor cells obtained from the patient may be passaged in cell culture, at least once, and the passaged cells may then be processed to isolate the tumor genomic DNA.

A tumor genomic DNA may be the full-length sequence of a genomic DNA or a part of it. A part of genomic DNA may be any part, or a specific part identified by the presence of specific genes or DNA structures. For example, a part of a genomic DNA which may be used within the disclosure may be the exome, or a part of the exome, a group of genes, a chromosome, a group of chromosomes, or a part of a chromosome, or parts of different chromosomes. As used herein, the term “exome” intends to refer to the part of the genome consisting of exons that code information for protein synthesis. A part of the exome is composed of selected exons within the exome. Any part of the exome may be used within the disclosure.

Cancer cells may be isolated from patient's cancer or tumor by any known techniques in the art, for example using standard phenol-chloroform extraction protocol (Chomczynski et Sacchi 1987). For example, a sample of tumor or cancer tissue may be taken either by biopsy or from a patient's tissue removed after a surgery treatment. Cancer cells may be then isolated from surrounding tissue by enzymatic treatment using, for example, collagenase, hyaluronidase, trypsin, or mixture thereof. The enzymatic treatment may be followed by techniques of filtration and/or centrifugation to isolate the cancer cells. Cancer cells may be then used for genomic DNA extraction. Before genomic DNA extraction, isolated cancer cells may be amplified with a culture step and the obtained clonogenic colonies may be then isolated and used for genomic DNA extraction.

Before genomic DNA extraction, identity of the isolated cancer cells may be further determined or confirmed using known techniques such as cytometry and fluorescent tagged-antibodies specific to the cancer cell antigens.

Isolated cancer cells or isolated cancer tissue may be stored, for example as frozen cells or paraffin-embedded tissues, before use.

Alternatively again, a tumor genomic DNA may be obtained from circulating tumor DNA or circulating tumor cells which may be isolated from a blood sample taken from a cancer patient. Circulating tumor DNA (ctDNA) is found in the bloodstream and refers to DNA that comes from cancerous cells and tumors. Circulating tumor DNA may be isolated as disclosed in Board et al. (Ann NY Acad Sci. 2008 August; 1137:98-107). Circulating tumor cells may be isolated as disclosed in Sharma et al., Biotechnol Adv. 2018; 36(4):1063-1078).

Also, a tumor genomic DNA may be obtained from exosomes. Exosomes may be obtained as disclosed in Bai et al. (Nano-Micro Lett. 11, 59 (2019).)

In one embodiment, a method as disclosed herein may further comprise the use of a non-tumor genomic DNA sequence obtained by sequencing a genomic DNA obtained from non-cancer cells. The non-cancer cells may be obtained of a same patient from whom is obtained the tumor genomic DNA. Therefore, the non-tumor genomic DNA may be used as internal reference for further comparison. Alternatively, a non-tumor genomic DNA may be isolated from a healthy individual.

A non-tumor genomic DNA may be isolated from non-cancer or non-tumor cells or tissue samples. A non-tumor cell or tissue sample is identified as such as being presumably devoid of histological, cytological, or cytogenetic markers of cancer. Cancer and non-cancer tissues may be distinguished as disclosed in the reference books for the histological and molecular classification of tumors provided in the WHO Classification of Tumors series (publications.iarc.fr/Book-And-Report-Series/Who-Classification-Of-Tumors). As example of reference useable within the present disclosure one may cite the Soft Tissue and Bone Tumors—WHO Classification of Tumors, 5th Edition, Volume 3 (publications.iarc.fr/588).

When a part of the tumor genomic DNA is used, e.g., gene, group of genes, chromosome, group of chromosomes, part of chromosome, group of parts of chromosomes, exome, part of the exome, then the corresponding part in the preselected genomic DNA sequence is used for reference, e.g., gene, group of genes, chromosome, group of chromosomes, part of chromosome, group of parts of chromosomes, exome, part of the exome. For example, when the exome of a tumor genomic DNA is used, then the exome of a preselected genomic DNA may be used as reference. Alternatively, an exome of reference may be built using the UCSC tracks including GENECODE, and/or RefSeq, and/or LincRNATUCP and/or AUGUSTUS. One may use the exome, i.e., the totality of the exons, or a part of the exons. The exons may be combined or not with the untranslated regions, and/or may be combined or not with some base pairs around each exon, for example from 1 to 100, or from 1 to 50 base pairs around each exon.

It is possible to sequence all the genomic DNA and then to restrict the analysis on the part of interest. Alternatively, it may be possible to sequence only the part of interest with DNA targeted sequencing methods.

For further sequencing, a genomic DNA may be extracted from cells using any known techniques in the art. Genomic DNA extraction may comprise the steps of disrupting cytoplasmic and nuclear membranes, separating and purifying genomic DNA from other components of the cell lysate, such as lipids, proteins and other nucleic acids, and concentrating and purifying of the DNA. Various methods may be used as disclosed in Preetha et al., (2020-8(1). AJBSR) or in Schiebelhut et al. (Mol Ecol Resour. 2017 July; 17(4):721-729). For example, genomic DNA may be extracted using standard phenol-chloroform extraction protocol, for example as described in Chomczynski et Sacchi, Analytical Biochemistry 162 (1): 156-59, 1987.

Once extracted and isolated, an isolated genomic DNA may be sequenced to obtain a sequence or sequences of genomic DNA. The genomic DNA may be sequenced in whole or in part. Accordingly, in some embodiments, sequencing of an isolated genomic DNA may be carried out by a method selected among whole genomic DNA sequencing methods or DNA targeted sequencing methods.

The part of the genome to be sequenced and/or to be analyzed may be adapted upon factors such as the type of the cancer.

When sequenced in part, predefined or random regions of the genomic DNA are sequenced. Those regions may be or may comprise DNA elements as disclosed herein. The predefined regions may be contiguous or separated regions. A predefined region may comprise at least one DNA element. A predefined region may extend from 100 nucleotides, or below, for example down to 50 nucleotides, to about a million or more nucleotides. As example of targeted sequencing methods, one may mention exome-seq (ChIP-seq, RNA-seq). Exome-seq consists of polymerase chain reaction (PCR) amplified protein-coding regions of the genomic DNA.

In some embodiments, a tumor genomic DNA sequence may be an exome or a part of an exome.

A suitable exome or part of the exome to be used as a preselected genomic DNA sequence may be an exome or a part exome built with data from UCSC table browser using GENECODE, RefSeq, LincRNATUCP, AUGUSTUS, for example as indicated in the Examples section. One may use the exome, i.e., the totality of the exons, or a part of the exons, with full or portions of exons. The exons or part of the exons may be combined or not with the untranslated regions, and/or may be combined or not with some base pairs around each exon, for example from 1 to 100, or from 1 to 50 base pairs around each exon.

The sequencing of a genomic DNA in whole may be carried by any known techniques in the art. As examples of whole genome DNA sequencing techniques useful according to the present disclosure one may mention the Sanger methods, the Maxam & Gilbert methods, or next-generation sequencing (or high-throughput sequencing) methods, such as the pyrosequencing method or the method of single-molecule sequencing (SMS) as described in França, et al. (Q Rev Biophys. 2002 May; 35(2):169-200) or in Heather et al., (Genomics. 2016 January; 107(1):1-8), or sequencing-by-synthesis (SBS) chemistry as described in Minoche et al. (Genome Biol. 2011; 12(11):R112), or DNA optical mapping as described in Marie et al. (Proc Natl Acad Sci USA. 2018 Oct. 30; 115(44):11192-11197) or in Yuan et al. (Comput Struct Biotechnol J. 2020 Aug. 1; 18:2051-2062).

In one exemplary method, a sequence or sequences of an isolated genomic DNA to be used in the present disclosure may be obtained by sequencing-by-synthesis (SBS) chemistry, using for example HiSeq2000 and Genome Analyzer systems from Illumina. Such a method is more detailed in the Examples section.

To implement such exemplary method, short-insert paired-end libraries may be constructed. To obtain such libraries, an isolated genomic DNA may be sheared, size selected and concentrated in order to obtain DNA fragments of predefined size, for example of about 220 to about 480 bp. Fragmented DNA may be end-repaired, adenylated and ligated to specific indexed paired-end adapters. Then, DNA sequencing may be performed in paired-end mode, for example using HiSeq2000 or NovaSeq 6000 from Illumina. Images analysis, base calling and quality scoring of the run may be then processed, for example using the software Real Time Analysis (RTA 1.13.48), and followed by generation of FASTQ sequence files, using for example CASAVA (Illumina Inc., San Diego, CA, USA). DNA reads may be trimmed of the 5′ and 3′ low quality bases and sequencing adapters may be removed.

The then obtained sequence genomic DNA may be aligned and compared with preselected genomic DNA sequence of reference to identify, determine genomic positions, and quantification of a total number quantity n of a predefined marker of DNA structure alteration.

Within the present disclosure a genomic DNA of reference or a reference genome intends to refer to a digital nucleic acid sequence database representative of the set of genes in one idealized individual organism of a species. As it is assembled from the sequencing of DNA from a number of individual donors, a reference genome or genomic DNA of reference, including exomes of reference, does not represent the set of genes of any single individual organism. Instead, a reference provides a haploid mosaic of different DNA sequences from each donor. There are reference genomes for multiple species of viruses, bacteria, fungus, plants, and animals. A preselected genomic DNA of reference useable for the present disclosure may be for example the Human Genome version hg38 (or GRCh38) (genome.ucsc.edu or www.ncbi.nlm.nih.gov/grc/human or Schneider et al., Genome Research 27 (5): 849-64, 2017). The human reference genome, GRCh38, from the Genome Reference Consortium is derived from thirteen anonymous volunteers. Other genomic DNA of reference may be used, such as for example the Human Genome version hg19, or any other versions.

Reference genomes can be accessed online at several locations, using dedicated browsers such as Ensembl or UCSC Genome Browser. The length of a genome can be measured in multiple different ways. A simple way to measure genome length is to count the number of base pairs in the assembly.

A preselected genomic DNA sequence of reference may have a length l in base pairs (bp). In one embodiment, a preselected genomic DNA sequence of reference being the Human Genome version hg38 may have 2,948,611,470 bp.

For example, the DNA curated sequences obtained as above explained may be aligned, using for example bwa v-0.7.15 (H. Li et Durbin, Bioinformatics 25 (14): 1754-60, 2009), with default parameters, on the Human Genome version hg38 (genome.ucsc.edu or www.ncbi.nlm.nih.gov/grc/human or Schneider et al., Genome Research 27 (5): 849-64, 2017). Thus, aligned reads may be filtered out if their alignment score was less than a predefined threshold, for example 20, or if they are duplicated PCR reads, with for example SAMtools v1.3.1 (Heng Li et al., Bioinformatics (Oxford, England) 25 (16): 2078-79, 2009) and PicardTools v2.18.2 (Picard Toolkit.” 2019. Broad Institute, GitHub Repository. broadinstitute.github.io/picard/; Broad Institute), respectively.

### Predefined Markers of DNA Structure Alterations

A method as disclosed herein comprises a step of identifying, determining genomic positions, and quantifying a total quantity n of a predefined marker of DNA structure alteration within a tumor genomic DNA sequence of the tumor genomic DNA from a patient, by comparing said tumor genomic DNA sequence with a preselected genomic DNA sequence of reference.

In one embodiment, a method as disclosed herein may comprise aligning and comparing the tumor genomic DNA sequence, the non-tumor genomic DNA, and the preselected genomic DNA sequence of reference.

A predefined marker of DNA alteration is quantified per base pair of the length L of the set of the preselected DNA elements.

In one embodiment, a predefined marker of DNA structure alteration may be selected in the group comprising breakpoint, insertion, deletion, mutation, duplication, inversion, translocation, complex genomic rearrangements, copy number variation, telomeric allelic imbalance, large-scale state transitions, and loss of heterozygosity.

In one exemplary embodiment, a predefined marker of DNA structure alteration may be a breakpoint or a copy number variation.

A breakpoint may be either at a single chromosomal position (position of 1 nucleotide) corresponding to damage to DNA affecting a single chromosomal position, and may comprise single insertion or single deletion or single mutation, or at any of the genomic positions corresponding to at least, two chromosomal positions describing structural alteration affecting genomic intervals of at least 2 bp, any chromosome damages, such as insertion, deletion, duplication, mutation, inversion, translocation, complex genomic rearrangements copy number variation, telomeric allelic imbalance, large-scale state transitions, and loss of heterozygosity.

In one exemplary embodiment, a predefined marker of DNA structure alteration may be a breakpoint.

DNA structure alteration may be detected from paired tumor genomic DNA sequence/genomic DNA sequence of reference, possibly with non-tumor genomic DNA sequence. Alignment of DNA sequences, and identification and counting of DNA structure alterations may be carried out with any known techniques, such as alignment software. Numerous alignment software exist which can be used according to the present disclosure. For example, one may use database search software, pairwise alignment software, multiple sequence alignment software, genomic analysis software, or short-read sequence alignment software.

As for example of usable software, one may mention a Bowtie v2.2.1.0 (Langmead et al., Genome Biology 10 (3): R25, 2009), allowing soft-clipped sequences, or bwa v-0.0.15 (Li et Durbin, Bioinformatics 25 (14): 1754-60, 2009). The processing may comprise the steps:
i) identification of potential marker of DNA structure alteration, such as breakpoints,
ii) characterization of the second side of marker of DNA structure alteration, such as the breakpoints, and
iii) selection of high-confidence marker of DNA structure alteration.

As exemplary embodiment, when the marker of DNA structure alteration is a breakpoint, the processing steps may be as follows. At the identification step, reads with at least one soft-clipped end may be analyzed as singletons. A position may be considered as a potential breakpoint if it is covered by at least 4 soft-clipped reads, 5 soft-clipped bases (with at least two occurrences of two different bases), and if they represented more than 5% of the total amount of reads at this position in the tumor genomic DNA sequence. Potential somatic events may be selected by discarding positions covered by at least 1 read and 1 base in a surrounding 5-nucleotide window in the normal sample. They are referred to as the “first side” of the breakpoint.

At the characterization step, the genomic positions of the soft-clipped sequence from selected reads is determined by comparison with a genomic DNA sequence of reference, for example by using the UCSC blat server was used (Kent, Genome Research 12 (4): 656-64, 2002). If no match is returned, the reverse complement sequence may be pulled to test. If there is still no match, the BAM file may be investigated for some soft-clip somatic position around the discordant or oversized-insert read mate location from the first side of the breakpoint. Because of the small size of the soft-clipped sequence, multiple matches can be found. Soft-clipped abnormal read mates may be used to select matches with the most coherent chromosomic locations. They are referred to as the “second side” of the breakpoint.

At the selection step, positions detected from both the first and second sides (for example in a 5-nucleotide window) may be defined as a common pool. Couples of positions covered with reads and associated soft-clipped sequences separated by fewer than 15 nucleotides may be considered as artifacts (due to repeat regions for instance) and may be discarded. The breakpoints may be classified in three groups: high-confidence breakpoints, breakpoints needing investigation, and unique position breakpoints. If a breakpoint is covered by reads and associated soft-clipped sequences having both positions belonging to the common pool, it may be classified in the first group. If a breakpoint is covered by reads and associated soft-clipped sequences having only one of the positions belonging to the common pool, it may be classified in the second group. Then the missing position may be searched among the filtered positions. If it is present in the normal sample, the position is discarded, and the breakpoint is completed otherwise. Finally, the third group corresponding to breakpoints with both sides outside the common pool and considered as unique may be discarded.

The sides of breakpoints are sorted according to their chromosomic positions to avoid duplicates.

### DNA Elements of Reference and Biomarkers

A method as disclosed herein comprises a step of associating, to the set of preselected DNA elements, a quantity N of the predefined marker of DNA structure alteration, from the total quantity n, according to the genomic positions of the DNA elements of the set of preselected DNA elements and to the genomic positions of said predefined markers of DNA structure alteration obtained at step a).

As disclosed herein, in one embodiment, a set of preselected DNA elements may contain no-overlapping DNA elements.

A DNA element suitable for the disclosure may be any DNA element chosen on any structural, functional or sequence definition.

Herein, a DNA element intends to refer to a discrete genome segment that encodes a defined product (e.g., protein or non-coding RNA) or displays a reproducible biochemical signature (e.g., protein-binding, RNA-binding, chemical binding, or a specific chromatin or DNA structure). Data on DNA elements from human genome may be obtained from the Encyclopedia of DNA elements (www.encodeproject.org/; Kellis et al., Proc Natl Acad Sci USA. 2014; 111(17):6131-6138).

In one embodiment, DNA elements which may be used within the present disclosure may be selected among DNA repeats elements, Non-B DNA elements, regulatory DNA elements, or any structural or functional DNA or chromatin structure, and mixtures thereof.

DNA repeats (also known as repeated sequences, repetitive elements, repeating units, or repeats) are patterns of nucleic acids that occur in multiple copies throughout the genome. DNA repeats may comprise microsatellite (MS), Simple Repeats (SR), Low Complexity (LC), Self Chains segments (SCS), Long Terminal Repeats (LTR), and Retro Transposons (RT).

DNA microsatellite (MS) is a tract of repetitive DNA in which certain DNA motifs (ranging in length from one to six or more base pairs) are repeated, typically 5-50 times. Microsatellites may occur at thousands of locations within an organism's genome.

DNA Simple Repeats (SR) are DNA tracts in which a short base-pair motif is repeated several to many times in tandem (e.g. CAGCAGCAG).

DNA Low Complexity (LC) are often defined as regions of biased composition containing simple sequence repeats. Low-complexity sequences may be simple repeats such as ATATATATAT or regions that are highly enriched for just one letter, e.g. AAACAAAAAAAAGAAAAAAC.

DNA Self Chains segments (SCS) which were classified into self-chains segments self-aligned (SCS-S) and self-chains segments gaped (SCS-G). SCS is a group of short low copy repeats, for example less than 1 kb), which are chained self-aligned DNA sequences obtained by aligning the genome to itself. SCS-G correspond to the DNA segment encompassing any paired SCS in the same chromosome and their spacing gap. SCS-S correspond to self-aligned inverted self-chains.

DNA Long Terminal Repeats (LTR) are identical sequences of DNA that repeat hundreds or thousands of times found at either end of retrotransposons or proviral DNA formed by reverse transcription of retroviral RNA.

DNA Retro Transposons (RT) are transposons that are amplified via reverse transcription, i.e. the DNA elements are first transcribed into RNA and reverse-transcribed into DNA, and then inserted elsewhere in the genome

Non-B DNA may comprise A-Phased Repeats (APR), Direct Repeats (DR), G-quadruplex (GQ), Inverted Repeats (IR), Mirror Repeats (MR), Short Tandem Repeats (STR), Z-DNA (Z) and R-Loops Forming Sequences (RLFS).

A-Phased Repeats (APR) are defined as three or more tracts of four to nine adenines or adenines followed by thymines, with centers separated by 11-12 nucleotides.

DNA Direct Repeats (DR) are a type of genetic sequence that consists of two or more repeats of a specific sequence. In other words, the direct repeats are nucleotide sequences present in multiple copies in the genome.

DNA G-quadruplex (GQ) are higher-order DNA and RNA structures formed from G-rich sequences that are built around tetrads of hydrogen-bonded guanine bases.

DNA Inverted Repeats (IR) are single stranded sequences of nucleotides followed downstream by their reverse complements. These repeated DNA sequences often range from a pair of nucleotides to a whole gene, while the proximity of the repeat sequences varies between widely dispersed and simple tandem arrays

DNA Mirror Repeats (MR) are formed when the inverted sequence occurs within each individual strand of the DNA. Mirror repeats do not have complementary sequences within the same strand and cannot form hairpin or cruciform structures

DNA Short Tandem Repeats (STR) are accordion-like stretches of DNA containing core repeat units of between two and seven nucleotides in length that are tandemly repeated from approximately a half dozen to several dozen times.

Z-DNA (Z) are DNA in which the double helix has a left-handed rather than the usual right-handed twist and the sugar phosphate backbone follows a zigzag course.

DNA R-Loops Forming Sequences (RLFS) are DNA sequences able to form a three-stranded nucleic acid structure, composed of a DNA:RNA hybrid and the associated non-template single-stranded DNA.

Segmental duplications are long DNA sequences (typically defined as being >1 kb in length) that have nearly identical sequences (90-100%) and exist in multiple locations as a result of duplication events. SDs can be tandem or interspersed, and can be interchromosomal or intrachromosomal.

Interrupted repeats or nested repeats are DNA sequences repeats which have been interrupted by insertions of younger repeats or through local rearrangements.

interspersed repeats or interspersed repetitive DNA are found in all eukaryotic genomes. They differ from tandem repeat DNA in that rather than the repeat sequences coming right after one another, they are dispersed throughout the genome and nonadjacent. The sequence that repeats can vary depending on the type of organism, and many other factors.

A tandem repeat is a sequence of two or more DNA base pairs that is repeated in such a way that the repeats lie adjacent to each other on the chromosome. Tandem repeats are generally associated with non-coding DNA.

Short interspersed nuclear elements (SINEs) are non-autonomous, non-coding transposable elements (TEs) that are about 100 to 700 base pairs in length. They are a class of retrotransposons, DNA elements that amplify themselves throughout eukaryotic genomes, often through RNA intermediates.

Long interspersed nuclear elements (LINEs) are an ancient feature of around 6 kbp long that make up 20% of the genome and contain all the necessary information for self-transposition.

Transposons are small segments of DNA that range in length from hundreds to thousands of DNA base pairs which can move around to different positions in the genome of a single cell.

Satellite DNA consists of very large arrays of tandemly repeating, non-coding DNA. Satellite DNA is the main component of functional centromeres, and form the main structural constituent of heterochromatin.

Minisatellite DNA is a form of polymorphic DNA, comprising a variable number of tandem repeats, with repeat units of up to about 100 nucleotides in length, but typically 15-20 bp. In humans, minisatellites form clusters up to about 5 kb in length and are highly polymorphic due to the variation in repeat number.

Methylated DNA or methylome is the set of DNA sequences harboring DNA methylation.

A histone modification is a covalent post-translational modification (PTM) to histone proteins which includes methylation, phosphorylation, acetylation, ubiquitylation, and sumoylation. The PTMs made to histones can impact gene expression by altering chromatin structure or recruiting histone modifiers. It is intended to refer to the DNA sequences covered by those histone modifications.

Euchromatin is a form of chromatin that is lightly packed—as opposed to heterochromatin, which is densely packed. The presence of euchromatin usually reflects that cells are transcriptionally active. It is intended to refer to the DNA sequences covered by euchromatin.

Heterochromatin is a form of chromatin that is densely packed—as opposed to euchromatin, which is lightly packed. It is intended to refer to the DNA sequences covered by heterochromatin.

An insulator is a type of cis-regulatory element known as a long-range regulatory element. Found in multicellular eukaryotes and working over distances from the promoter element of the target gene, an insulator is typically 300 bp to 2000 bp in length. Insulators contain clustered binding sites for sequence specific DNA-binding proteins and mediate intra- and inter-chromosomal interactions.

Replication origin is a particular sequence in a genome where the process of replication is initiated.

Okazaki fragments are pieces of DNA that are transient components of lagging strand DNA synthesis at the replication fork.

Chromosome bands are bands produced on chromosomes by differential staining techniques. Depending on the particular staining technique, the bands are alternating light and dark or fluorescent and non-fluorescent. It is intended to refer to the DNA sequences covered by chromosome bands.

Regulatory DNA elements are elements involved in the regulation of gene expression and rely on the biochemical interactions involving DNA, the cellular proteins that make up chromatin, and transcription factors. Promoters and enhancers are the primary genomic regulatory components of gene expression.

Regulatory DNA elements may comprise CpG islands (CpGi), cis-regulatory modules (CRM), DNase I hypersensitive site (DHS) of promoter type (DHS_prom), DHS of enhancer type (DHS_enh), DHS of dyadic type (both enhancer and promoter signatures) (DHS_dyadic), DHS of other types (DHS_rest), transcription factor binding sites, and mixtures thereof.

CpG islands (CpGi) are regions of the genome that contain a large number of CpG dinucleotide repeats. In mammalian genomes, CpG islands usually extend for 300-3000 base pairs. They are located within and close to sites of about 40% of mammalian gene promoters. It is estimated that in mammalian genomes about 80% of CpG dinucleotides are methylated.

DNA cis-regulatory modules (CRM) are DNA sequence elements that have transcriptional regulatory activity. CRMs have usually 100-1000 DNA base pairs in length and are sequences where a number of transcription factors can bind and regulate expression of nearby genes and regulate their transcription rates. They are labeled as cis because they are typically located on the same DNA strand as the genes they control as opposed to trans, which refers to effects on genes not located on the same strand or farther away, such as transcription factors.

DNase I hypersensitive site (DHS) are regions of chromatin that are sensitive to cleavage by the DNase I enzyme. In these specific regions of the genome, chromatin has lost its condensed structure, exposing the DNA and making it accessible.

DHS of promoter type (DHS_prom) are DHS DNA regions with chromatin marks signature of type promoter.

DNA DHS of enhancer type (DHS_enh) are DHS DNA regions with chromatin marks signature of type enhancer.

DNA DHS of dyadic type (both enhancer and promoter signatures) (DHS_dyadic) are DHS DNA regions with chromatin marks signature of both enhancers and promoter.

DNA DHS of other types (DHS_rest) are DHS DNA regions with chromatin marks signature neither of promoter type nor of enhancer type nor of dyadic type.

Transcription factor binding sites are DNA sequence elements that a transcription factor binds to, and which short conserved sequences located within each promoter along the strands of DNA. Transcription factor binding sites which can be used may be for any transcriptional factors. For example, one may use transcription factors that are accessible in various databases such as the JASPAR transcription factor database (https://jaspar.genereg.net or UCSC table browser. Track: JASPAR transcription factors).

The genomic position or genomic coordinates of the DNA elements to be used within a method as disclosed may be obtained from various DNA and genomic database. For example, genomic coordinates of DNA elements may be obtained from the Encyclopedia of DNA elements (www.encodeproject.org/; Kellis et al., Proc Natl Acad Sci USA. 2014; 111(17):6131-6138).

Files with genomic coordinates for CpG islands, microsatellites, simple repeats, low complexity, retro-transposons, long terminal repeats, self-chains and sequencing gaps may be obtained from the UCSC Genome Browser website (genome-euro.ucsc.edu/cgi-bin/hgGateway?redirect=manual&source=genome.ucsc.edu). All Non-B DNA but RLFS may be generated using non-B DNA research tool of non-B DNA database (Cer et al., Nucleic Acids Research 41 (D1): D94-100, 2012). RLFS data may be generated using QmRLFS-finder (Jenjaroenpun, P. et al, Nucleic Acids Research 43 (W1): W527-34, 2015). CRM data may be obtained from Remap2018 (Chèneby et al., Nucleic Acids Research 46 (D1): D267-75, 2018) and data may be downloaded from (http://pedagogix-tagc.univ-mrs.fr/remap/). DNase I-accessible regulatory regions (with −log 10(p) >=2) may be downloaded from roadmap epigenomics project at personal.broadinstitute.org/meuleman/reg2map/HoneyBadger2_release/.

Self chains segments (SCS) may be prepared as in the article Zhou et al., Human Molecular Genetics 22 (13): 2642-51, 2013, but with small variations. The segment of any paired SCS in the same chromosome and their spacing gap may be defined as self-chains segments gaped (SCS-G). The self-aligned inverted SCS may be defined as self-chains segments self-aligned (SCS-S). The paired SCS located in different chromosomes and those in the same chromosome but having long spacing intervals (SCS size 30 kb) may be filtered out. In addition, any SCS-S/SCS-G overlapping with the human genome gaps, centromeres, or SDS may be further filtered out. To accurately count predefined markers of DNA structure alterations, any overlapping SCS-G may be merged into one SCS-G and the same procedure may be repeated for SCS-S.

Files with genomic coordinates for transcription factor binding sites may be available from databases such as the JASPAR transcription factor database (https://jaspar.genereg.net or UCSC table browser. Track: JASPAR transcription factors).

The selection of a DNA element to be used in a set of DNA elements in a method as disclosed herein, may be adapted depending on factors such as the type of cancer monitored, the responsiveness of a given cancer to a given treatment monitored, or the evolution of a given cancer (e.g., metastasis free survival) monitored. The selection of DNA elements to be used in a set of DNA elements may be carried out as disclosed herein by selecting a tested DNA element having a discriminating test score compared to a corresponding DNA element of reference.

In one exemplary embodiment, DNA elements which may be used within the present disclosure may be selected among DNA microsatellite (MS), DNA Simple Repeats (SR), DNA Low Complexity (LC), DNA Self Chains segments self-aligned (SCS-S), DNA self-chains segments gaped (SCS-G), DNA Long Terminal Repeats (LTR), DNA Retro Transposons (RT), A-Phased Repeats (APR), DNA Direct Repeats (DR), DNA G-quadruplex (GQ), DNA Inverted Repeats (IR), DNA Mirror Repeats (MR), DNA Short Tandem Repeats (STR), Z-DNA (Z), DNA R-Loops Forming Sequences (RLFS), CpG islands (CpGi), DNA cis-regulatory modules (CRM), DNase I hypersensitive site (DHS), such as DHS of promoter type (DHS_prom), DNA DHS of enhancer type (DHS_enh), DNA DHS of dyadic type (both enhancer and promoter signatures) (DHS_dyadic), DNA DHS of other types (DHS_rest), transcription factor binding sites, segmental duplications, interrupted repeats, interspersed repeats, tandem repeats, short interspersed nuclear element (SINE), long interspersed nuclear element (LINE), transposons, satellite DNA, mini-satellite DNA, methylated DNA, histone modifications, euchromatin, heterochromatin, insulators, replication origins, Okazaki fragments, chromosome bands, and combinations thereof. A set comprising all or part (i.e., a subset) of those elements, or a measure of the quantity of predefined marker of DNA structure alteration in all or part of those elements, or a statistical representation of the measure and distribution of the quantity of predefined marker of DNA structure alteration in all or part of those elements may define a biomarker which may be used in methods as disclosed herein.

In one exemplary embodiment, DNA elements which may be used within the present disclosure may be selected among direct repeats (DR), short tandem repeats (STR), mirror repeats (MR), inverted repeats (IR), Z DNA, simple repeats (SR), microsatellites (MS), low complexities (LC), R-loops forming sequences (RLFS), G-quadruplexes (GQ), CpG islands (CpGi), cis-regulated modules (CRM), self-chains segments self-aligned (SCS-S), DNase I hypersensitivity site (DHS) promoters (DHS_prom), DNase I hypersensitivity site (DHS) enhancers (DHS_enh), DNase I hypersensitivity site (DHS) dyadic regulatory elements (DHS_dyadic), and DNase I hypersensitivity site (DHS) of type rest (DHS rest), transcription factor binding sites, and mixtures thereof.

In another exemplary embodiment, DNA elements which may be used within the present disclosure may be selected among direct repeats (DR), short tandem repeats (STR), mirror repeats (MR), inverted repeats (IR), Z DNA, simple repeats (SR), microsatellites (MS), low complexities (LC), R-loops forming sequences (RLFS), G-quadruplexes (GQ), CpG islands (CpGi), cis-regulated modules (CRM), self-chains segments self-aligned (SCS-S), DNase I hypersensitivity site (DHS) promoters (DHS_prom), DNase I hypersensitivity site (DHS) enhancers (DHS_enh), DNase I hypersensitivity site (DHS) dyadic regulatory elements (DHS_dyadic), and transcription factor binding sites, and mixtures thereof.

In another exemplary embodiment, DNA elements which may be used within the present disclosure may be selected among direct repeats (DR), short tandem repeats (STR), mirror repeats (MR), inverted repeats (IR), Z DNA, simple repeats (SR), microsatellites (MS), low complexities (LC), R-loops forming sequences (RLFS), G-quadruplexes (GQ), CpG islands (CpGi), self-chains segments self-aligned (SCS-S), transcription factor binding sites, and mixtures thereof.

In one embodiment, a set of preselected DNA element may contain one DNA element. In another embodiment, a set of preselected DNA element may contain a plurality of DNA elements of same nature, e.g. only direct repeats, or only low complexities, or only CpG islands.

In another embodiment, a set of preselected DNA element may contain a plurality of DNA element of different nature.

In one exemplary embodiment, for example for Leiomyosarcoma (LMS), a set of preselected DNA elements may be a set of replication-associated chromosomal instability elements. Replication-associated chromosomal instability elements may be, for example, direct repeats (DR), short tandem repeats (STR), mirror repeats (MR), inverted repeats (IR), Z DNA, simple repeats (SR), microsatellites (MS), or low complexities (LC).

Herein, a replication-associated chromosomal instability element is an element for which its enrichment in a predefined marker of DNA structure alteration, such as breakpoint, is independent of its position inside a gene.

In one embodiment, a replication-associated chromosomal instability element may be selected among Direct Repeats (DR), Short Tandem Repeats (STR), Mirror Repeats (MR), Inverted Repeats (IR), Z DNA, Simple Repeats (SR), MicroSatellite (MS), Low Complexity (LC), or mixtures thereof. In one exemplary embodiment, a set of preselected DNA elements may consist in a set of replication-associated chromosomal instability elements, which may consist in Direct Repeats (DR), Short Tandem Repeats (STR), Mirror Repeats (MR), Inverted Repeats (IR), Z DNA, Simple Repeats (SR), MicroSatellite (MS), and Low Complexity (LC), and mixtures thereof. Such set of replication-associated chromosomal instability elements may define a biomarker which may be used in methods as disclosed herein.

Such sets, or indexes, may be called RACINi (or iRACIN).

When such sets, or indexes, are used with exomes, it may be called RACINi-exome (or iRACINexome).

In one exemplary embodiment, for example for Leiomyosarcoma (LMS), a set of preselected DNA elements may be a set of transcription-associated chromosomal instability elements. Transcription-associated chromosomal instability elements may be selected, for example, among R-loops forming sequences (RLFS), G-quadruplexes (GQ), CpG islands (CpGi), cis-regulated modules (CRM), self-chains segments self-aligned (SCS-S), DNase I hypersensitivity site (DHS) promoters (DHS_prom), DNase I hypersensitivity site (DHS) enhancers (DHS_enh), DNase I hypersensitivity site (DHS) dyadic regulatory elements (DHS_dyadic), DNase I hypersensitivity site (DHS) of type rest (DHS_rest), transcription factor binding sites, and mixtures thereof.

In one exemplary embodiment, for example for Leiomyosarcoma (LMS), a set of preselected DNA elements may be a set of transcription-associated chromosomal instability elements. Transcription-associated chromosomal instability elements may be selected, for example, among R-loops forming sequences (RLFS), G-quadruplexes (GQ), CpG islands (CpGi), cis-regulated modules (CRM), self-chains segments self-aligned (SCS-S), DNase I hypersensitivity site (DHS) promoters (DHS_prom), DNase I hypersensitivity site (DHS) enhancers (DHS_enh), DNase I hypersensitivity site (DHS) dyadic regulatory elements (DHS_dyadic), transcription factor binding sites, and mixtures thereof.

In one exemplary embodiment, for example for Leiomyosarcoma (LMS), a set of preselected DNA elements may be a set of transcription-associated chromosomal instability elements. Transcription-associated chromosomal instability elements may be selected, for example, among R-loops forming sequences (RLFS), G-quadruplexes (GQ), CpG islands (CpGi), self-chains segments self-aligned (SCS-S), transcription factor binding sites, and mixtures thereof.

Herein, a transcription-associated chromosomal instability element is an element for which its enrichment in predefined marker of DNA structure alterations, such as BP, is dependent of its position inside a gene. A DNA element is considered inside a gene if it is located within a gene interval or which overlap with at least one base-pair (bp) with a gene interval delimited by its transcription start site (TSS) and its transcription end site (TES).

In one embodiment, a transcription-associated chromosomal instability element may be selected among R-Loops Forming Sequences (RLFS), G-quadruplex (GQ), CpG islands (CpGi), cis-regulatory modules (CRM), self-chains segments self-aligned (SCS-S), DNase I hypersensitivity site (DHS) promoters (DHS_prom), DNase I hypersensitivity site (DHS) enhancers (DHS_enh), and DNase I hypersensitivity site (DHS) dyadic regulatory elements (DHS_dyadic), DNase I hypersensitivity site (DHS) of type rest (DHS_rest), transcription factor binding sites, and mixtures thereof. In one exemplary embodiment, a set of preselected DNA elements may consist in a set of transcription-associated chromosomal instability elements, which may consist in R-Loops Forming Sequences (RLFS), G-quadruplex (GQ), CpG islands (CpGi), cis-regulatory modules (CRM), self-chains segments self-aligned (SCS-S), and DNase I hypersensitivity site (DHS) promoters (DHS_prom), DNase I hypersensitivity site (DHS) enhancers (DHS_enh), DNase I hypersensitivity site (DHS) dyadic regulatory elements (DHS_dyadic), and DNase I hypersensitivity site (DHS) of type rest (DHS_rest). In one exemplary embodiment, a set of preselected DNA elements may consist in a set of transcription-associated chromosomal instability elements, which may consist in R-Loops Forming Sequences (RLFS), G-quadruplex (GQ), CpG islands (CpGi), cis-regulatory modules (CRM), self-chains segments self-aligned (SCS-S), and DNase I hypersensitivity site (DHS) promoters (DHS_prom), DNase I hypersensitivity site (DHS) enhancers (DHS_enh), and DNase I hypersensitivity site (DHS) dyadic regulatory elements (DHS_dyadic).

In some embodiments, a set of preselected DNA elements may consist in a set of transcription-associated chromosomal instability elements, which may consist in R-Loops Forming Sequences (RLFS), G-quadruplex (GQ), CpG islands (CpGi), cis-regulatory modules (CRM), self-chains segments self-aligned (SCS-S), and DNase I hypersensitivity site (DHS) promoters (DHS_prom), DNase I hypersensitivity site (DHS) enhancers (DHS_enh), DNase I hypersensitivity site (DHS) dyadic regulatory elements (DHS_dyadic), DNase I hypersensitivity site (DHS) of type rest (DHS_rest), and JASPAR transcription factor binding sites. In some embodiments, a set of preselected DNA elements may consist in a set of transcription-associated chromosomal instability elements, which may consist in R-Loops Forming Sequences (RLFS), G-quadruplex (GQ), CpG islands (CpGi), cis-regulatory modules (CRM), self-chains segments self-aligned (SCS-S), and DNase I hypersensitivity site (DHS) promoters (DHS_prom), DNase I hypersensitivity site (DHS) enhancers (DHS_enh), DNase I hypersensitivity site (DHS) dyadic regulatory elements (DHS_dyadic), and transcription factor binding sites.

In one exemplary embodiment, a set of preselected DNA elements may consist in a set of transcription-associated chromosomal instability elements, which may consist in R-Loops Forming Sequences (RLFS), G-quadruplex (GQ), CpG islands (CpGi), self-chains segments self-aligned (SCS-S), and transcription factor binding sites.

Such sets of transcription-associated chromosomal instability elements may define a biomarker which may be used in methods as disclosed herein.

Such sets, or indexes, may be called TRACi (or iTRAC).

When such sets, or indexes, are used with exomes, it may be called TRACi-exome (or iTRACexome).

In one exemplary embodiment, a set of preselected DNA elements which may be used within the present disclosure may be or may consist in replication-associated chromosomal instability elements, in transcription-associated chromosomal instability elements, or in mixtures thereof.

In another exemplary embodiment, for example for Leiomyosarcoma (LMS), a set of preselected DNA elements which may be used within the present disclosure may be or may consist in transcription-associated chromosomal instability elements or in a mixture of replication-associated chromosomal instability elements and transcription-associated chromosomal instability elements.

In one exemplary embodiment, for example for Leiomyosarcoma (LMS), a set of preselected DNA elements which may be used within the present disclosure may be or may consist in a mixture of replication-associated chromosomal instability elements and transcription-associated chromosomal instability elements. Such mixture may consist in Direct Repeats (DR), Short Tandem Repeats (STR), Mirror Repeats (MR), Inverted Repeats (IR), Z DNA, Simple Repeats (SR), MicroSatellite (MS), and Low Complexity (LC), in R-Loops Forming Sequences (RLFS), G-quadruplex (GQ), CpG islands (CpGi), cis-regulatory modules (CRM), self-chains segments self-aligned (SCS-S), DNase I hypersensitivity site (DHS) promoters (DHS_prom), DNase I hypersensitivity site (DHS) enhancers (DHS_enh), DNase I hypersensitivity site (DHS) dyadic regulatory elements (DHS_dyadic), DNase I hypersensitivity site (DHS) of type rest (DHS_rest), and transcription factor binding sites. Such set of elements, or measure of the quantity of predefined marker of DNA structure alteration in this set of elements, or statistical representation of the measure and distribution of the quantity of predefined marker of DNA structure alteration in this element may define a biomarker which may be used in methods as disclosed herein.

In one exemplary embodiment, for example for Leiomyosarcoma (LMS), a set of preselected DNA elements which may be used within the present disclosure may be or may consist in a mixture of replication-associated chromosomal instability elements and transcription-associated chromosomal instability elements. Such mixture may consist in Direct Repeats (DR), Short Tandem Repeats (STR), Mirror Repeats (MR), Inverted Repeats (IR), Z DNA, Simple Repeats (SR), MicroSatellite (MS), and Low Complexity (LC), and in R-Loops Forming Sequences (RLFS), G-quadruplex (GQ), CpG islands (CpGi), cis-regulatory modules (CRM), self-chains segments self-aligned (SCS-S), DNase I hypersensitivity site (DHS) promoters (DHS_prom), DNase I hypersensitivity site (DHS) enhancers (DHS_enh), DNase I hypersensitivity site (DHS) dyadic regulatory elements (DHS_dyadic), and transcription factor binding sites. Such set of elements, or measure of the quantity of predefined marker of DNA structure alteration in this set of elements, or statistical representation of the measure and distribution of the quantity of predefined marker of DNA structure alteration in this elements may define a biomarker which may be used in methods as disclosed herein.

In one exemplary embodiment, for example for Leiomyosarcoma (LMS), a set of preselected DNA elements which may be used within the present disclosure may be or may consist in a mixture of replication-associated chromosomal instability elements and transcription-associated chromosomal instability elements. Such mixture may consist in Direct Repeats (DR), Short Tandem Repeats (STR), Mirror Repeats (MR), Inverted Repeats (IR), Z DNA, Simple Repeats (SR), MicroSatellite (MS), and Low Complexity (LC), and in R-Loops Forming Sequences (RLFS), G-quadruplex (GQ), CpG islands (CpGi), self-chains segments self-aligned (SCS-S), and transcription factor binding sites. Such set of elements, or measure of the quantity of predefined marker of DNA structure alteration in this set of elements, or statistical representation of the measure and distribution of the quantity of predefined marker of DNA structure alteration in this elements may define a biomarker which may be used in methods as disclosed herein.

A set of preselected DNA elements, or index, may be built by combining different DNA elements as indicated herein.

The set of preselected DNA elements may be applied on the whole genomic DNA sequence, or a part of it, as for example the exome, a part of the exome, a gene, a group of gene, a chromosome, a group of chromosomes, a part of chromosome, or a group of parts of chromosome or chromosomes.

The selected DNA elements used to build the set of preselected DNA elements, or index may have overlapping genomic coordinates or position, that is a selected DNA element may overlap in all or in part with another selected DNA element. Two DNA elements are considered as being overlapped if they have at least one base pair in common.

In one embodiment, predefined markers of DNA alterations are to be quantified once over the overlapping parts of DNA elements. Several means are available to quantify once the predefined marker of DNA alterations.

One means is to use sets of preselected DNA elements, or indexes, with non-overlapping DNA elements. Such sets or indexes may be built by using selected non-overlapping DNA elements. Alternatively, such sets or indexes may be built using selected overlapping and non-overlapping DNA elements, and by merging overlapping DNA elements. Such sets or indexes will contain merged and non-merged DNA elements, and no-overlapping DNA elements.

In one embodiment, the selected DNA elements with at least one overlapping base-pair may be merged.

For example, for each selected DNA element of the set or index a BED (Browser Extended Data) file is built. Then all BED files of the selected DNA elements may be pooled into one BED file, and each DNA element may sorted according to intervals positions (bedtools), and all overlapping intervals are merged (BEDtools) to obtain the preselected set of DNA elements or index (bedtools.readthedocs.io/en/latest/index.html).

Each of the obtained merged DNA element has a length le in base pairs and a genomic position or coordinates.

In one exemplary embodiment, the methods as disclosed herein comprise the use of a preselected set of DNA elements of reference with no-overlapping DNA elements (for example with merged and non-merged DNA elements) and their genomic position or coordinates.

Another mean to count once the predefined marker of DNA structure alterations in overlapping DNA elements of a set of preselected DNA elements is to quantify all the predefined markers of DNA alterations in all the DNA elements of the selected DNA elements. The overlapping parts of DNA elements may be identified with BEDtools as disclosed herein. Then, the quantity of predefined markers of DNA structure alterations associated with each overlapping part so identified is subtracted from the quantity of predefined markers of DNA structure alterations to obtain the total quantity n of predefined markers of DNA structure alterations.

In one embodiment a set of preselected DNA elements as disclosed herein may be a biomarker of cancer, for example a biomarker of cancer evolution. In one embodiment a biomarker may be a measure of the quantity of predefined marker of DNA structure alteration, such as breakpoints, in a set of preselected DNA elements, as disclosed herein. In some embodiments, a biomarker may be a statistical representation of the measure and distribution of the quantity of predefined marker of DNA structure alteration, such as breakpoints, in a set of preselected DNA elements.

In one exemplary embodiment, a biomarker as disclosed herein, e.g., a set of preselected DNA elements, may be a biomarker of an evolution of a cancer in a patient having said cancer.

In one exemplary embodiment, for example for Leiomyosarcoma (LMS), such a biomarker may consist in replication-associated chromosomal instability elements, and especially may be a set of elements consisting in direct repeats (DR), short tandem repeats (STR), mirror repeats (MR), inverted repeats (IR), Z DNA, simple repeats (SR), microsatellites (MS), and low complexities (LC). Such biomarker or set of preselected DNA elements may be named RACINi (or iRACIN). A biomarker may be a measure of the quantity of predefined marker of DNA structure alteration, such as breakpoints, in such set of preselected DNA elements. In some embodiments, a biomarker may be a statistical representation of the measure and distribution of the quantity of predefined marker of DNA structure alteration, such as breakpoints, in such set of preselected DNA elements.

In another exemplary embodiment, for example for Leiomyosarcoma (LMS), such a biomarker may consist in transcription-associated chromosomal instability elements, and especially may be a set of elements consisting in R-loops forming sequences (RLFS), G-quadruplexes (GQ), CpG islands (CpGi), cis-regulated modules (CRM), self-chains segments self-aligned (SCS-S), DNase I hypersensitivity site (DHS) promoters (DHS_prom), DNase I hypersensitivity site (DHS) enhancers (DHS_enh), DNase I hypersensitivity site (DHS) dyadic regulatory elements (DHS_dyadic), and DNase I hypersensitivity site (DHS) of type rest (DHS_rest). In another exemplary embodiment, such a biomarker may consist in transcription-associated chromosomal instability elements, and especially may be a set of elements consisting in R-loops forming sequences (RLFS), G-quadruplexes (GQ), CpG islands (CpGi), cis-regulated modules (CRM), self-chains segments self-aligned (SCS-S), DNase I hypersensitivity site (DHS) promoters (DHS_prom), DNase I hypersensitivity site (DHS) enhancers (DHS_enh), and DNase I hypersensitivity site (DHS) dyadic regulatory elements (DHS_dyadic). A biomarker may be a measure of the quantity of predefined marker of DNA structure alteration, such as breakpoints, in such set of preselected DNA elements. In some embodiments, a biomarker may be a statistical representation of the measure and distribution of the quantity of predefined marker of DNA structure alteration, such as breakpoints, in such set of preselected DNA elements.

Alternatively, in some embodiments, for example for Leiomyosarcoma (LMS), a biomarker consisting in transcription-associated chromosomal instability elements may be a set of elements consisting in R-loops forming sequences (RLFS), G-quadruplexes (GQ), CpG islands (CpGi), cis-regulated modules (CRM), self-chains segments self-aligned (SCS-S), DNase I hypersensitivity site (DHS) promoters (DHS_prom), DNase I hypersensitivity site (DHS) enhancers (DHS_enh), DNase I hypersensitivity site (DHS) dyadic regulatory elements (DHS_dyadic), DNase I hypersensitivity site (DHS) of type rest (DHS rest), and transcription factor binding sites. A biomarker may be a measure of the quantity of predefined marker of DNA structure alteration, such as breakpoints, in such set of preselected DNA elements. In some embodiments, a biomarker may be a statistical representation of the measure and distribution of the quantity of predefined marker of DNA structure alteration, such as breakpoints, in such set of preselected DNA elements.

In some embodiments, for example for Leiomyosarcoma (LMS), a biomarker consisting in transcription-associated chromosomal instability elements may be a set of elements consisting in R-loops forming sequences (RLFS), G-quadruplexes (GQ), CpG islands (CpGi), cis-regulated modules (CRM), self-chains segments self-aligned (SCS-S), DNase I hypersensitivity site (DHS) promoters (DHS_prom), DNase I hypersensitivity site (DHS) enhancers (DHS_enh), and DNase I hypersensitivity site (DHS) dyadic regulatory elements (DHS_dyadic), and transcription factor binding sites. A biomarker may be a measure of the quantity of predefined marker of DNA structure alteration, such as breakpoints, in such set of preselected DNA elements. In some embodiments, a biomarker may be a statistical representation of the measure and distribution of the quantity of predefined marker of DNA structure alteration, such as breakpoints, in such set of preselected DNA elements.

In some embodiments, for example for Leiomyosarcoma (LMS), a biomarker consisting in transcription-associated chromosomal instability elements may be a set of elements consisting in R-loops forming sequences (RLFS), G-quadruplexes (GQ), CpG islands (CpGi), self-chains segments self-aligned (SCS-S), and transcription factor binding sites. A biomarker may be a measure of the quantity of predefined marker of DNA structure alteration, such as breakpoints, in such set of preselected DNA elements. In some embodiments, a biomarker may be a statistical representation of the measure and distribution of the quantity of predefined marker of DNA structure alteration, such as breakpoints, in such set of preselected DNA elements.

Such biomarkers or sets of preselected DNA elements may be named TRACi (or iTRAC).

In another exemplary embodiment, for example for Leiomyosarcoma (LMS), such a biomarker may consist in a combination of transcription-associated chromosomal instability elements and replication-associated chromosomal instability elements, and especially may be a set of elements consisting in direct repeats (DR), short tandem repeats (STR), mirror repeats (MR), inverted repeats (IR), Z DNA, simple repeats (SR), microsatellites (MS), low complexities (LC), R-loops forming sequences (RLFS), G-quadruplexes (GQ), CpG islands (CpGi), cis-regulated modules (CRM), self-chains segments self-aligned (SCS-S), DNase I hypersensitivity site (DHS) promoters (DHS_prom), DNase I hypersensitivity site (DHS) enhancers (DHS_enh), DNase I hypersensitivity site (DHS) dyadic regulatory elements (DHS_dyadic), and DNase I hypersensitivity site (DHS) of type rest (DHS_rest). A biomarker may be a measure of the quantity of predefined marker of DNA structure alteration, such as breakpoints, in such set of preselected DNA elements. In some embodiments, a biomarker may be a statistical representation of the measure and distribution of the quantity of predefined marker of DNA structure alteration, such as breakpoints, in such set of preselected DNA elements

In another exemplary embodiment, for example for Leiomyosarcoma (LMS), such a biomarker may be a combination of transcription-associated chromosomal instability elements and replication-associated chromosomal instability elements, and especially may be a set of elements consisting in direct repeats (DR), short tandem repeats (STR), mirror repeats (MR), inverted repeats (IR), Z DNA, simple repeats (SR), microsatellites (MS), low complexities (LC), R-loops forming sequences (RLFS), G-quadruplexes (GQ), CpG islands (CpGi), cis-regulated modules (CRM), self-chains segments self-aligned (SCS-S), DNase I hypersensitivity site (DHS) promoters (DHS_prom), DNase I hypersensitivity site (DHS) enhancers (DHS_enh), and DNase I hypersensitivity site (DHS) dyadic regulatory elements (DHS_dyadic). A biomarker may be a measure of the quantity of predefined marker of DNA structure alteration, such as breakpoints, in such set of preselected DNA elements. In some embodiments, a biomarker may be a statistical representation of the measure and distribution of the quantity of predefined marker of DNA structure alteration, such as breakpoints, in such set of preselected DNA elements

In some embodiments, for example for Leiomyosarcoma (LMS), such a biomarker may consist in a combination of transcription-associated chromosomal instability elements and replication-associated chromosomal instability elements, and especially may be a set of elements consisting in direct repeats (DR), short tandem repeats (STR), mirror repeats (MR), inverted repeats (IR), Z DNA, simple repeats (SR), microsatellites (MS), low complexities (LC), R-loops forming sequences (RLFS), G-quadruplexes (GQ), CpG islands (CpGi), cis-regulated modules (CRM), self-chains segments self-aligned (SCS-S), DNase I hypersensitivity site (DHS) promoters (DHS_prom), DNase I hypersensitivity site (DHS) enhancers (DHS_enh), DNase I hypersensitivity site (DHS) dyadic regulatory elements (DHS_dyadic), DNase I hypersensitivity site (DHS) of type rest (DHS_rest), and transcription factor binding sites. A biomarker may be a measure of the quantity of predefined marker of DNA structure alteration, such as breakpoints, in such set of preselected DNA elements. In some embodiments, a biomarker may be a statistical representation of the measure and distribution of the quantity of predefined marker of DNA structure alteration, such as breakpoints, in such set of preselected DNA elements

In some embodiments, for example for Leiomyosarcoma (LMS), such a biomarker may be a combination of transcription-associated chromosomal instability elements and replication-associated chromosomal instability elements, and especially may be a set of elements consisting in direct repeats (DR), short tandem repeats (STR), mirror repeats (MR), inverted repeats (IR), Z DNA, simple repeats (SR), microsatellites (MS), low complexities (LC), R-loops forming sequences (RLFS), G-quadruplexes (GQ), CpG islands (CpGi), cis-regulated modules (CRM), self-chains segments self-aligned (SCS-S), DNase I hypersensitivity site (DHS) promoters (DHS_prom), DNase I hypersensitivity site (DHS) enhancers (DHS_enh), DNase I hypersensitivity site (DHS) dyadic regulatory elements (DHS_dyadic), and transcription factor binding sites. A biomarker may be a measure of the quantity of predefined marker of DNA structure alteration, such as breakpoints, in such set of preselected DNA elements. In some embodiments, a biomarker may be a statistical representation of the measure and distribution of the quantity of predefined marker of DNA structure alteration, such as breakpoints, in such set of preselected DNA elements

In some embodiments, for example for Leiomyosarcoma (LMS), such a biomarker may be a combination of transcription-associated chromosomal instability elements and replication-associated chromosomal instability elements, and especially may be a set of elements consisting in direct repeats (DR), short tandem repeats (STR), mirror repeats (MR), inverted repeats (IR), Z DNA, simple repeats (SR), microsatellites (MS), low complexities (LC), R-loops forming sequences (RLFS), G-quadruplexes (GQ), CpG islands (CpGi), self-chains segments self-aligned (SCS-S), and transcription factor binding sites. A biomarker may be a measure of the quantity of predefined marker of DNA structure alteration, such as breakpoints, in such set of preselected DNA elements. In some embodiments, a biomarker may be a statistical representation of the measure and distribution of the quantity of predefined marker of DNA structure alteration, such as breakpoints, in such set of preselected DNA elements

In some embodiments, for example for Leiomyosarcoma (LMS), a biomarker or set of preselected DNA elements may be a mixture of RACINi (or iRACIN) and TRACi (aka iTRAC).

A biomarker may be a measure of the quantity of predefined marker of DNA structure alteration, such as breakpoints, in a set of preselected DNA elements, as disclosed herein. In some embodiments, a biomarker may be a statistical representation of the measure and distribution of the quantity of predefined marker of DNA structure alteration, such as breakpoints, in a set of preselected DNA elements

A method as disclosed herein comprises a step of associating, to the set of preselected DNA elements, a quantity N, from the total quantity n, of the quantified predefined marker of DNA structure alterations.

By comparing the genomic position or coordinates of each DNA element of the set of DNA elements with the genomic positions or coordinates of the quantified predefined markers of DNA structure alteration it is possible to associate with each DNA elements a quantity (or a fraction) N₁, N₂, N₃, . . . , from the total quantity n of the predefined marker of DNA structure alteration.

In one embodiment, when the predefined marker of DNA structure alteration is a breakpoint, a quantity of the predefined marker of DNA structure alteration may be associated to each DNA element as a quantity N₁, N₂, N₃, . . . of the predefined marker of DNA structure alteration, from the total quantity n, said association being according to the genomic positions of said DNA element and to the genomic positions of the predefined markers of DNA structure alteration.

In one embodiment, a method as disclosed herein may comprising a step of summing the quantities N₁, N₂, N₃, . . . , from the total quantity n, to obtain a quantity N of predefined markers of DNA structure alteration associated with the set of preselected DNA elements. Such step may be carried out after a step of associating, to each element of the set of preselected DNA elements, a quantity N₁, N₂, N₃, . . . , of the predefined marker of DNA structure alteration and before a step of computing a test score.

In some embodiments, a biomarker may be a statistical representation, or a test score, of the quantity of markers of DNA structure alteration in set of preselected DNA elements, as above defined, in tumor genomic DNA sequence.

### Random Model, Test Scores and Thresholds

### Random Model and Test Scores

A method as disclosed herein comprises a step of computing a test score relative to a quantity of predefined markers of DNA structure alteration associated with the length L of said set of preselected DNA elements, the test score being based on a comparison between said quantity of predefined markers of DNA structure alteration and a reference level defined by a random model relative to the total quantity n of the predefined marker of DNA structure alteration over the length l of the preselected genomic DNA sequence of reference.

A method as disclosed herein further comprises a step of comparing the test score to at least one reference threshold in order to classify a patient.

The method according to the invention thus is a statistical approach, an “a posteriori” approach in which it is not necessary to search for and to use a specific gene or a specific DNA structure alteration. Neither using all the alterations that had occurred nor using only alterations which are known to have occurred without a doubt is needed, since the statistical aspect of the method according to the invention allows having statistical biomarkers less prone to give false negative or false positive results.

The distribution of the quantity n of the predefined marker of DNA structure alteration over the length l of the preselected genomic DNA sequence of reference may be used as a reference level to be compared with the distribution of the quantity N of predefined marker of DNA structure alteration over the length L of the set of preselected DNA elements. A difference observed between the two distributions may be used to compute a test score.

In one embodiment, said random model may use a binomial, Poisson, beta or normal, power law distributions, chi-square, or any statistical distribution.

In a preferred embodiment, said random model may be a random breakage model, for example using a binomial distribution. A random breakage model may be used to evaluate the propensity of a DNA element or a set of preselected DNA elements to break more than expected. Such model may be used with breakpoints as a predefined marker of DNA structure alteration.

A reference level may be a level of the predefined marker of DNA structure alteration as defined by the chosen random model.

The uniform probability p of any position within a genomic DNA sequence of length of reference l in base pairs to carry a predefined marker of DNA structure alteration, such as a breakpoint, may be computed as the total quantity n of predefined marker of DNA structure alterations, e.g. BP, (n) divided by l: p=n/l. p represents the uniform distribution probability of the total quantity n of the quantified predefined marker of DNA structure alteration over the length l of a genomic DNA sequence of reference

In one embodiment, the test score may use a probability p of having a quantity N of predefined markers of DNA structure alteration associated with the length L of the set of preselected DNA elements, the probability p being computed with the random model defining a reference level and using a probability pᵤ relative to an isotropic distribution of the total quantity n of the predefined marker of DNA structure alteration over the length l of the preselected genomic DNA sequence of reference.

In another embodiment, a test score may use a probability p of having a quantity N of predefined markers of DNA alteration within a length L of a set of preselected DNA elements, and a probability pᵤ which may be a ratio of the total quantity n of identified and quantified predefined markers of DNA structure alteration to the length l of the preselected genomic DNA sequence of reference.

In one embodiment, probability pᵤ may be relative to a uniform distribution.

In one embodiment, for a given set of preselected DNA elements of length (L) and a given quantity N of quantified predefined marker of DNA structure alteration associated with the set of preselected DNA elements, a probability of the set of preselected DNA elements to harbor more predefined marker of alterations than expected under a random breakage model (RBM) may be computed by the probability mass function of the binomial function:

$$f ( x ) = ( \begin{matrix}n \\ x\end{matrix} ) {{p}^{x} ( 1 - p )}^{( n - x )}$$

where x=N, n=L, p=pu.

The probability of observing more than N breakpoints (BP) under RBM is defined as p(x>N)=1−p(x<=N)=1−Σᵢ₌₀ⁿf(i), where n=N.

A test score, or Hscore in the Examples section, may be computed as being the −log 10 of the probability (p) computed as disclosed herein. This test score or Hscore translates the predefined marker hotspotness magnitude scale for the propensity of the considered set of preselected of DNA elements to be more altered, or to break more, than expected by chance under RBM.

In one embodiment, a probability p of having the quantity of predefined markers of DNA structure alteration associated with the length L of the set of preselected DNA elements is a one-sided p-value wherein only the values superior to the quantity of predefined markers of DNA structure alteration are taken into account, and the test score is computed as a logarithmic transformation of said p-value.

In variants, said test score may be a non-probability-based score, being especially a ratio or a difference. Said test score may be in the form of a numerical value, for example a value comprised between 0 and 10. In a variant, the score is in the form of a letter.

In embodiments of methods as disclosed herein, the obtained test score, or Hscore, is then compared to at least one reference threshold in order to classify the cancer patient in one group among at least said two groups representative of a different evolution of said cancer.

### Reference Thresholds

A reference threshold is determined relative to a set of preselected DNA elements. A reference threshold separate groups of patients according to the distribution of the quantity of predefined markers of DNA structure alteration in the set of preselected DNA elements. For example, a threshold reference may be defined with respect to a set of preselected DNA elements which may be either TRACe or RACINe, as disclosed herein. For example, a reference threshold may be a −log 10 of the probability of random distribution of the quantified predefined marker of DNA structure alteration in the set of preselected DNA elements, computed using the RBM as disclosed herein, and may separate groups of patients with different probabilities of DNA alterations in this set of DNA elements.

A reference threshold may separate two groups representative of a different evolution of said cancer.

A reference threshold may be relative to a plurality of distributions, in a set of preselected DNA elements, of at least one quantified predefined marker of DNA structure alteration, the quantification and genomic position or coordinates of which being previously obtained from a plurality of tumor genomic DNA sequences representative of said cancer. The quantification and genomic position or coordinates of the quantified predefined marker of DNA structure alteration may be obtained as disclosed herein.

Each distribution of the plurality of distribution, in a set of preselected DNA elements, of the predefined marker of DNA alterations is determined for each tumor genomic DNA sequence may be determined as disclosed herein. The plurality of distributions may be used to obtain a plurality of test scores or Hscores as described herein.

The reference threshold is then obtained by using a classifier trained on the plurality of distributions or test scores of the quantified predefined marker of DNA structure alteration obtained on a plurality of sequences obtained from several isolated genomic DNA. Clinical data associated to each patient of said plurality of patients having said cancer may be also used during the learning of the classifier. In a preferred embodiment, said clinical data may be information concerning the fact that said patient had a metastasis, and the date of such metastasis, and any treatments undergone by the patient. Said clinical data may also be the age and/or the gender of said patient.

### Methods for Classifying Cancer Patients and Determining Threshold(s) Representative of an Evolution of a Cancer

In one embodiment, a method as disclosed herein may be a method for classifying, or stratifying, a patient having a cancer in a group representative of an evolution of said cancer, especially representative of a risk of metastasis of said cancer, especially for leiomyosarcoma, and the method may use:
i) a tumor genomic DNA sequence obtained by sequencing a tumor genomic DNA obtained from said patient,
ii) at least one set of preselected DNA elements, each DNA element having a genomic position, said set of DNA elements having a length L in base pairs,
iii) a preselected genomic DNA sequence of reference having a length l in base pairs, and
iv) at least one reference threshold separating two groups of patients, each group being representative of a different evolution of said cancer,
   said method comprising at least the steps of:
a) identifying, determining genomic positions, and quantifying a total quantity n of a predefined marker of DNA structure alteration within said tumor genomic DNA sequence of the tumor genomic DNA from said patient, by comparing said tumor genomic DNA sequence with said preselected genomic DNA sequence of reference,
b) associating, to the set of preselected DNA elements, a quantity N of the predefined marker of DNA structure alteration, from the total quantity n, according to the genomic positions of the DNA elements of the set of preselected DNA elements and to the genomic positions of said predefined markers of DNA structure alteration obtained at step a),
c) computing a test score relative to a quantity N of predefined markers of DNA structure alteration associated with the length L of said set of preselected DNA elements, said test score being based on a comparison between said quantity N of predefined markers of DNA structure alteration and a reference level defined by a random model relative to the total quantity n of the predefined marker of DNA structure alteration over the length l of the preselected genomic DNA sequence of reference, and
d) comparing said test score to said at least one reference threshold in order to classify said patient.

In the method for classifying a cancer patient according to the invention, said random model used in step c) may be a random breakage model, especially using binomial, poisson, beta, normal, or power law distributions. Such a random breakage model is advantageously used to evaluate the propensity of such a DNA element to break more than expected.

In one embodiment, it is disclosed a method for determining threshold(s) representative of an evolution of a cancer, especially representative of a risk of metastasis for patients having said cancer, especially for leiomyosarcoma, especially to be used in the classification method as disclosed herein.

In embodiments, a method for determining threshold(s) may use:
i) a classifier,
ii) a plurality of tumor genomic DNA sequences obtained by sequencing a plurality of tumor genomic DNA obtained from a plurality of patients having said cancer
iii) at least one set of preselected DNA elements, each DNA element having a genomic position, said preselected set of DNA elements having a length L in base pairs,
iv) a preselected genomic DNA sequence of reference having a length l in base pairs, and
v) a plurality of scores relative to a quantity of a predefined marker of DNA structure alteration associated with the length L of said set of preselected DNA elements.
   said predefined markers of DNA structure alteration being previously quantified and their genomic positions identified within each tumor genomic DNA sequence of the tumor genomic DNA from each patient, by comparing said tumor genomic DNA sequence with said preselected genomic DNA sequence of reference, to obtain for each tumor genomic DNA sequence a total quantity n of predefined markers of DNA structure alteration for each genomic DNA sequence,
   said scores being based on a comparison between each quantity of predefined markers of DNA structure alteration and a reference level defined by a random model relative to each total quantity n of the predefined marker of DNA structure alteration over the length l of the preselected genomic DNA sequence of reference.

The method may comprise at least the steps wherein:
a) choosing, based on said scores, several predefined thresholds relative to said quantified predefined marker associated with the length L of said set of preselected DNA elements, each of said predefined thresholds separating two groups representative of a different evolution of said cancer,
b) for each of said predefined thresholds, computing with the classifier, by using an estimation method, a comparison value between two groups defined by the corresponding threshold, and
c) determining at least one reference threshold as separating two groups representative of a different evolution of said cancer by choosing a predefined threshold that has given an extremum value of said comparison values computed at step b).

In one exemplary embodiment, at step a), said several predefined thresholds may be chosen among said scores. In a variant, said several predefined thresholds may be computed based on said scores, especially by computing the median of two or more successive scores. Identical scores are advantageously considered only once.

In the method for determining threshold(s) representative of an evolution of a cancer according to the invention, said estimation method used in step b) may be the Kaplan-Meier test, as described in the article of L. Staub and al “Kaplan-Meier survival curves and the Log-Rank test”, seminar in statistics: survival analysis, March 2011. Said comparison value may be a p-value.

In one exemplary embodiment, said at least one reference threshold is determined by choosing a predefined threshold that has given a minimum value of said comparison values computed at step b). In a variant, a maximum value may be considered, especially if a log 10 of said comparison values is computed.

In one exemplary embodiment, the method as disclosed herein may be applied on groups of at least 3 patients having said cancer, better at least 7 patients.

In a preferred embodiment, two reference thresholds are determined to define three different groups representative of a different evolution of said cancer, especially three groups named low, medium, and high, each representative of a different evolution of said cancer, as will be explained further below.

Said classifier used in the method for determining threshold(s) representative of an evolution of a cancer according to the invention, called iPART for “Iterative multi-thresholds PARTitionning”, corresponds to an unsupervised decision tree (UDT).

Such a method combines properties and objectives of both unsupervised clustering and DT. Hence, iPART advantageously looks for thresholds maximizing the differences in the groups instead of computing pairwise distance and constructing hierarchical clusters. It resembles DT and regression trees (RT) by using thresholds to split groups, however it differs from DT by being unsupervised, that is to say the groups one is looking for to make the classifier learns them are unknown. It also differs from RT by not trying to predict quantitative variables. The fundamental difference with both RT and DT, is its ability to use binary or ternary modes, that is to say splitting data into two or three groups. This property of iPART allows having useful insights on the data.

It also differs from DT and RT by the use of an estimation method, especially Kaplan-Meier test, allowing to measure, for example, the fraction of patients developing metastasis after diagnosis instead of, respectively, the GINI purity or information gain indexes that need supervised data, i.e. pre-established groups, to be computed, and the sum of squared residuals between predicted and actual quantitative variables to be computed for RT.

It also resembles unsupervised machine learning, as hierarchical clustering and k-means, by aiming to find natural patterns and thus different groups in the data, but differs from it by the fact that pair-wise distances are not computed and that groups are not constructed by minimizing their intra-group variance. On the contrary, the method according to the invention iterates over all possible thresholds, find thresholds that maximizes the difference between the split groups, for example in term of the speed of metastatic events occurrence in two groups, by finding an extremum value of an estimation method, especially the Kaplan-Meier test. The invention therefore offers a way to find natural frontiers that maximize the differences between groups instead of constructing groups that minimize intra-group and overall variance.

In one exemplary embodiment, and in particular when the Kaplan-Meier test is used, clinical data associated to each patient of said plurality of patients having said cancer may be used during the learning of the classifier. In a preferred embodiment, said clinical data may be information concerning the fact that said patient had a metastasis, and the date of such metastasis. Said clinical data may also be the age and/or the gender of said patient.

In the method for determining threshold(s) representative of an evolution of a cancer according to the invention, a selection of the thresholds corresponding to comparison values lower or higher than an arbitrary comparison value may be performed to choose said predefined thresholds at step a), also named thresholds of significance.

Especially in the case where such a selection gives more than ten or fifteen thresholds, a selection of the thresholds corresponding to comparison values lower or higher than an arbitrary comparison value may be performed to shortlist said predefined thresholds of significance. An alternative approach may be choosing the extremum of n bins of thresholds listed into several bins of equal sizes, for example 10 bins, and to select the score giving an extremum comparison value in each bin.

Then, for each combination of two thresholds of a 2-combination of predefined thresholds or short-listed predefined thresholds, the classifier computes a comparison value between three groups defined by the two thresholds of each combination, by using an estimation method.

Said test score(s) and reference threshold(s) used in the methods according to the invention may be transmitted to a user by any suitable mean, for example by being displayed on a screen of an electronic device, printed, or by vocal synthesis.

Each step of the methods according to the invention may be carried out on an electronic system, in particular a personal computer, a calculation server or a medical imaging device, preferably comprising at least a microcontroller and a memory.

The features defined above for the method for classifying a cancer patient apply to the method for determining threshold(s) representative of an evolution of a cancer, and vice and versa.

The features defined above for the methods apply to the device for determining threshold(s) representative of an evolution of a cancer, as defined above.

### Computer Program Products and Devices

Such methods according to the invention are advantageously performed by means of computer programs, automatically on any electronic system comprising a processor, especially a computer.

A further object of the invention is a computer program product for classifying a cancer patient in a group representative of the evolution of said cancer, as defined above.

The invention also relates to a computer program product for determining threshold(s) representative of an evolution of a cancer, especially representative of a risk of metastasis for patients having said cancer, especially for Leiomyosarcoma, especially to be used in the classification method according to the invention, using
i) a classifier,
ii) a plurality of tumor genomic DNA sequences obtained by sequencing a plurality of tumor genomic DNA obtained from a plurality of patients having said cancer,
iii) at least one set of preselected DNA elements, each DNA element having a genomic position, said preselected set of DNA elements having a length L in base pairs,
iv) a preselected genomic DNA sequence of reference having a length l in base pairs, and
v) a plurality of scores relative to a quantity of a predefined marker of DNA structure alteration associated with the length L of said set of preselected DNA elements,
   said predefined markers of DNA structure alteration being previously quantified and their genomic positions identified within each tumor genomic DNA sequence of the tumor genomic DNA from each patient, by comparing said tumor genomic DNA sequence with said preselected genomic DNA sequence of reference, to obtain for each tumor genomic DNA sequence a total quantity n of predefined markers of DNA structure alteration for each genomic DNA sequence,
   said scores being based on a comparison between each quantity of predefined markers of DNA structure alteration and a reference level defined by a random model relative to each total quantity n of the predefined marker of DNA structure alteration over the length l of the preselected genomic DNA sequence of reference,
      the computer program product comprising a support and stored on this support instructions that can be read by a processor, these instructions being configured to:
a) choose, based on said scores, several predefined thresholds relative to said quantified predefined marker associated with the length L of said set of preselected DNA elements, each of said predefined thresholds separating two groups representative of a different evolution of said cancer,
b) for each of said predefined thresholds, compute with the classifier, by using an estimation method, a comparison value between two groups defined by the corresponding threshold, and
c) determine at least one reference threshold as separating two groups representative of a different evolution of said cancer by choosing a predefined threshold that has given an extremum value of said comparison values computed at step b).

The features defined above for the methods apply to the computer program products.

Also is disclosed a device for determining threshold(s) representative of an evolution of a cancer, especially a risk of metastasis for patients having said cancer, especially for leiomyosarcoma, and comprising a classifier.

In one exemplary embodiment, said device as disclosed herein may use:
i) a plurality of tumor genomic DNA sequences obtained by sequencing a plurality of tumor genomic DNA obtained from a plurality of patients having said cancer,
ii) at least one set of preselected DNA elements, each DNA element having a genomic position, said preselected set of DNA elements having a length L in base pairs,
iii) a preselected genomic DNA sequence of reference having a length l in base pairs, and
iv) a plurality of scores relative to a quantity of a predefined marker of DNA structure alteration associated with the length L of said set of preselected DNA elements,
   said predefined markers of DNA structure alteration being previously quantified and their genomic positions identified within each tumor genomic DNA sequence of the tumor genomic DNA from each patient, by comparing said tumor genomic DNA sequence with said preselected genomic DNA sequence of reference, to obtain for each tumor genomic DNA sequence a total quantity n of predefined markers of DNA structure alteration for each genomic DNA sequence,
   said scores being based on a comparison between each quantity of predefined markers of DNA structure alteration and a reference level defined by a random model relative to each total quantity n of the predefined marker of DNA structure alteration over the length l of the preselected genomic DNA sequence of reference.

A device as disclosed herein may be configured to:
a) choose, based on said scores, several predefined thresholds relative to said quantified predefined marker associated with the length L of said set of preselected DNA elements, each of said predefined thresholds separating two groups representative of a different evolution of said cancer,
b) for each of said predefined thresholds, compute with the classifier, by using an estimation method, a comparison value between two groups defined by the corresponding threshold, and
c) determine at least one reference threshold as separating two groups representative of a different evolution of said cancer by choosing a predefined threshold that has given an extremum value of said comparison values computed at step b).

The classifier and the estimation method may be as disclosed herein.

Also is disclosed a device for classifying a patient having a cancer in a group representative of an evolution of said cancer, especially representative of a risk of metastasis of said cancer, especially for leiomyosarcoma, the method using:
i) a tumor genomic DNA sequence obtained by sequencing a tumor genomic DNA obtained from said patient,
ii) at least one set of preselected DNA elements, each DNA element having a genomic position, said set of DNA elements having a length L in base pairs,
iii) a preselected genomic DNA sequence of reference having a length l in base pairs, and
iv) at least one reference threshold separating two groups of patients, each group being representative of a different evolution of said cancer,
   said device being configured to:
a) identify, determine genomic positions, and quantify a total quantity n of a predefined marker of DNA structure alteration within said tumor genomic DNA sequence of the tumor genomic DNA from said patient, by comparing said tumor genomic DNA sequence with said preselected genomic DNA sequence of reference,
b) associate, to the set of preselected DNA elements, a quantity N of the predefined marker of DNA structure alteration, from the total quantity n, according to the genomic positions of the DNA elements of the set of preselected DNA elements and to the genomic positions of said predefined markers of DNA structure alteration obtained at step a),
c) compute a test score relative to a quantity N of predefined markers of DNA structure alteration associated with the length L of said set of preselected DNA elements, said test score being based on a comparison between said quantity N of predefined markers of DNA structure alteration and a reference level defined by a random model relative to the total quantity n of the predefined marker of DNA structure alteration over the length l of the preselected genomic DNA sequence of reference, and
d) compare said test score to said at least one reference threshold in order to classify said patient.

### Evolution of a Cancer

In some embodiments, methods as disclosed herein may relate to diagnostic method for predicting an evolution of a cancer in a patient having said cancer.

Such a method may:
using at least a tumor genomic DNA sequence obtained by sequencing a tumor genomic DNA obtained from said patient, and
comprising at least the step of submitting said tumor genomic DNA sequence to a method as disclosed herein for classifying a cancer patient in a group representative of an evolution of said cancer, said classification being predictive of an evolution of said cancer of said patient.

A cancer evolution may be the occurrence of metastasis, a responsiveness of cancer to a treatment, the occurrence of patient's demise within a certain predefined period of time, or the instability of the genome of cancer cells.

A diagnostic method as disclosed herein may be for predicting (i) a risk of metastasis of said cancer, (ii) a sensibility of said cancer to a cancer treatment, (iii) a risk of mortality of said patient to said cancer, or (iv) a genome instability of genome of cancer cells of said cancer of said patient.

Herein, the term “risk” used with regard to a certain event intends to refer to a likelihood of occurrence of this event in a given cancer patient. For instance, a risk of metastasis refers to a likelihood of occurrence of metastasis in a given cancer patient.

In one exemplary embodiment, a cancer patient concerned by a diagnostic method as disclosed herein may be a patient having a sarcoma, as for example a leiomyosarcoma.

In one exemplary embodiment, a diagnostic method as disclosed herein may be for predicting a risk of metastasis of said cancer. “Metastasis” refers to the spread of cancer cells from the place where they first formed to another part of the body. In metastasis, cancer cells break away from the original (primary) tumor, travel through the blood or lymph system, and form a new tumor in other organs or tissues of the body. The new, metastatic tumor is the same type of cancer as the primary tumor. For example, if breast cancer spreads to the lung, the cancer cells in the lung are breast cancer cells, not lung cancer cells.

In such diagnostic method, the test score computed for the cancer patient may be compared to a reference threshold. The reference threshold may be obtained by carrying out the disclosed methods described herein for classifying cancer patients and determining threshold(s) representative of an evolution of a cancer on several isolated genomic DNA isolated from several cancer patient, such at least 3 or at least 7 cancer patients. A test score may be the −log 10 of the probability (p) of random distribution of the quantified predefined marker of DNA structure alteration computed using the RBM as disclosed herein. A reference threshold separates two group of patients having distinct cancer evolution, such as different risk of metastasis occurrence.

The test score and threshold reference may be determined using a set of preselected of DNA elements consisting in transcription-associated chromosomal instability elements, in replication-associated chromosomal instability elements, or in a combination of transcription-associated chromosomal instability elements and replication-associated chromosomal instability elements.

For example, for leiomyosarcoma, one may use at least two threshold references to classify a patient in a group of either low, medium, or high probability of DNA alteration in a set of preselected DNA elements. The group of medium probability of DNA alteration may define a group of high risk of metastasis occurrence. While the groups of low or high probability of DNA alteration may define groups of low risk of metastasis occurrence.

A reference threshold may be defined with respect to a set of preselected DNA elements which may be either TRACi (or iTRAC) or RACINi (or iRACIN), as disclosed herein.

A reference threshold defined with respect to TRACi (or iTRAC) and computed as a −log 10 of the probability (p) of random distribution of the quantified predefined marker of DNA structure alteration computed using the RBM as disclosed herein, and separating a group of low probability and a group of medium probability of DNA alterations in TRACe may be from about 0.50 to about 1.50, for example may be from about 0.70 to about 1.30, and for example may be about 0.99 (or 1.00).

A reference threshold defined with respect to TRACi (or iTRAC) and computed as a −log 10 of the probability (p) of random distribution of the quantified predefined marker of DNA structure alteration computed using the RBM as disclosed herein, and separating a group of medium probability and a group of high probability of DNA alterations may be from in TRACe about 1.70 to about 2.80, for example may be from about 2.00 to about 2.50, and for example may be about 2.29 (or 2.30).

A reference threshold defined with respect to RACINi (or iRACIN) and computed as a −log 10 of the probability (p) of random distribution of the quantified predefined marker of DNA structure alteration computed using the RBM when said probability is a one-sided p-value wherein only the values superior to said reference level are taken into account, and separating a group of low probability and a group of medium probability of DNA alterations in RACINe may be from about 0.45 to about 1.05, for example may be from about 0.65 to about 0.85, and for example may be about 0.74 (or 0.75).

A reference threshold defined with respect to RACINi (or iRACIN) and computed as a −log 10 of the probability (p) of random distribution of the quantified predefined marker of DNA structure alteration computed using the RBM when said probability is a one-sided p-value wherein only the values superior to said reference level are taken into account, and separating a group of medium probability and a group of high probability of DNA alterations in RACINe may be from about 1.15 to about 1.45, for example may be from about 1.20 to about 1.40, and for example may be about 1.30.

Depending on the classification, a differentiated treatment can be proposed to the cancer patient, such as surgery, chemotherapy, radiotherapy, immunotherapy, targeted therapy, and combinations thereof.

In one embodiment, a diagnostic method as disclosed herein may be used for predicting a sensibility of a cancer from a cancer patient to a cancer treatment.

In such diagnostic method, the test score computed for the cancer patient may be compared to one, two, or more, reference thresholds. The reference threshold(s) may be obtained by carrying out the disclosed methods described herein for classifying cancer patients and determining threshold(s) representative of a distribution of a quantity of predefined markers of DNA structure alteration in a set of preselected DNA elements, which in turn is representative of an evolution of a cancer on several isolated genomic DNA isolated from several cancer patient, such at least 3 or at least 7 cancer patients. A test score may be the −log 10 of the probability (p) of random distribution of the quantified predefined marker of DNA structure alteration computed using the RBM as disclosed herein. With such one, two, or more, reference thresholds, a cancer patient with a test score below the threshold or the lower threshold may be classified having poor prognosis after receiving chemotherapy treatment, while the patients classified above the threshold, or the medium or high threshold are classified as having no therapeutic benefices further to a chemotherapy treatment. It may also be classified to having a therapeutic benefice

Depending on the classification of the cancer of a cancer patient, a cancer treatment may be considered as providing no-therapeutic benefice to the cancer patient and therefore it may be proposed to the patient to not undergo such treatment. Conversely, depending on the classification a cancer treatment may be considered as providing good therapeutic benefice to the cancer patient and therefore should be proposed to the patient.

In one exemplary embodiment, a diagnostic method as disclosed herein may be for predicting a risk of metastasis of a cancer or a sensibility of a cancer to a cancer treatment and the set of preselected of DNA elements consists in transcription-associated chromosomal instability elements, in replication-associated chromosomal instability elements, or in a combination of transcription-associated chromosomal instability elements and replication-associated chromosomal instability elements.

For example, for leiomyosarcoma, one may use at least one, two, or more threshold references to classify a patient in a group of low, medium, or high probability of DNA alteration in a set of preselected DNA elements, which in turn are representative of cancer sensibility to a cancer treatment.

In case of the use of one threshold of reference, the group of low probability of DNA alteration may define a group for which chemotherapeutic treatment may be detrimental, and the group of high probability of DNA alteration may define a group for which beneficial effect may occur as a result of a chemotherapeutic treatment.

In case of the use of two threshold of references, the group of low probability of DNA alteration may define a group for which chemotherapeutic treatment may be detrimental, the group of medium probability of DNA alteration may define a group for which no beneficial effect may occur as a result of a chemotherapeutic treatment, and the group of high probability of DNA alteration may define a group for which beneficial effect may occur as a result of a chemotherapeutic treatment.

The values of reference thresholds for low, medium, and high probability of DNA alterations for leiomyosarcoma may be as disclosed herein for the risk of metastasis occurrence.

In one exemplary embodiment, a diagnostic method as disclosed herein may be for identifying a genome instability mechanism of a cancer in a patient having said cancer.

The method may:
use at least a tumor genomic DNA sequence obtained by sequencing a tumor genomic DNA obtained from said patient, and
   comprise at least the step of submitting said tumor genomic DNA sequence to the method according to the invention for classifying a cancer patient in a group representative of a genome instability mechanism being either a replication-associated chromosomal instability or a be transcription-associated chromosomal instability.

Knowing the mechanism underlying the genome instability, it is possible to stratify cancer patients according to the mechanism and to propose a therapeutic cancer treatment directed to the mechanism. Genome instability may be dominated either by a replication-associated chromosomal instability or by a transcription-associated chromosomal instability or may be a mix of replication-associated and transcription-associated chromosomal instabilities. A cancer resulting mainly from transcription-associated chromosomal instability may be more sensitive to cancer therapeutic treatment targeting DNA transcription process. A cancer resulting mainly from replication-associated chromosomal instability may be more sensitive to cancer therapeutic treatment targeting DNA replication process.

In one embodiment, a test score is obtained with respect to a set of preselected DNA elements representative of transcription-associated chromosomal elements. The test score may be then compared to reference thresholds allowing to stratify patients in groups of low, medium or high transcription-associated chromosomal instability.

In one embodiment, a test score is obtained with respect to a set of preselected DNA elements representative of replication-associated chromosomal elements. The test score may be then compared to reference thresholds allowing to stratify patients in groups of low, medium or high replication-associated chromosomal instability.

In another embodiment, test scores are obtained with respect to a set of preselected DNA elements representative of replication-associated chromosomal elements and with respect to a set of preselected DNA elements representative of transcription-associated chromosomal elements. The test scores may be then compared to reference thresholds allowing to stratify patients in groups of low, medium or high replication-associated chromosomal instability and in groups of low, medium or high transcription-associated chromosomal instability.

The classifying of a patient according to genome instability allows to propose a cancer therapeutic treatment adapted to the mechanisms underlying the occurrence of the cancer as it is admitted that type and degree of genome instability may affect sensibility of a cancer patient to a given cancer therapeutic treatment.

In one embodiment, in designing clinical trial, the classifying of patients in different groups according to the genome instability allows defining homogeneous groups of patients and to assess efficacy of the tested cancer therapeutic according to specific type and degree of genome instability. It is then possible to assess in which a type and degree of genome instability may affect efficacy of a given cancer therapeutic treatment.

In one exemplary embodiment, a diagnostic method as disclosed herein may be a for predicting a risk or mortality, or chance of survival, of said patient cancer.

The risk of mortality may be defined as a risk of occurrence of a cancer patient's demise with a certain period of time as a consequence of the cancer of the patient. It reflects the chance of survival of a cancer patient within a certain period of time. Starting from time or diagnosis as carried out according to methods as disclosed herein, the period of time extends from present to some point of time in the future. The period of time may last from weeks to years, and for example may be of about 1, 2, 3, 4, 5, 6, 7, 8, 9, 10, 11 or 12 months, or 1, 2, 3, 4, 5, 6, 7, 8, 9, 10 years or more after the diagnosis is made.

In such diagnostic method, the test score computed for the cancer patient may be compared to several reference thresholds. The reference thresholds may be obtained as disclosed herein. The plurality of reference thresholds will define a plurality of group of patients, each characterized by a certain period of time of survival for certain period of time or a risk of mortality within a certain period of time. The comparison of the test score of the cancer patient with the plurality of reference thresholds allows classifying the cancer patient within a given group of patients, and will allow predict the survival time of the cancer patient, or his or her risk of mortality within a certain period of time.

### Methods for Diagnosing and Treating a Cancer Patient

In some embodiments, a method as disclosed herein may be for selecting a treatment of a cancer for a patient having said cancer and for treating said patient. The method may:
use at least a tumor genomic DNA sequence obtained by sequencing a tumor genomic DNA obtained from isolated cancer cells of said patient, and
comprise at least the steps of:
   submitting said at least tumor genomic DNA sequence to the method as disclosed herein for classifying said cancer of said patient in a group associating said cancer with at least one cancer treatment, and
   treating said patient with said at least one cancer treatment associated with said group.

In such embodiment, the test score of the cancer patient may be compared to a plurality of reference thresholds, each threshold separating two groups of patients, each group being characterized as being responsive to a cancer treatment or to a plurality of cancer treatments.

In some embodiments, a method as disclosed herein may be for predicting a sensibility of a cancer in a patient having said cancer to a cancer treatment and for treating said patient, said method:
using at least a tumor genomic DNA sequence obtained by sequencing a tumor genomic DNA obtained from isolated cancer cells of said patient, and
the method comprising at least the steps of:
   submitting said at least tumor genomic DNA sequence to the method according to the invention for classifying said cancer of said patient in a group representative of a sensibility of said cancer to a cancer treatment, said classification being predictive of a sensibility of said cancer of said patient to said cancer treatment, and
   treating said patient with said cancer treatment.

The used treatment is a treatment for which the classification of the cancer has classified the tested cancer patient in a group of cancer considered as being sensitive and responsive to this treatment.

In such embodiment, the test score of the cancer patient may be compared to a reference threshold, separating two groups of patients, one being characterized as being responsive to the cancer treatment and the other being non-responsive to the cancer treatments.

In one embodiment, a cancer patient may be a patient having a sarcoma.

A sarcoma may be an undifferentiated pleomorphic sarcoma, a liposarcoma, a rhabdomyosarcoma, an angiosarcoma from blood vessels, a malignant peripheral nerve sheath tumor (MPNST or PNST), a gastrointestinal stromal tumor sarcoma (GIST), a synovial sarcoma, a dermatofibrosarcoma, a fibrohistiocytic sarcoma, a myxofibrosarcoma, a Kaposi sarcoma, a chondro-osseous sarcomas, a leiomyosarcoma, or any other subtype of sarcoma.

In one exemplary embodiment, a cancer may be a leiomyosarcoma.

The methods as disclosed herein may be repeated overtime, at least one, 2, 3, 4, 5 or more time, over the duration of cancer treatment to assess whether the selected treatment still presents some benefits for the cancer patient or whether the sensibility of the cancer of the cancer patient to a treatment has evolve towards an increase or decrease of sensibility in order to reevaluate the benefit of the proposed cancer treatment.

A cancer treatment which may be considered may a surgical resection of the cancer or tumor.

In one embodiment, a cancer treatment may be a surgical treatment, a chemotherapy, a radiotherapy, an immunotherapy, a targeted therapy, or a combination thereof.

In some embodiment, a suitable treatment may be to propose no treatment.

In one embodiment, a cancer treatment is not surgical treatment.

In one embodiment, a cancer treatment may be a chemotherapy, a radiotherapy, an immunotherapy, a targeted therapy, or a combination thereof.

In one embodiment, a cancer treatment may be a chemotherapy, an immunotherapy, a targeted therapy, or a combination thereof, or any 2-combination or 3-combination thereof.

As exemplary of suitable chemotherapy, one may mention alkylating agents, such as altretamine, bendamustine, busulfan, carboplatin, carmustine, chlorambucil, cisplatin, cyclophosphamide, dacarbazine, ifosfamide, lomustine, mechlorethamine, melphalan, oxaliplatin, temozolomide, thiotepa, trabectedin; nitrosoureas such as carmustine, lomustine, streptozocin; antimetabolites, such as azacitidine, 5-fluorouracil (5-fu), 6-mercaptopurine (6-mp), capecitabine (xeloda), cladribine, clofarabine, cytarabine (ara-c), decitabine, floxuridine, fludarabine, gemcitabine (gemzar), hydroxyurea, methotrexate, nelarabine, pemetrexed (alimta), pentostatin, pralatrexate, thioguanine, trifluridine/tipiracil combination, anti-tumor antibiotics, such as daunorubicin, doxorubicin (adriamycin), doxorubicin liposomal, epirubicin, idarubicin, valrubicin, bleomycin, dactinomycin, mitomycin-c, mitoxantrone; topoisomerase inhibitors, such as irinotecan, irinotecan liposomal, topotecan, etoposide (vp-16) mitoxantrone, teniposide; mitotic inhibitors, such as cabazitaxel, docetaxel, nab-paclitaxel, paclitaxel, vinblastine, vincristine, vincristine liposomal, vinorelbine; corticosteroids, such as prednisone, methylprednisolone, dexamethasone; or other chemotherapy drugs, such as all-trans-retinoic acid, arsenic trioxide, asparaginase, eribulin, hydroxyurea, ixabepilone, mitotane, omacetaxine, pegaspargase, procarbazine, romidepsin, vorinostat.

As exemplary of suitable immunotherapy, one may mention monoclonal antibodies and tumor-agnostic treatments, such as checkpoint inhibitors; oncolytic virus therapy; t-cell therapy; cancer vaccines.

As exemplary of suitable radiotherapy, one may mention external beam radiation therapy and internal radiation therapy.

In one exemplary embodiment, a cancer treatment for sarcoma may be dactinomycin, doxorubicin hydrochloride, eribulin mesylate, imatinib mesylate, pazopanib hydrochloride, tazemetostat hydrobromide, or trabectedin.

### Method for Selecting a DNA Element as a Biomarker of an Evolution of a Cancer

In one embodiment, a method as disclosed herein may be used for selecting a DNA element as a biomarker of an evolution of a cancer in a patient having said cancer.

As exemplary embodiment, the method may use:
i) a tumor genomic DNA sequence obtained by sequencing a tumor genomic DNA obtained from said patient,
ii) a plurality of preselected DNA elements, each DNA element having a genomic position, a length le in base pairs,
iii) a preselected genomic DNA sequence of reference having a length l in base pairs, and
iv) a preselected threshold or a set of preselected thresholds for selecting said DNA element to be used as a biomarker,

The method may comprise at least the steps of:
a) identifying, determining genomic positions, and quantifying a total quantity n of a predefined marker of DNA structure alteration within said tumor genomic DNA sequence of the tumor genomic DNA from said patient, by comparing said tumor genomic DNA sequence with said preselected genomic DNA sequence of reference,
b) associating, to each DNA element of the plurality of DNA elements, a quantity Ne of the predefined marker of DNA structure alteration, from the total quantity n, according to the genomic positions of said DNA element and to the genomic positions of said predefined markers of DNA structure alteration obtained at step b),
c) computing for each DNA element,
   a test score relative to said quantity Ne of predefined markers of DNA structure alteration associated with the length le of said DNA element,
   said test score based on a comparison between said quantity Ne of predefined markers of DNA structure alteration and a reference level defined by a random model relative to the total quantity n of the predefined marker of DNA structure alteration over the length l of the preselected genomic DNA sequence of reference, and
d) comparing said test scores to said preselected threshold or said set of preselected thresholds to select said DNA element as a biomarker of an evolution of a cancer in a patient having said cancer.

In one embodiment, a test score may be as disclosed herein and may use a probability p of having a quantity N of predefined markers of DNA alteration within a length le of a DNA element, and a probability pᵤ which may be a ratio of the total quantity n of identified and quantified predefined markers of DNA structure alteration to the length l of the preselected genomic DNA sequence of reference.

In some embodiment, a test score may be a biomarker.

A test score, or Hscore in the Examples section, may be computed as being the −log 10 of the probability (p) computed as disclosed herein. This test score or Hscore translates the predefined marker hotspotness magnitude scale for the propensity of the considered DNA elements to be more altered, or to break more, than expected by chance under RBM.

For example, the test score or Hscore threshold to retain a DNA element as a hotspot may be settled as superior or equal to 3. Then, hotspots may be defined as follows: 1) DNA elements significantly more broken than expected by chance (Hscore ≥3).

The method may be repeated on a plurality of DNA elements. The DNA elements retained as biomarker may be used in a set of preselected DNA elements. The set of preselected DNA elements in turn may define a biomarker.

### Cancer

In one embodiment, a cancer to which method as disclosed herein may be applied may be a cancer selected among leukemias, lymphomas, carcinomas, melanomas, and sarcomas.

In one embodiment, a cancer may be a cancer selected among acute myeloid leukemia (LAML or AML), acute lymphoblastic leukemia (ALL), adrenocortical carcinoma (ACC), bladder urothelial cancer (BLCA), brain stem glioma, brain lower grade glioma (LGG), brain tumor, breast cancer (BRCA), bronchial tumors, Burkitt lymphoma, cancer of unknown primary site, carcinoid tumor, carcinoma of unknown primary site, central nervous system atypical teratoid/rhabdoid tumor, central nervous system embryonal tumors, cervical squamous cell carcinoma, endocervical adenocarcinoma (CESC) cancer, childhood cancers, cholangiocarcinoma (CHOL), chordoma, chronic lymphocytic leukemia, chronic myelogenous leukemia, chronic myeloproliferative disorders, colon (adenocarcinoma) cancer (COAD), colorectal cancer, craniopharyngioma, cutaneous T-cell lymphoma, endocrine pancreas islet cell tumors, endometrial cancer, ependymoblastoma, ependymoma, esophageal cancer (ESCA), esthesioneuroblastoma, Ewing sarcoma, extracranial germ cell tumor, extragonadal germ cell tumor, extrahepatic bile duct cancer, gallbladder cancer, gastric (stomach) cancer, gastrointestinal carcinoid tumor, gastrointestinal stromal cell tumor, gastrointestinal stromal tumor (GIST), gestational trophoblastic tumor, glioblastoma multiforme glioma GBM), hairy cell leukemia, head and neck cancer (HNSD), heart cancer, Hodgkin lymphoma, hypopharyngeal cancer, intraocular melanoma, islet cell tumors, Kaposi sarcoma, kidney cancer, Langerhans cell histiocytosis, laryngeal cancer, lip cancer, liver cancer, Lymphoid Neoplasm Diffuse Large B-cell Lymphoma [DLBCL), malignant fibrous histiocytoma bone cancer, medulloblastoma, medullo epithelioma, melanoma, Merkel cell carcinoma, Merkel cell skin carcinoma, mesothelioma (MESO), metastatic squamous neck cancer with occult primary, mouth cancer, multiple endocrine neoplasia syndromes, multiple myeloma, multiple myeloma/plasma cell neoplasm, mycosis fungoides, myelodysplastic syndromes, myeloproliferative neoplasms, nasal cavity cancer, nasopharyngeal cancer, neuroblastoma, Non-Hodgkin lymphoma, nonmelanoma skin cancer, non-small cell lung cancer, oral cancer, oral cavity cancer, oropharyngeal cancer, osteosarcoma, other brain and spinal cord tumors, ovarian cancer, ovarian epithelial cancer, ovarian germ cell tumor, ovarian low malignant potential tumor, pancreatic cancer, papillomatosis, paranasal sinus cancer, parathyroid cancer, pelvic cancer, penile cancer, pharyngeal cancer, pheochromocytoma and paraganglioma (PCPG), pineal parenchymal tumors of intermediate differentiation, pineoblastoma, pituitary tumor, plasma cell neoplasm/multiple myeloma, pleuropulmonary blastoma, primary central nervous system (CNS) lymphoma, primary hepatocellular liver cancer, prostate cancer such as prostate adenocarcinoma (PRAD), rectal cancer, renal cancer, renal cell (kidney) cancer, renal cell cancer, respiratory tract cancer, retinoblastoma, rhabdomyosarcoma, salivary gland cancer, sarcoma (SARC), Sezary syndrome, skin cutaneous melanoma (SKCM), small cell lung cancer, small intestine cancer, soft tissue sarcoma, squamous cell carcinoma, squamous neck cancer, stomach (gastric) cancer, supratentorial primitive neuroectodermal tumors, T-cell lymphoma, testicular cancer testicular germ cell tumors (TGCT), throat cancer, thymic carcinoma, thymoma (THYM), thyroid cancer (THCA), transitional cell cancer, transitional cell cancer of the renal pelvis and ureter, trophoblastic tumor, ureter cancer, urethral cancer, uterine cancer, uterine cancer, uveal melanoma (UVM), vaginal cancer, vulvar cancer, Waldenstrom macroglobulinemia, or Wilm's tumor.

In an exemplary embodiment, a cancer may be a sarcoma. A sarcoma may be an undifferentiated pleomorphic sarcoma, a liposarcoma, a rhabdomyosarcoma, an angiosarcoma from blood vessels, a malignant peripheral nerve sheath tumor (MPNST or PNST), a gastrointestinal stromal tumor sarcoma (GIST), a synovial sarcoma, a dermatofibrosarcoma, a fibrohistiocytic sarcoma, a myxofibrosarcoma, a Kaposi sarcoma, a chondro-osseous sarcomas, a leiomyosarcoma, or any other subtype of sarcoma.

In an exemplary embodiment, a cancer may be a rhabdomyosarcoma.

In an exemplary embodiment, a cancer may be a myxofibrosarcoma.

In an exemplary embodiment, a cancer may be a leiomyosarcoma.

It is to be understood that the disclosure encompasses all variations, combinations, and permutations in which at least one limitation, element, clause, descriptive term, etc., from at least one of the listed claims is introduced into another claim dependent on the same base claim (or, as relevant, any other claim) unless otherwise indicated or unless it would be evident to one of ordinary skill in the art that a contradiction or inconsistency would arise. Where elements are presented as lists, e.g., in Markush group or similar format, it is to be understood that each subgroup of the elements is also disclosed, and any element(s) can be removed from the group. It should be understood that, in general, where the disclosure, or aspects of the disclosure, is/are referred to as comprising particular elements, features, etc., they also encompass embodiments consisting, or consisting essentially of, such elements, features, etc. For purposes of simplicity those embodiments have not in every case been specifically set forth in so many words herein. It should also be understood that any embodiment or aspect of the disclosure can be explicitly excluded from the claims, regardless of whether the specific exclusion is recited in the specification. The publications and other reference materials referenced herein to describe the background of the disclosure and to provide additional detail regarding its practice are hereby incorporated by reference.

The following examples are provided for purpose of illustration and not limitation.

### EXAMPLES

### Example 1: Materials and Methods

### Samples

Samples (112) used in this study were collected as part of the ICGC program (International Cancer Genome Consortium), with patient consent. Samples were frozen tissues provided by pathologists and a blood sample for each included patient provided by medical oncologists.

All cases have been systematically reviewed by expert pathologists of the French Sarcoma Group according to the World Health Organization (Fletcher, C., Bridge, J. A., Hogendoorn, P. & Mertens, F. WHO Classification of Tumors of Soft Tissue and Bone. vol. 5 (IARC Press, 2013).

### DNA Extraction

Genomic DNA from frozen samples was isolated using standard phenol-chloroform extraction protocol (Chomeczynski et Sacchi, Analytical Biochemistry 162 (1): 156-59, 1987). DNA was quantified using Nanodrop 1000 spectrophotometer according to manufacturer recommendations (Thermo Scientific, Waltham, MA, USA). Blood material from included patients was also available. Genomic DNA from blood samples was extracted using customized automated purification of DNA from compromised blood samples on the Autopure LS protocol according to the manufacturer's recommendations (Qiagen, Hilden, Germany) with increased centrifugation of 10 min for DNA precipitation and DNA wash.

### Whole Genome Sequencing and Analysis

To construct short-insert paired-end libraries a no-PCR protocol was used with the TruSeq™DNA Sample Preparation Kit v2 (Illumina Inc., San Diego, CA, USA) and the KAPA Library Preparation kit (Kapa Biosystems, Basel, Switzerland). Briefly, 2 μg of genomic DNA were sheared on a Covaris™ E220, size selected and concentrated using AMPure XP beads (Agencourt, Beckman Coulter, Brea, CA, USA) in order to reach a fragment size of 220-480 bp. Fragmented DNA was end-repaired, adenylated and ligated to Illumina specific indexed paired-end adapters.

DNA sequencing was performed in paired-end mode, in lanes of HiSeq2000 flowcell v3 (2×100 bp) or flowcell v4 (2×125 bp) or in sequencing lanes of NovaSeq 6000 flowcell S4 (2×150 bp) (Illumina Inc., San Diego, CA, USA) to analyze tumor or matched normal/constitutive blood samples from the same patient and to reach minimal yield of 145 or 85 Gb, respectively. Two tumor samples (LMS2T and LMS5T) were sequenced in 20 lanes of HiSeq2000 flowcell v3 to reach a minimal yield of 560 Gb. Images analysis, base calling and quality scoring of the run were processed using the manufacturer's software Real Time Analysis (RTA 1.13.48) and followed by generation of FASTQ sequence files by CASAVA (Illumina Inc., San Diego, CA, USA).

DNA reads were trimmed of the 5′ and 3′ low quality bases (PHRED cut-off 20, maximum trimmed size: 30 nucleotides (nt)) and sequencing adapters were removed with Sickle2 (Joshi N A, Fass J N, 2011, “Sickle: A sliding-window, adaptive, quality-based trimming tool for FastQ files (Software Version 1.33) and SeqPrep3 (J. St. John, SeqPrep, 2011, available at github.com/jstjohn/SeqPrep), respectively. Then, DNA curated sequences were aligned using bwa v-0.7.15 (H. Li t Durbin, 2009), with default parameters, on the Human Genome version hg38 (Schneider et al., 2017). Thus, aligned reads were filtered out if their alignment score was less than 20 or if they were duplicated PCR reads, with SAMtools v1.3.1 (Heng Li et al, Bioinformatics (Oxford, England) 25 (16): 2078-79, 2009) and PicardTools v2.18.2 (“Picard Toolkit”, 2019, Broad Institute, GitHub Repository), respectively.

### Random Breakage Model, Hscore, Readable Genome Size

The readable genome is represented as a single interval of length (L in bases pairs (bp)). The uniform probability Pu of any genomic position to carry a breakpoint (BP) is the total number of BP (n) divided by L: Pu=n/L. For a given genomic interval of size (Li) and number of BP (ni), its probability to harbor ni BP under RBM is computed by the probability mass function of binomial function as:

$$f ( x ) = ( \begin{matrix}n \\ x\end{matrix} ) {{p}^{x} ( 1 - p )}^{( n - x )} ;$$

where x=ni, n=Li, p=Pu. The probability of observing more than ni BP under RBM is defined as P(X>ni)=1−P(X<=ni)=1−Σᵢ₌₀ⁿf(i), where n=ni and X is the random variable accounting for the number of observed BP over a given genomic interval.

For each DNA element's bed file, overlapping intervals were merged by using bedtools (bedtools.readthedocs.io/en/latest/index.html #) and then BP number (ni) and interval size (Li) were computed as follows: ni was computed as the sum of BP in all the merged intervals and Li was computed as the sum of all merged intervals sizes.

As said test score, an Hscore is computed as the −log 10 of P(X>ni), the probability computed using the RBM. This Hscore translates the BP hotspotness magnitude scale for the propensity of the considered DNA elements to break more than expected by chance under RBM.

“Readable genome size” has to be understood as the total ungapped length as defined at www.ncbi.nlm.nih.gov/assembly/GCF_000001405.26/ and is equal to 2,948,611,470 bp.

### Breakpoints Identification/Structural Variations Detection

Structural variants (SV) were detected from paired tumor/normal whole genome high-quality sequencing data. Paired-end reads were aligned using Bowtie v2.2.1.0 (Langmead et al. 2009) with a very sensitive local option allowing soft-clipped sequences. The algorithm has three main steps:
i) identification of potential breakpoints,
ii) characterization of the second side of the breakpoints, and
iii) selection of high-confidence breakpoints.

All parameters were set to analyze 60× tumor and 30× normal sequencing depth. Very conservative filters were used to minimize false positive detection.
i) Identification: at this step, reads with at least one soft-clipped end were analyzed as singletons. A position was considered as a potential breakpoint if it was covered by at least 4 soft-clipped reads, 5 soft-clipped bases (with at least two occurrences of two different bases), and if they represented more than 5% of the total amount of reads at this position in the tumor sample. Potential somatic events were selected by discarding positions covered by at least 1 read and 1 base in a surrounding 5-nucleotide window in the normal sample. They were referred to as the “first side” of the breakpoint.
ii) Characterization: to determine the genomic positions of the soft-clipped sequence from selected reads, the UCSC blat server was used (Kent 2002). If no match was returned, the reverse complement sequence was pulled to test. If there was still no match, the BAM file was investigated for some soft-clip somatic position around the discordant or oversized-insert read mate (hereafter named abnormal) location from the first side of the breakpoint. Because of the small size of the soft-clipped sequence, multiple matches can be found. Soft-clipped abnormal read mates were used to select matches with the most coherent chromosomic locations. They were referred to as the “second side” of the breakpoint.
iii) Selection: Positions detected from both the first and second sides (in a 5-nucleotide window) were defined as the common pool. They were considered as artifacts (due to repeat regions for instance) couples of positions covered with reads and associated soft-clipped sequences separated by fewer than 15 nucleotides and they were discarded. The breakpoints were classified in three groups: high-confidence breakpoints, breakpoints needing investigation, and unique position breakpoints. If a breakpoint was covered by reads and associated soft-clipped sequences having both positions belonging to the common pool, it was classified in the first group. If a breakpoint was covered by reads and associated soft-clipped sequences having only one of the positions belonging to the common pool, it was classified in the second group. Then the missing position was searched among the filtered positions. If it was present in the normal sample, the position was discarded, and the breakpoint was completed otherwise. Finally, the third group corresponds to breakpoints with both sides outside the common pool and considered as unique: these were discarded. The sides of breakpoints were sorted according to their chromosomic positions to avoid duplicates.

### DNA Elements and Data Collection

The following DNA elements were considered in the present analysis: DNA repeats comprising MicroSatellite (MS), Simple Repeats (SR), Low Complexity (LC), Self Chains segments (SCS) which were classified into self-chains segments self-aligned (SCS-S) and self-chains segments gaped (SCS-G), Long Terminal Repeats (LTR), and Retro Transposons (RT); Non-B DNA comprising A-Phased Repeats (APR), Direct Repeats (DR), G-quadruplex (GQ), Inverted Repeats (IR), Mirror Repeats (MR), Short Tandem Repeats (STR), Z-DNA (Z) and R-Loops Forming Sequences (RLFS); and Regulatory DNA elements comprising CpG islands (CpGi), cis-regulatory modules (CRM), DNase I hypersensitive site (DHS) of promoter type (DHS_prom), DHS of enhancer type (DHS_enh), DHS of dyadic type (both enhancer and promoter signatures) (DHS_dyadic), and of DHS of other types (DHS_rest).

Data for CpG islands, microsatellites, simple repeats, low complexity, retro-transposons, long terminal repeats, self-chains segments and sequencing gaps were obtained from the UCSC Genome Browser website (genome-euro.ucsc.edu/; genome assembly hg38). All Non-B DNA but RLFS were generated using non-B DNA research tool of non-B DNA database (Cer et al. 2012). RLFS data were generated using QmRLFS-finder (Jenjaroenpun, P. et al). CRM data were obtained from Remap2018 (Cheneby et al. 2018) and data were downloaded from (pedagogix-tagc.univ-mrs.fr/remap/). DNase I-accessible regulatory regions (with −log 10(p)>=2) were downloaded from roadmap epigenomics project at personal.broadinstitute.org/meuleman/reg2map/HoneyBadger2_release/and coordinates were converted from genome assembly hg19 to hg38 using the UCSC coordinates conversion tool liftover (Kuhn, Haussler, et Kent 2013).

### TRAC Index/RACIN Index

The DNA elements were sorted in separate indexes depending whether their enrichment in BP was dependent or not of their presence inside or outside the genes (see section below Ingene/outgene split). DNA elements enriched in BP independently of their position inside or outside the genes were sorted as Replication-Associated Chromosomal INstability elements (RACINe). DNA elements enriched in BP according to their position inside the genes were sorted as Transcription-Associated Chromosomal instability elements (TRACe). For each index, all bed files of the DNA elements that belong to it into one file were pooled, and sorted according to intervals positions (bedtools), merged (bedtools) all overlapping intervals to obtain the corresponding index TRACi (or iTRAC) and RACINi (or iRACIN) as single bed file each (bedtools.readthedocs.io/en/latest/index.html #). For each index, then BP counts and interval sizes were computed, from which and Hscores under RBM were computed.

### Sliding Windows

For each DNA element (sliding window 0), each genomic feature was shifted (bedtools) by 100% of its length on the positive (+) DNA strand (sliding window+1) and on the negative (−) DNA strand (sliding window −1) and a Hscore was computed. This procedure was repeated by shifting each feature ±2×100% (sliding window+2, sliding window−2), ±3×100%, until ±8 100%.

### Ingene/Outgene Split

A DNA element was considered as inside a gene if its overlaps with at least 1 bp with the gene interval delimited by its Transcription Start Site (TSS) and Transcription End Site (TTS). Genes coordinates were taken from curated RefSeq entries from UCSC table browser page (genome.ucsc.edu/cgi-bin/hgTables; group=genes and genes_prediction:track=NCBI_RefSeq; table=RefSeq_Curated). Only genes that had expression data in those tumors were considered.

### SCS-G/SCS-S

Self-chains segments (SCS) were prepared as in the article Zhou et al., 2013, but with small variations. SC segments (SCS) were split into self-aligned (SCS-S) and gapped (SCS-G), that is having spacing intervals separating each pair of SC. SCS are defined as the segment of any paired SCS in the same chromosome and their spacing gap. The paired SCS located in different chromosomes and those in the same chromosome but having long spacing intervals (SCS size 30 kb) were filtered out to account only for local interactions. In addition, any SCS-S/SCS-G overlapping with the human genome gaps, segmental duplications (SDs) was further filtered out.

### Statistical Analysis

All statistical tests were carried out using R (R Core Team, 2020, R: A language and environment for statistical computing, R Foundation for Statistical Computing, Vienna, Austria, www.r-project.org/index.html)).

iPART

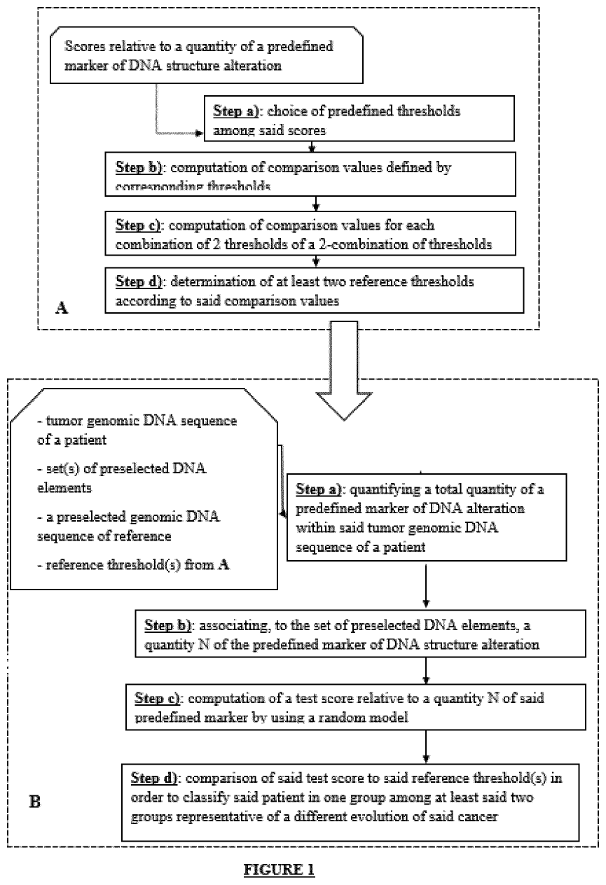

Part A of FIG. 1 is a diagram showing steps of a method for determining threshold(s) representative of an evolution of a cancer according to the invention. Said method uses a classifier, a plurality of tumor genomic DNA sequences obtained by sequencing a plurality of tumor genomic DNA obtained from a plurality of patients having said cancer, at least one set of preselected DNA elements, each DNA element having a genomic position, said preselected set of DNA elements having a length L in base pairs, a preselected genomic DNA sequence of reference having a length l in base pairs, and a plurality of scores relative to a quantity of a predefined marker of DNA structure alteration associated with the length L of said set of preselected DNA elements. Said predefined markers of DNA structure alteration are previously quantified and their genomic positions identified within each tumor genomic DNA sequence of the tumor genomic DNA from each patient, by comparing said tumor genomic DNA sequence with said preselected genomic DNA sequence of reference, to obtain for each tumor genomic DNA sequence a total quantity n of predefined markers of DNA structure alteration for each genomic DNA sequence. Said scores are based on a comparison between each quantity of predefined markers of DNA structure alteration and a reference level defined by a random model relative to each total quantity n of the predefined marker of DNA structure alteration over the length l of the preselected genomic DNA sequence of reference, as defined above.

In a step a), several predefined thresholds relative to said quantified predefined marker associated with the length L of said set of preselected DNA elements are chosen based on said scores, each of said predefined thresholds separating two groups representative of a different evolution of said cancer.

In a step b), for each of said predefined thresholds, the classifier computes a comparison value between two groups defined by the corresponding threshold, by using an estimation method. A selection of the thresholds corresponding to comparison values lower or higher than an arbitrary comparison value may be performed to shortlist said predefined thresholds of significance. An alternative approach may be choosing the extremum of n bins of values.

In a step c), for each combination of two thresholds of a 2-combination of predefined thresholds or shortlisted predefined thresholds, the classifier computes a comparison value between three groups defined by the two thresholds of each combination, by using an estimation method.

One extremum comparison value corresponding to one threshold from step b) may be used to split a cohort into two groups, better one extremum comparison value, corresponding to two thresholds from step c), may be used to split a cohort into three groups. In this example, as described in a step d), two reference thresholds are determined to separate three different groups representative of a different evolution of said cancer by choosing predefined thresholds that have given an extremum value of said comparison values computed at step c).

In a non-illustrated variant, only one comparison value is computed after step a), leading to the splitting of a cohort into two groups.

In the illustrated example, the Kaplan-Meier test is used as such an estimation method, said comparison value is a p-value, and said extremum value is a minimum value of said comparison values computed at step c) is considered only among combinations of thresholds for which at least 7 patients are present in each group. The estimation methods used in steps b) and c) may be the same or different. In this example, they are the same.

Steps c) and d) may be repeated with combinations of more than 2 thresholds, iPART being an iterative process. If needed, at least four groups may be generated.

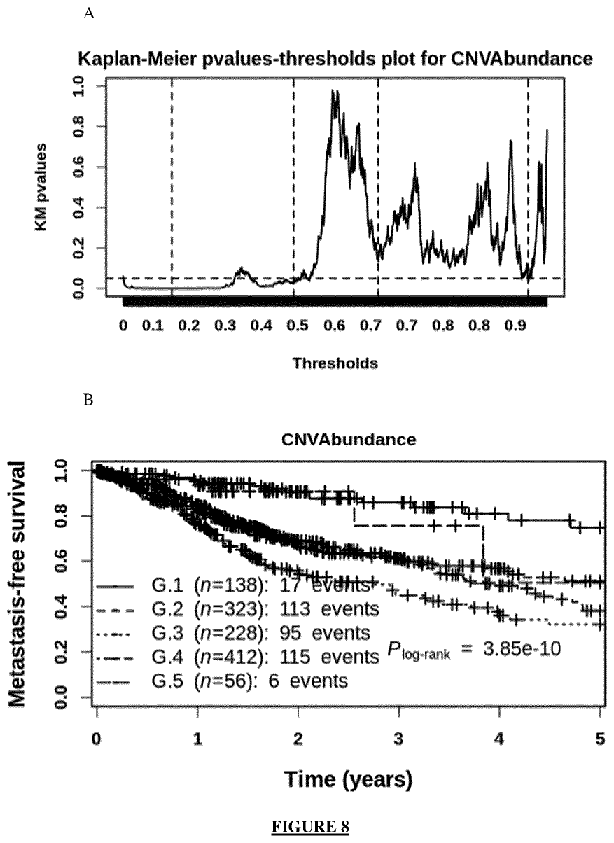

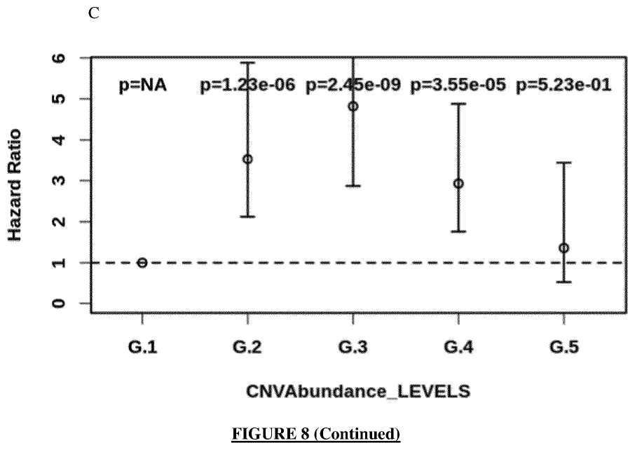

Such a method thus iterates over all possible thresholds, find thresholds that maximizes the difference between the split groups in term of the speed of clinical events occurrence in the at least two groups by minimizing the p-value of KM test, as can be seen in FIG. 8.

Part B of FIG. 1 shows steps of a method for classifying a patient having a cancer in a group representative of an evolution of said cancer according to the invention. Such a method uses said reference thresholds previously determined, a tumor genomic DNA sequence obtained by sequencing a tumor genomic DNA obtained from said patient, at least one set of preselected DNA elements, each DNA element having a genomic position, said set of DNA elements having a length L in base pairs, and a preselected genomic DNA sequence of reference having a length l in base pairs. In this example, said preselected DNA elements are selected in the group consisting of replication-associated DNA elements, transcription-independent DNA elements.

In a step a), a total quantity n of a predefined marker of DNA structure alteration is identified, with genomic positions, and quantified within said tumor genomic DNA sequence of the tumor genomic DNA from said patient, by comparing said tumor genomic DNA sequence with said preselected genomic DNA sequence of reference. Said predefined marker of DNA structure alteration is a breakpoint in the illustrated example.

In a step b), a quantity N of the predefined marker of DNA structure alteration is associated to the set of preselected DNA elements, from the total quantity n, according to the genomic positions of the DNA elements of the set of preselected DNA elements and to the genomic positions of said predefined markers of DNA structure alteration obtained at step a).

In a step c), a test score relative to a quantity N of predefined markers of DNA structure alteration associated with the length L of said set of preselected DNA elements is computed, based on a comparison between said quantity N of predefined markers of DNA structure alteration and a reference level defined by a random model relative to the total quantity n of the predefined marker of DNA structure alteration over the length l of the preselected genomic DNA sequence of reference.

In a step d), said test score is compared to said at least one reference threshold in order to classify said patient in one group among at least said two groups representative of a different evolution of said cancer.

### Example 2: Results

### Breakpoints and Hotspots/Hot Regions

Analyzing whole genome sequencing of 112 leiomyosarcomas (LMS), through our structural variants (SV) detection pipeline (see Example 1, Breakpoints identification/structural variations detection), allowed identifying 24870 breakpoint (BP) BP forming 12435 SV (each SV is formed by 2 BP). Of all BP, 67.4% (16764 BP) are implicated in intra-chromosomal SV (BPSVintra) and 32.6% (8106 BP) implicated in inter-chromosomal SV (BPSVinter). While the majority of BP occurs inside regulatory DNA elements (13377/24870=53.79%), a significant fraction affects Non-B DNA (NBD) (4902/24870=19.71%) and a minor percentage (3574/24870=14.37% of BP) arises in DNA repeats. Taken together, a total of 66.4% (16510/24870) of LMS BP are present in the DNA elements considered in this study.

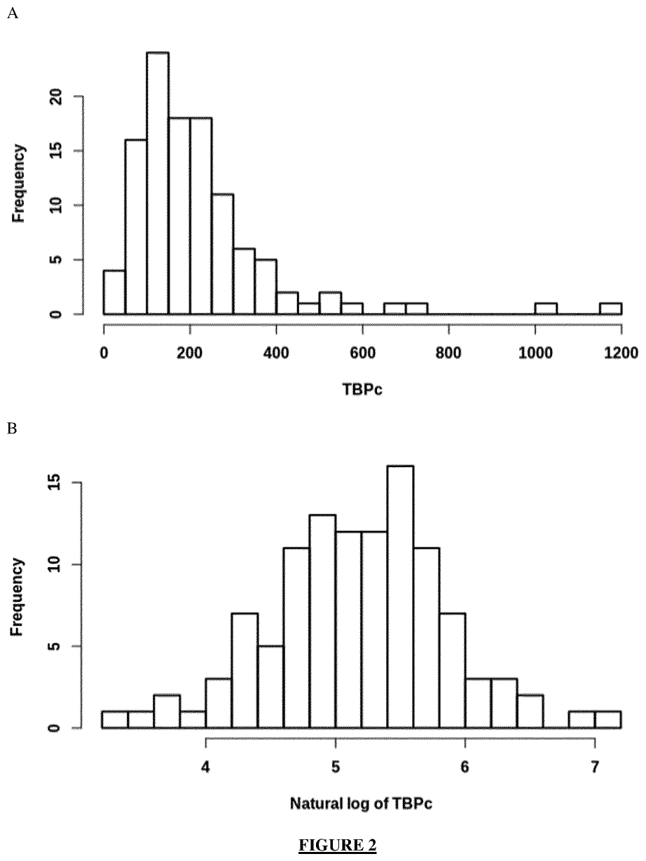

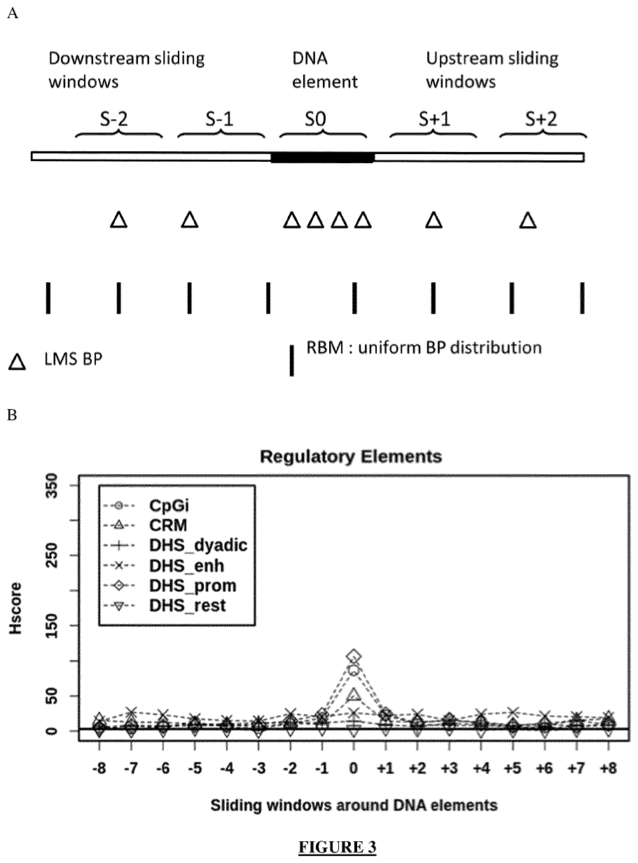

The total BP count per LMS (TBPc) is highly skewed (FIG. 2: (A) Left, mean=222.1, median=181, min=26, max=1200) and does not fit a normal distribution (Shapiro-Wilk test P=1.4×10⁻¹², FIG. 2A, left panel), but TBPc rather follows a log-normal distribution (Shapiro-Wilk test P=0.81, FIG. 2B, right panel). This is not inconsequential since log-normal distributions are common in nature and reflect forces acting independently and which interactions result in multiplicative effects (Limpert, Stahel, et Abbt, BioScience 51 (5): 341, 2001). In cancer, these forces may include genetic, environmental, physiological, immune, sub-cellular and supra-cellular constraints.

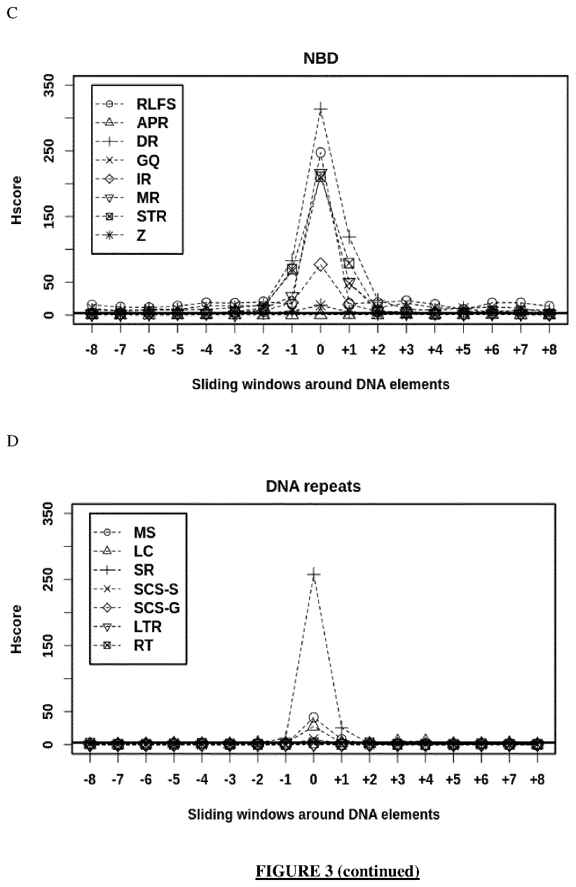

LMS genome is highly rearranged and arises the question whether DNA breakage occurs randomly or following mechanisms associated with specific regions of the genome. It was therefore asked whether there are genomic DNA structures that constitute hotspots for genome instability. To address this question, the “hotspotness” of three different types of genomic DNA structures was tested: Regulatory DNA elements, NBD, and DNA repeats. Here, BP hotspotness magnitude scale (Hscore) was introduced as a magnitude scale for the propensity of DNA elements to break more than expected by chance under RBM (FIG. 3A). Hscore is defined as −log 10 probability of having at least as observed BP, given the total number of BP, the cumulative size of DNA elements and the readable genome size (see Example 1: Materials and Methods). Hscore is very intuitive, the higher the Hscore is, the more significantly a given DNA element is altered more than expected. To determine whether a given genomic feature is a hotspot, Hscore was computed in sliding windows (see Example 1) upstream and downstream and made profile plots (FIGS. 3B, 3C and 3D; see Example 1). Two main significantly broken structures emerged: hotspots and hot regions. The Hscore threshold to retain a DNA element as a hotspot or a hot region was settled as superior or equal to 3. Then, hotspots and hotspot regions are defined as follows: 1) DNA elements significantly more broken than expected by chance (Hscore ≥3) and immediate surrounding regions are not (Hscore <3), 2) if both DNA elements and immediate surrounding regions have a Hscore ≥3, a DNA elements is considered as a hotspot if its Hscore is at least 1.5× the Hscore of both surrounding regions. A hot region is inferred if a Hscore ≥3 is observed for both DNA element and surrounding regions with Hscore of DNA element being less than 1.5 times the Hscore of surrounding regions.

### NBD and DNA Repeats are Hotspots for DNA Breakage in LMS

NBD are DNA elements that adopt non-canonical DNA structures (Gaillard et Aguilera 2016). Sequences prone to forming non-B DNA are widespread in the human genome and are associated with GI (Wang et Vasquez, DNA Repair 19 (juillet): 143-51, 2014). The formation of non-B DNA conformations requires unwinding of the DNA sequence, as occurs during replication, transcription (Gaillard et Aguilera 2016). NBD comprise A-phased repeats (APR), direct repeats (DR), G-quadruplex (GQ), inverted repeats (IR), mirror repeats (MR), short tandem repeats (STR), Z DNA (Z) and R-loops forming sequence (RLFS). We found that all NBD but APR are hotspots (FIG. 3C), notably, GQ which are very highly significantly broken so that Hscore tends to infinity (P=0).

DNA repeats instability is also a major threat to genome integrity. DNA repeats included in this study comprise microsatellite (MS), simple repeats (SR), Low complexity (LC), self-chains segments (SCS) which were classified into self-aligned (SCS-S) and self-chains segments gaped (SCS-G), Long Terminal Repeats (LTR), Retro Transposon (RT). While SR, MS, LC are hotspots, viral origin repeats LTR and RT are not (FIG. 3D). Furthermore, while SCS-S were hotspots, SCS-G were not (FIG. 3D).

### Genes Promoters are Hotspots, Gene Enhancers are Hot Regions

The promoters of transcriptionally active genes have repeatedly been shown to recurrently harbor double-strand breaks (DSB) (Marnef, Cohen, et Legube, Journal of Molecular Biology 429 (9): 1277-88, 2017). Also, DNase I hypersensitive site (DHS) as well as active chromatin marks have been shown to colocalize with DSB (Mourad et al., Genome Biology 19 (1): 34 2018). It was therefore sought to quantify the contribution of regulatory DNA elements to LMS Genome Instability (GI). Regulatory DNA elements used in this study comprise CpG islands (CpGi), CRM, DHS of promoter type (DHS_prom), of enhancer type (DHS_enh), of dyadic type (both enhancer and promoter signatures) (DHS_dyadic), and of other types (DHS_rest) (Roadmap Epigenomics Consortium et al., Nature 518 (7539): 317-30, 2015). It was found that promoters associated DNA elements (i.e. DHS_prom and CpGi) are hotspots for DNA breakage (FIG. 3B) while DNA elements associated with enhancer activity (DHS_Enh) are hot regions (FIG. 3B). Furthermore, promoters associated regulatory elements (CpGi, DHS_prom) break more significantly than enhancers (DHS_enh) which break more significantly than Dyadic regulatory elements (DHS_dyadic) and rest of DHS (DHS_rest) (FIG. 3B). Interestingly, CRM, which is a merge of overlapping regulatory regions (promoters, enhancers, dyadic; Chèneby et al., Nucleic Acids Research 46 (D1): D267-75, 2018) is a hotspot and has an intermediate Hscore further stressing the robustness of the BP hotspotness magnitude scale (FIG. 3B). Finally, DHS which are not labeled to have any gene regulatory function (DNH_rest) are indistinguishable from random. Taken together, these results show that promoters break more significantly than enhancers which break more significantly than dyadic regions. Furthermore, open chromatin structures (DHS) with attributable gene's regulatory function like enhancers and promoters breaks significantly than random while DHS with no attributable gene's regulatory function are randomly broken. Therefore, DHS do note break merely to the openness of their chromatin but rather due to their genic context. The analysis was refined by splitting each DNA elements in two categories: located inside or outside genes.

Regulatory Elements are Almost Exclusively “Hot” Inside Genes and not Outside them

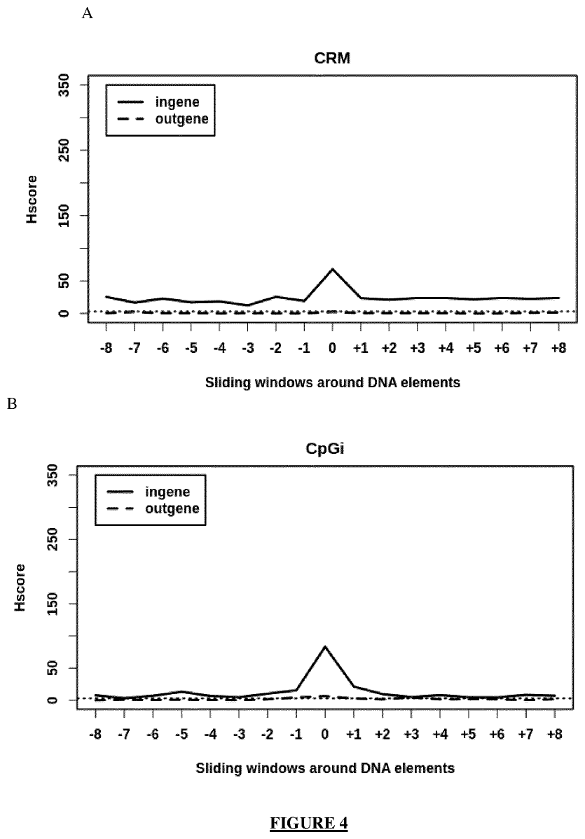

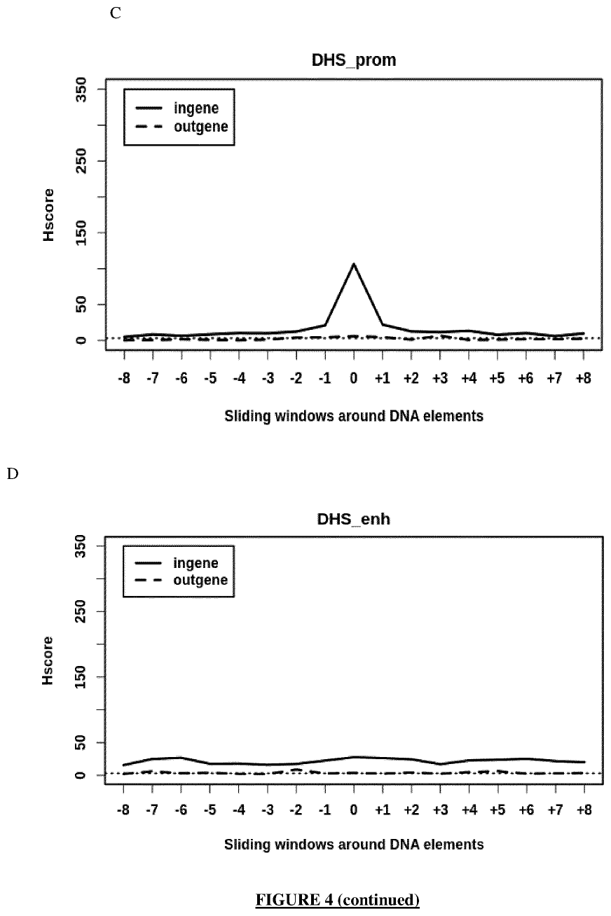

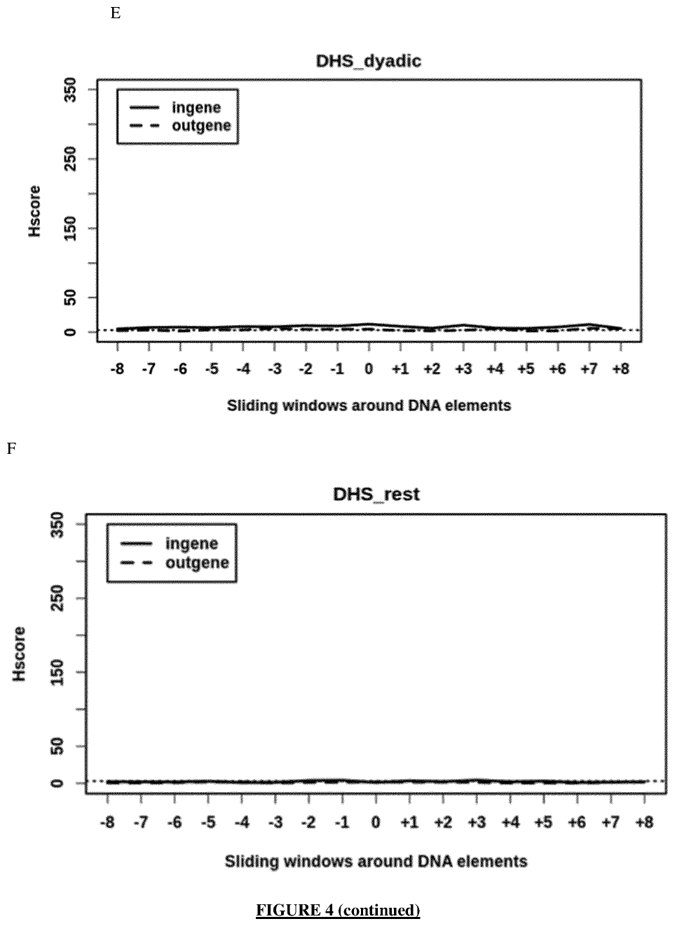

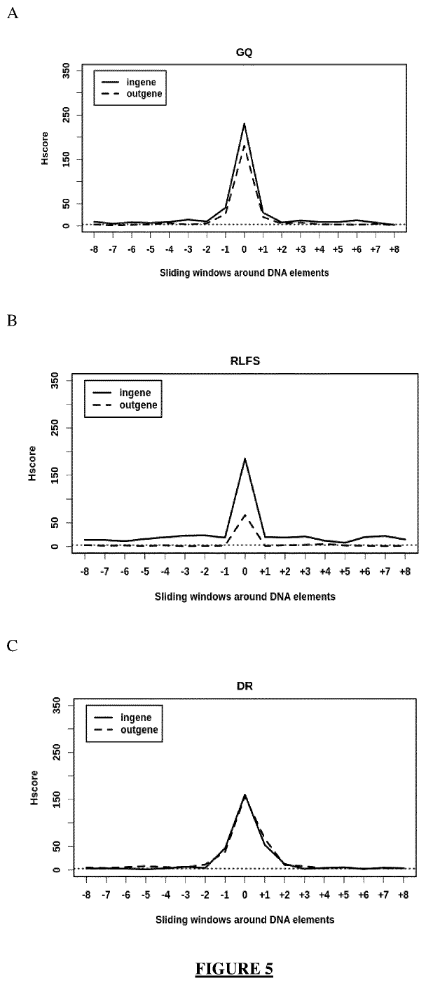

In order to address the relationship of regulatory DNA elements to their genic context they were split into those located inside genes from transcription start site (TSS) to transcription termination site (TTS) (including DNA elements with at least 1 bp overlapping with genes) and those located outside genes. Hscore were computed for each group in sliding windows. It was found that all regulatory elements (FIG. 4A to F) are more significantly broken when they are located inside genes than outside, independently of their structure (sequence type CpGi vs DNA-protein complexes) or function (promoter type vs enhancer type). CRM are exclusively broken more frequently than by chance when located inside genes with Hscore of 67.82 inside genes and less than 3 outside (FIG. 4A, and table 1). Although CpGi located outside genes are slightly significantly broken (Hscore=6.41), this significance is much higher when they are located inside the genes (Hscore=83.30) (FIG. 4B) and ratio of Hscores inside/outside genes of 12.99 (RHscore i/o) (see table 1). Similarly, all types of DHS regulatory elements are broken more significantly inside genes than outside (FIGS. 4C, D, E and F). These results suggest that the transcriptional and/or epigenetic context of genes strongly modulate the ability of DNA regulatory elements to harbor DNA breakpoints. Since regulatory elements, when active, have an open DNA structure both inside and outside genes, we exclude the possibility that their propensity to harbor DNA BP is merely due to the openness of the chromatin.

### NBD and DNA Repeats are Hotspots Both Inside and Outside Genes

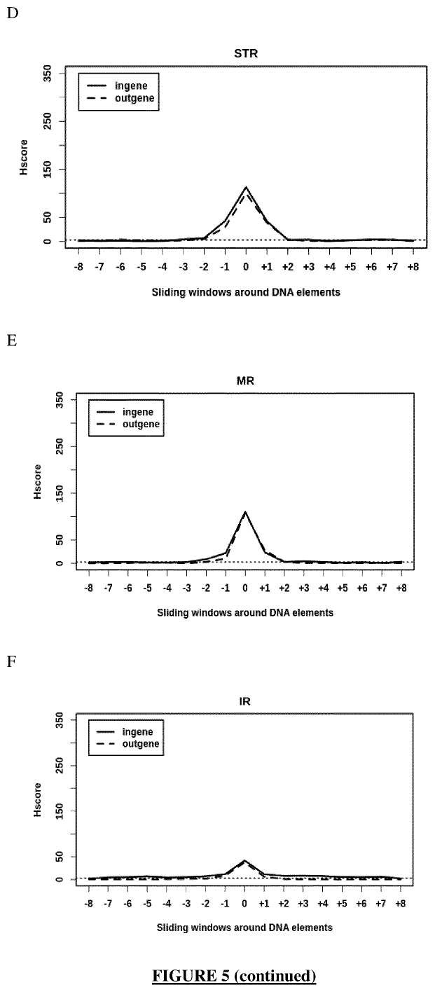

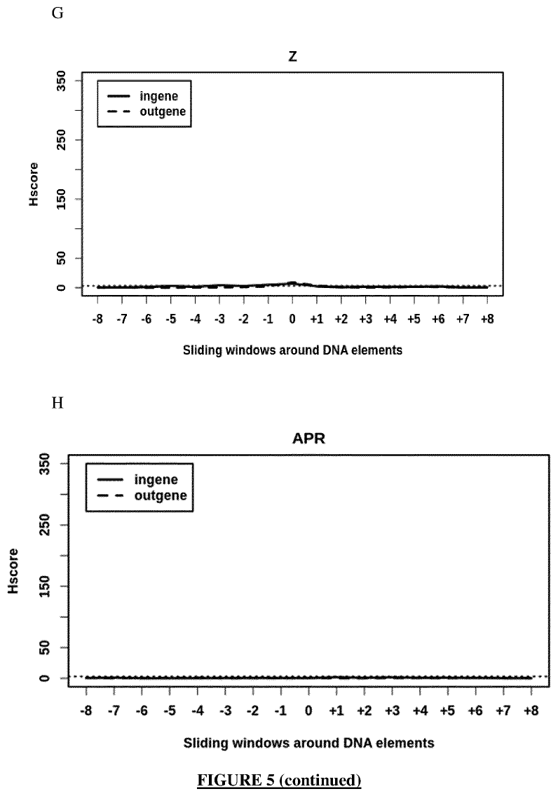

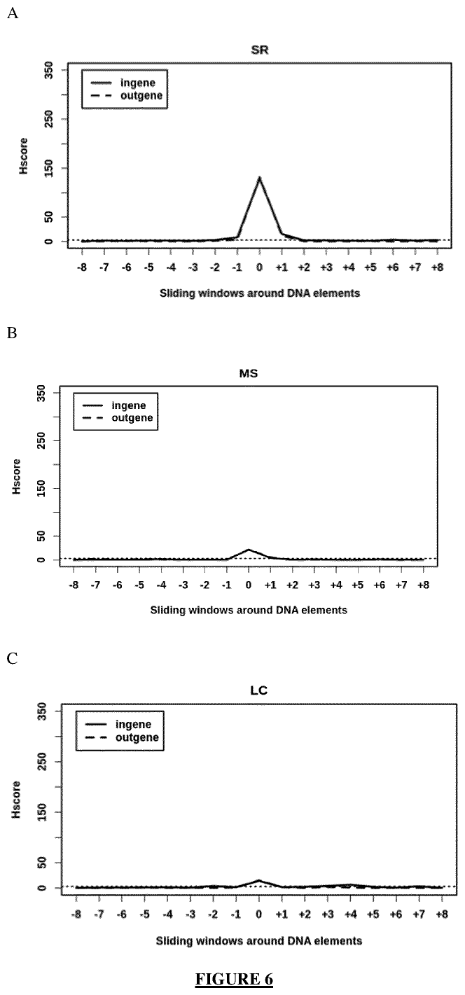

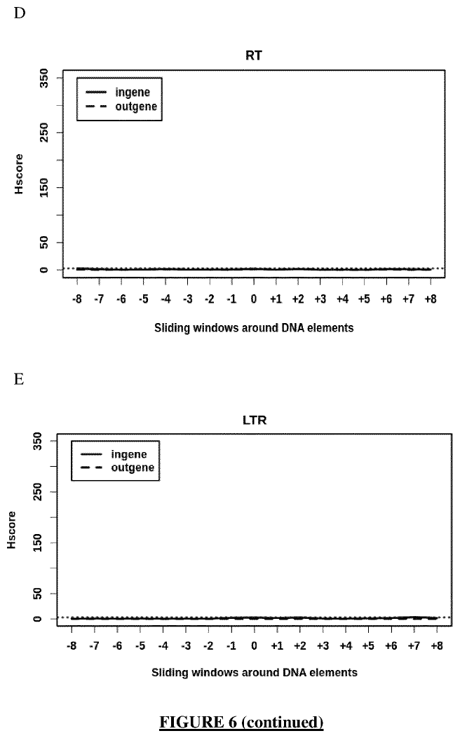

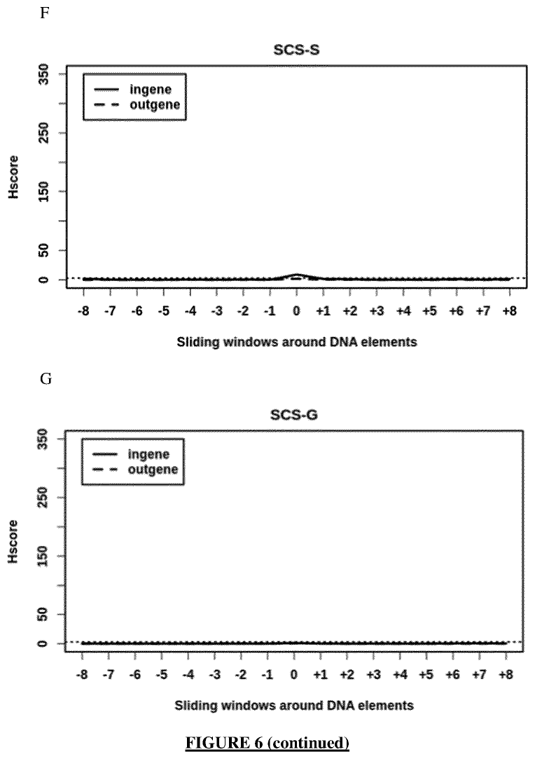

NBD and DNA repeats were split into those located inside genes and those located outside them (FIG. 5). MR, IR, DR, and STR are equivalently significantly broken inside and outside genes (i/o) (Ratio Hscore i/o 1.02, 1.012, 1.02, 1.13 respectively). While RLFS are highly significantly enriched in BP outside genes (Hscore=66.24), they are even more significantly broken inside them (FIG. 5B) (Hscore=185.41, RHscore i/o=2.8) (see table 1). GQ are also highly significantly enriched in BP outside genes (Hscore=180.53) and present a tendency to be more broken inside them (Hscore=230.17, RHscore i/o=1.27) (FIG. 5A). Z DNA seems to have a tendency to be more frequently broken outside genes than inside them (RHscore i/o=0.73) (FIG. 5G). Hscore profiles of DNA repeats located inside genes and outside genes are almost identical except for SCS-S, which are significantly more broken than random inside genes (Hscore=8.94) but not outside them (Hscore=1.18<3) while SCR are indistinguishable from random (FIGS. 6F and G). Together these results suggest that, except for SCS-S, GQ and RLFS, NBD and DNA repeats are insensitive to gene context and may cause genomic instability through a mechanism predominantly independent from transcription and which is most probably replication dependent. It is noteworthy to say that GQ and RLFS have properties of both regulatory elements by being sensitive to their genic context and as DNA repeats and NBD, by being breakable when located outside of genes as well.

### Not all LMS has BP Distributed as Hotspots.

The previous results obtained on the whole cohort were global and did not describe what happens for each single patient. To evaluate DNA breakage mechanisms acting in each patient, Hscores were computed for regulatory elements, NBD and DNA repeats, in each LMS tumor sample and made a hierarchical clustering on LMS patients based on these Hscores (Example 1). It was observed that not all LMS patients have BP distributed as hotspots and that there is a gradient of hotspotness. Furthermore, each LMS sample has its specific profile: while some patients have no hotspots detected, some patients have BP hotspots mainly in the regulatory regions, others mainly in NBD and DNA repeats and other in combination of regulatory elements and NBD and DNA repeats. Interestingly, metastatic and non-metastatic patients tend to be not evenly distributed over the gradient of hotspotness, the left half of the heat map having less metastatic events than the opposite side. The question of the relation between BP hotspotness and patient prognosis was addressed with different approaches in the next sections.

The LMS Cohort can be Stratified into Clinically Relevant Groups.

Because first, regulatory elements, NBD and DNA repeats have been thoroughly documented to impede transcription and replication in both transcription-associated (Gaillard et Aguilera 2016) and replication-associated (Gaillard, García-Muse, et Aguilera, Nature Reviews Cancer 15 (5): 276-89, 2015) manners, it was hypothesized that there is a link between transcription and replication-associated DNA-breakage mechanisms and metastatic clinical outcome of LMSs. To test this hypothesis, it was sought to quantify the overall transcription dependent and independent DNA-breakage/genome instability. It was taken advantage of the BP hotspotness magnitude scale and derived genomic indexes for both transcription-associated and transcription-associated DNA-breakage and genome instability. As DR, STR, MR, IR, Z DNA, SR, MS, and LC are BP enriched independently of their position inside or outside the genes, therefore those elements were considered as replication-associated chromosomal instability elements (RACINe). Conversely, RLFS, GQ, CpGi, CRM, SCS-S and DHS were considered as transcription-associated genomic instability elements (TRACe) because they are more frequently broken than chance inside genes and not outside them. By consolidating all of the TRACe and RACINe into one functional group each and computing Hscores (Example 1), TRAC index (TRACi (or iTRAC)) and RACIN index (RACINi (or iRACIN)) were, respectively, derived. TRACi (or iTRAC) and RACINi (or iRACIN) allow the quantification of the overall contribution of RACINe and TRACe to DNA-breakage and therefore to genomic instability (GI) in each LMS patient. To address the relationship between TRACi (or iTRAC), RACINi (or iRACIN) and metastatic clinical outcome, a method called iPART for Iterative multi-thresholds PARTionning (iPART) was developed (see Example 1).

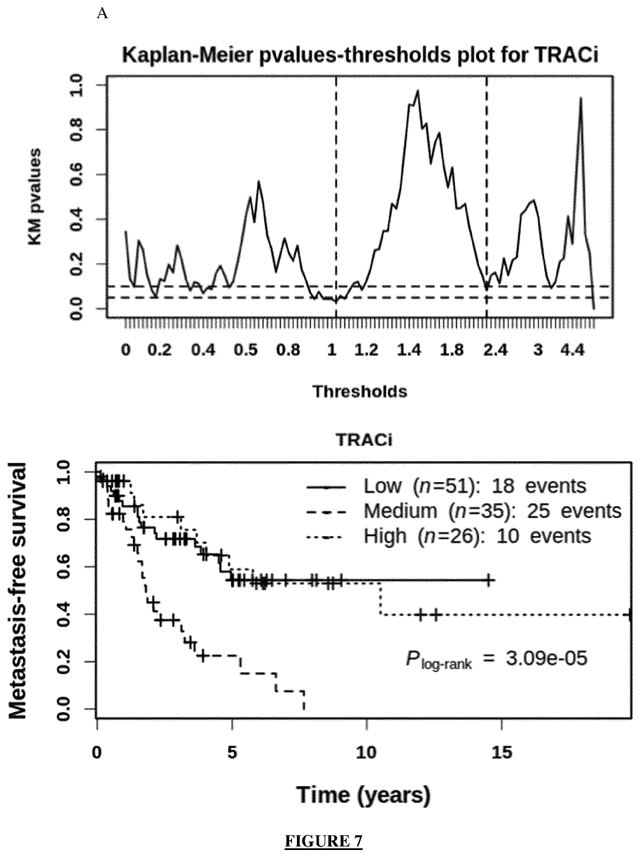

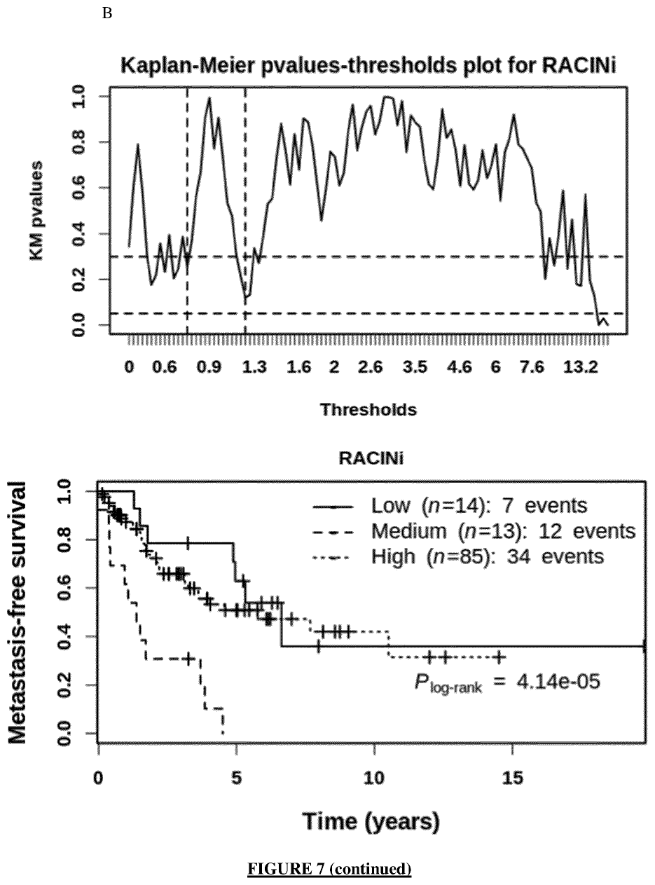

First, iPART, as described above, was used to look for a threshold for TRACi (or iTRAC) and RACINi (or iRACIN) that splits LMS cohort into two groups with a maximum MFS (Metastasis Free Survival) difference. FIGS. 7A left and 7B left panels show that TRACi (or iTRAC) has several thresholds giving p-values less than 0.05 (black asterisk) while RACINi (or iRACIN) has only few at very high values only. It was also noticed the presence of several local minima suggesting that there will be more than one “natural frontier” in the data. To test this hypothesis, iPART was applied using all combinations of two thresholds from all local minima below 0.10 for TRACi (or iTRAC) and 0.3 for RACINi (or iRACIN) (horizontal dashed line) (see Example 1). Accordingly, both TRACi (or iTRAC) and RACINi (or iRACIN), very highly significantly stratified the LMS cohort (P=3.08×10⁻⁵ and P=4.13×10⁻⁵, respectively) into three groups showing statistically significant difference in the risk of metastatic events occurrence (FIGS. 7A and 7B bottom panels). Strikingly, it is the groups of LMSs with medium TRACi (or iTRAC) and medium RACINi (or iRACIN) which have the most unfavorable outcome (FIGS. 7A and 7B bottom panels).

Sheer BP Counts Far Less Significantly Stratified Metastasis Risk than TRACi (or iTRAC) and RACINi (or iRACIN) in LMS Cohort

iPART was applied on TBPc, number of BP implicated in intra-20 chromosomal SV (nBPSVintra), number of BP implicated in inter-chromosomal SV (nBPSVinter). It was found that although TBPc, nBPSVintra and nBPSVinter were slightly significant (see FIG. 11), they do not compare by any measure to the level of significance achieved by TRACi (or iTRAC) and RACINi (or iRACIN).

### Mixed Transcription- and Replication-Associated Genomic Instability Classifier (MAGIC)

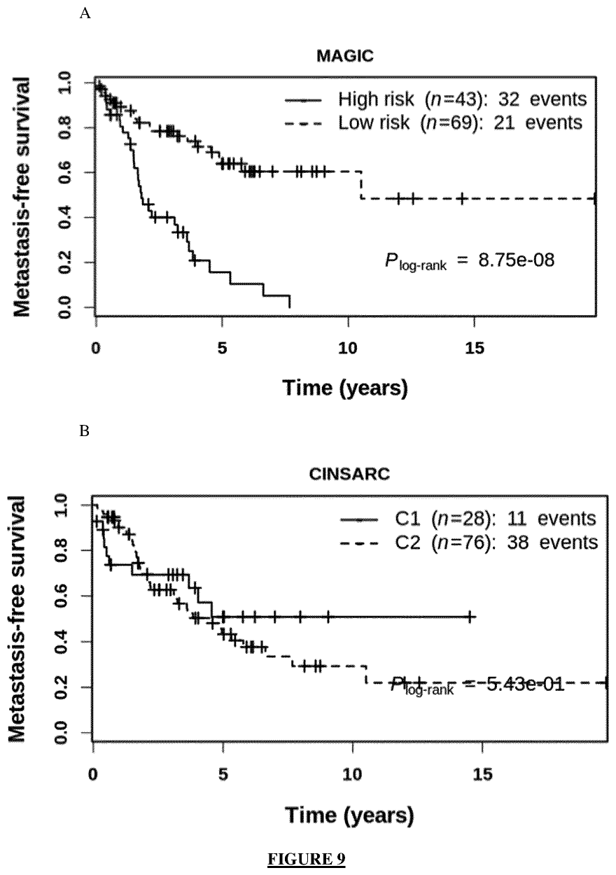

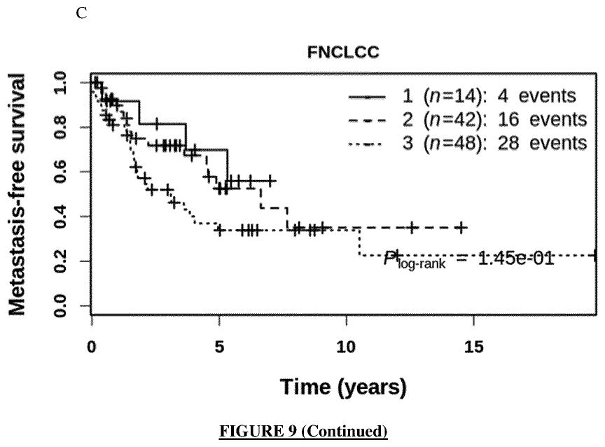

In order to translate these results to a clinically relevant stratification tool, it was sought to integrate both TRACi (or iTRAC) and RACINi (or iRACIN) into one classifier: MAGIC for ‘Mixed transcription- and replication-Associated Genomic Instability Classifier’. Given that both indexes have high and comparable statistical significance in stratifying LMS, it was merged in high metastasis risk group (MAGIC High risk) any patient which is classified medium by at least one of the TRACi (or iTRAC) and RACINi (or iRACIN) and in low risk the rest of patients (MAGIC Low risk). Using MAGIC, it was possible to achieve a very high level of significance in stratifying LMS samples (P=8.75×10⁻⁸; FIG. 9A), with a median MFS for MAGIC High risk group of 1.8 (CI=[1.52, 3.61]) which is 5 times less than for MAGIC Low risk group (10.5, CI=[5.78, NA]).

iPART Significantly Stratified a Pan-Cancer Cohort of Twelve Cancer Types into Clinically Relevant Groups

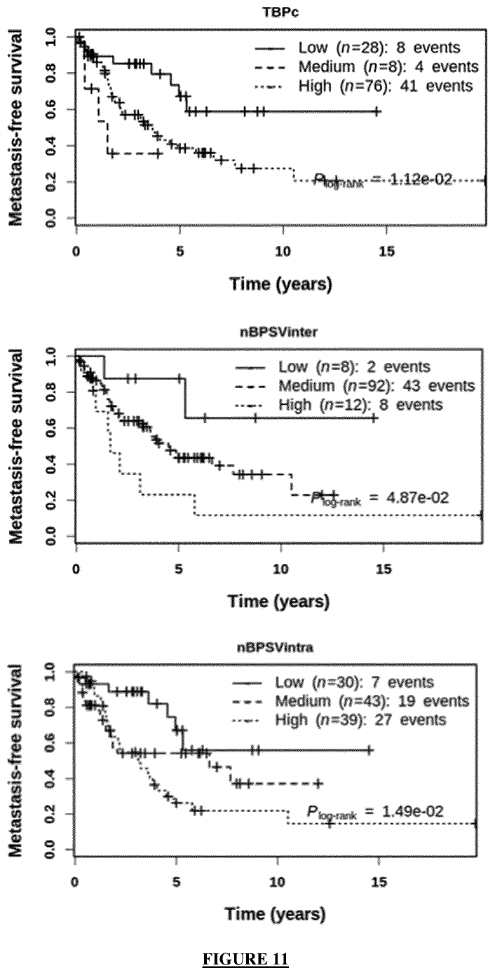

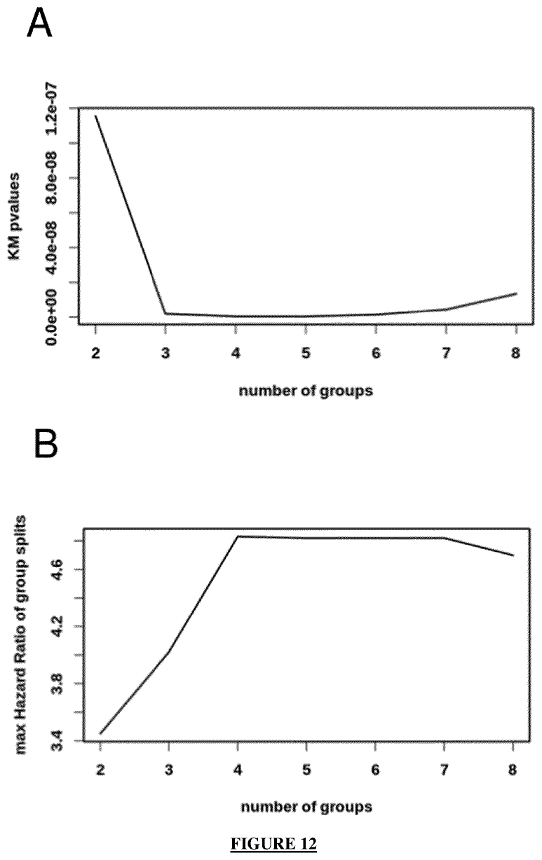

Intermediary level of GI resulting in bad clinical outcome compared to low and high levels has also been reported in a Pan-Cancer study of 12 cancer types (TCGA cohort) (Andor et al. 2016). The authors used CNV abundance as a measure of GI and found that CNV affecting between 25% and 75% of a tumor's Meta-genome was predictive of poor survival. It was thus hypothesized that what was observed in LMS would be a general mechanism associated with tumor aggressiveness. Actually, this question was intended to be tackled in a reasonable time scale, computational and resource-wise manner, applying the methods as disclosed herein directly on CNV abundance from “Andor et al 2016” as a proxy for TRACi (or iTRAC)/RACINi (or iRACIN). The arbitrary and commonly used method of splitting the data into quartiles based on thresholds of 25%, 50%, and 75% to segments the cohort into four groups based on CNV abundance in the tumors meta-genome has been used. Using iPART, the Andor's cohort was split into 2, 3, 4, 5, 6, 7 and 8 groups and it was evaluated each time the relevance of that data segmentation on the risk of mortality using Log-rank test and hazard-ratio (FIGS. 8 and 12). The best data segmentation corresponds to 5 groups split with log-rank test P=6.9×10⁻¹⁰ and maximal HR=4.7(p=3.42×10⁻⁹, FIG. 12). In other hand, data segmentation applied by Andor et al. has a Log-rank test P=5×10⁻⁶ (FIG. 5b of results section of Andor et al, see FIG. 13) and a maximum HR no more that 1.9 with P<0.005 (FIG. 5c of Andor et al). Thus, similarly to what Andor et al. showed, it is the intermediate group (G.3) which is associated with the worst overall survival and presents the highest HR (HR=4.75, p=3.42e−9), as shown in FIG. 8. These results strengthening the robustness of the new approach, the MAGIC prognostic value was then compared to those of established sarcoma prognostic tools.

### MAGIC Outperforms Histologic FNCLCC and Molecular CINSARC Gradings

Histological FNCLCC grading system, predicting patient evolution, is the current standard in sarcomas (Coindre et al., Cancer 91 (10): 1914-26 2001; Guillou et al., Journal of Clinical Oncology 15 (1): 350-62, 1997). CINSARC is the best ever tested molecular signature in sarcomas, challenging this histological gold standard and currently under clinical investigation for stratification (Chibon et al., Nature Medicine 16 (7): 781-87, 2010). Both approaches, taken individually, did not significantly split the LMS cohort in groups with different metastatic evolution (FIG. 9). This result precludes any introduction of one if this grading system in a multivariate analysis and it was thus concluded that MAGIC strongly outperforms both FNCLCC grading system and CINSARC in LMS metastasis risk stratification.

TRACi (or iTRAC) Significantly Stratified Chemotherapeutic Response in LMS Cohort

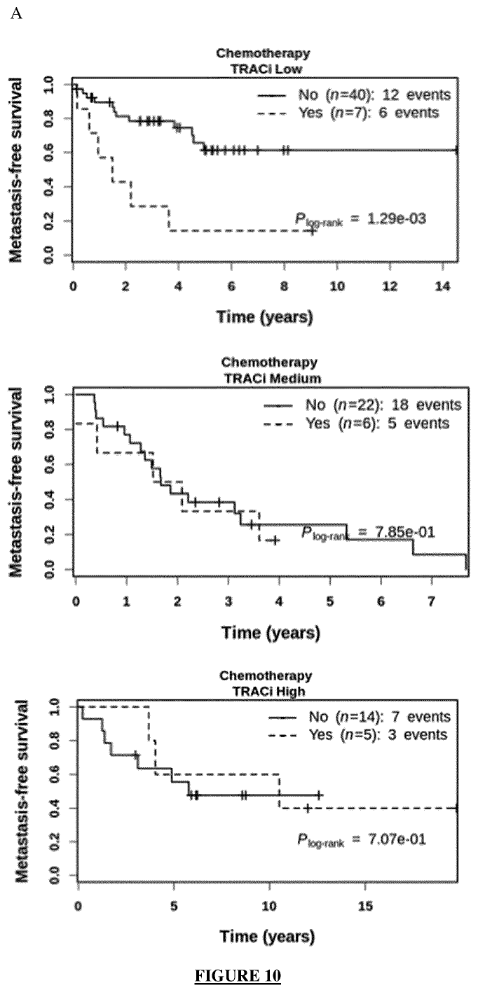

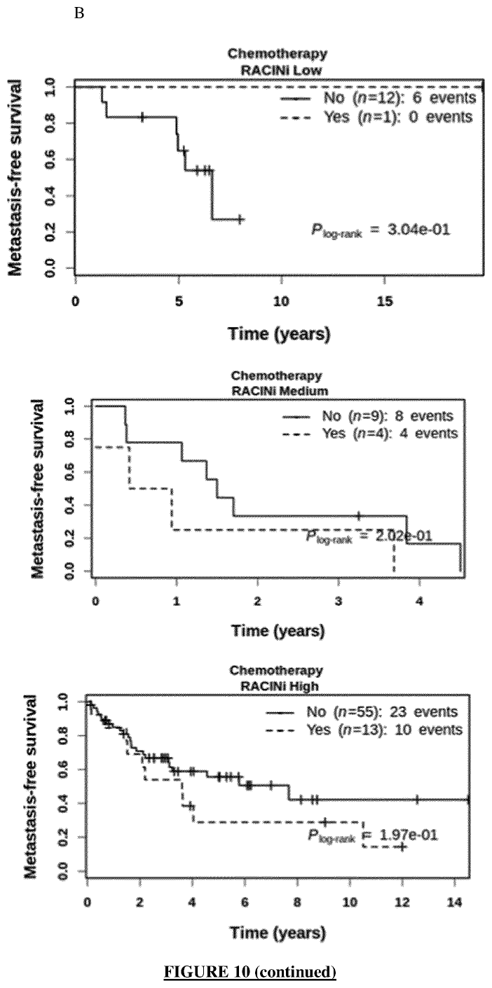

Chemotherapy is a still controverted therapeutic approach in LMS since non-clinical trial has ever demonstrated its benefit. The hypothesis explaining this setting is that candidates, i.e. patients with a poor prognosis and responding to chemotherapy, are still not efficiently selected. This was addressed, and among the 112 LMS of the studied cohort, 18 underwent chemotherapy (14 adjuvant, 1 neoadjuvant and 3 palliative chemotherapy), 18 patients were not annotated (NA) and 76 patients we not treated with chemotherapy. Each of the Low, Medium, and High groups of TRACi (or iTRAC) and RACINi (or iRACIN) were then split according to treatment status, i.e. Yes (18 pts) or No (76 pts). Interestingly, while in Low risk TRACi (or iTRAC) group, patients receiving chemotherapy have a poorer prognosis (HR=4.47, CI=[1.65, 12.08],p=0.0032), in Medium and High risk TRACi (or iTRAC) groups no therapeutic benefices were found (FIG. 10). In the High risk TRACi (or iTRAC) group, there is a tendency that chemotherapeutic intervention could be beneficial.

### Example 3: Discussion

The invention provides new tools, measures, and insights to tackle the question of the Leiomyosarcoma clinical outcome and its relation to genome rearrangement. Deciphering instability mechanisms, a tool called MAGIC was established, accounting for transcriptionally related or independent genomic instability, which has demonstrated prognostic value outperforming both actual histologic gold standard and molecular challenger grading systems. Moreover, this approach is potentially applicable on Pan-Cancer as emphasized from the significant prognosis that was established in twelve different cancer types using iPART as disclosed herein and CNV abundance as a proxy for TRACi (or iTRAC)/RACINi (or iRACIN).

### Application of Hscore on TRACe and RACINe as a Holistic Approach to Measuring GI

Recent studies have used different GI scores and indexes to predict clinical outcome and to define homologous recombination (HR) deficient samples (Zhang, Yuan, et Hao, Gautam Chaudhuri. PLoS ONE 9 (12): e113169, 2014; Birkbak et al., Cancer Discovery 2 (4): 366-75, 2012; Abkevich et al., British Journal of Cancer 107 (10): 1776-82 2012; Popova et al., Cancer Research 72 (21): 5454-62, 2012; Stefansson et al., Breast Cancer Research 11 (4): R47 2009; Baumbusch et al., Alexander James Roy Bishop. PLoS ONE 8 (1): e54356, 2013; Mirza et al., New England Journal of Medicine 375 (22): 2154-64, 2016). However, most of these studies were merely based on the per patient counts of BRCA1/2 mutations, genome SNPs, loss of heterozygosity (LOH), or specific structural variations (CNV, Telomeric Allelic Imbalance (TAI), etc.). These studies are based on the assumption that an a priori selection of chosen genomic alterations, could capture the dynamics of GI in a tumor that is complex, heterogeneous, and subjected to Darwinian selection. Here it was presented a new measure of genomic instability which is rather based on the idea that GI is mainly due to the disruption of the steady state equilibrium between continuous DNA damage and matched level of high fidelity repair maintaining genome integrity (Tubbs et Nussenzweig, Cell 168 (4): 644-56, 2017).

The present invention introduces Hscore which is a BP hotspotness magnitude scale, measuring of the propensity of a given functional and/or structural genomic DNA element to harbor BP more than expected by chance. Application of Hscore on TRACe and RACINe is a holistic approach to measure GI based on measuring the steady-state equilibrium between DNA damage and repair by quantifying the residual DNA BP remaining after unsuccessful repair irrespective of SV type and quantifying the relative contribution of two main contributor to GI: TRAC and RACIN. Hscore is a measure on all tumor BPs without any biological-rational selection bias toward SV types. Values are comparable between different DNA elements inside a patient and between patients. The higher the Hscore is the more unlikely the observed BP are due to random events and therefore are more likely due to structural and or functional properties of those DNA elements.

### MAGIC a New Standard for LMS Grading

LMS prognostication is still challenging and mandatory to evaluate which therapeutic intervention can benefit to which patient. Integrating that some patients have a poor prognosis associated to a transcription stress and some to a replication stress, a simple patient stratification tool called MAGIC was thus derived. While in LMS cohort, both histologic FNCLCC and molecular CINSARC gradings are not suitable predictive methods, the MAGIC classifier presented herein reached a high level of significance, with the high risk group having median MFS=1.8 years, five time 5 lesser than low risk group (MFS=10.5 years). MAGIC is proposed to clinicians as tools to spot patients with high risk of metastasis for more regular follow-up.

Prognostic Relevance of TRACi (or iTRAC) and RACINi (or iRACIN) for Metastasis Risk Stratification of LMS

Strikingly, and contrary to what anyone would expect, the risk of metastasis follows what could be qualified as a lambda (A) shape with the increase of TRACi (or iTRAC) and RACINi (or iRACIN). Actually, Low and High GI levels correspond to a lower metastatic risk and medium level to a higher metastatic risk. Interestingly, similar results were previously reported in a Pan-Cancer analysis of 12 cancer types (Andor et al 2016) with regards to the abundance of copy number variations (CNV) but with far less statistical significance, highlighting the added value of the quantification of transcription- and replication-associated GI and iPART algorithm over simple SV counting. These results strongly suggest that the approach presented herein would work on different cancer types and by extension to other highly remodeled cancer genomes.

Predictive Relevance of TRACi (or iTRAC) and RACINi (or iRACIN) for Chemotherapeutic Response in LMS

The overall contribution of curative and adjuvant cytotoxic chemotherapy to 5-year survival in adults was estimated to be 2.3% in Australia and 2.1% in the USA (Morgan, Ward, et Barton, Clinical Oncology 16 (8): 549-60, 2004). Furthermore, it has been estimated that any particular class of cancer drugs is ineffective in 75% of patients (Personalized Medicine Coalition, The personalized medicine report—Opportunity, challenges, and the future, 2017). Thus, predicting which patients are eligible to which treatment and those who are not is crucial in precision medicine. It is shown here that chemotherapeutic treatment for patients with Low TRACi (or iTRAC) is detrimental for their MFS (Metastasis Free Survival) and therefore should be prohibited. Chemotherapeutic treatment is with no clinical benefices for medium TRAC. They should be proposed another therapeutic strategy. No final conclusion could be drawn for chemotherapeutic response in High TRACi (or iTRAC) because of the low statistical power of log-rank test due to the low number of patients in High TRACi (or iTRAC) which underwent chemotherapy. Nevertheless, High TRACi (or iTRAC) patients are probably to benefit from chemotherapy. RACINi (or iRACIN), in another hand, shows no predictive relevance in the stratification of chemotherapeutic response. Nevertheless, it is expected RACINi (or iRACIN) to be relevant in the stratification of targeted therapies based on targeting replication and replication-associated repair

The Relevance TRACi (or iTRAC)/RACINi (or iRACIN) for Precision Medicine and Oncology

GI can have either oncogenic or tumor-suppressive effects depending on its dynamics. Indeed, the efficacy of some cancer treatments that induce DNA breakage, such as paclitaxel and radiation therapy, is improved in cells with a higher basal rate of GI (Bakhoum et al., Nature Communications 6 (1): 5990, 2015; Janssen, Kops, et Medema, Proceedings of the National Academy of Sciences 106 (45): 19108-13, 2009; Zasadil et al., Science Translational Medicine 6 (229): 229ra43-229ra43, 2014). Conversely, ongoing GI accelerates the development of anticancer drug resistance often leading to treatment failure and tumor relapse, which limit the effectiveness of most current therapies (Sansregret et al., Nature Reviews Clinical Oncology 15 (3): 139-50, 2018). Also, immune evasion and immunotherapy failure has also been linked to tumor aneuploidy (Davoli et al., Science 355 (6322): eaaf8399, 2017). Thus, GI may offer a mechanism of escape following treatment by chemotherapeutic drugs if administered for tumors which are far below the threshold of GI that induce tumor cells death and would have beneficial effect if administered in tumors just below that threshold. Similarly, ongoing GI might constitute an immunogenic trigger or may also give a mechanism of escape of the immune system leading to failure of immunotherapy. iPART/TRACi (or iTRAC)/RACINi (or iRACIN)/MAGIC are proposed as toolset to tackle the question of the relation of GI to therapeutic intervention in an informed way on the genomic processes undermining genomic integrity. Based on what previously said, it can be appreciated that Medium TRACi (or iTRAC) would have better immunotherapeutic responses while Low TRACi (or iTRAC) would not be immunogenic and High TRACi (or iTRAC) would develop escape mechanisms

### Targeted Therapy as a Therapeutic Strategy in LMS.

Our understanding of GI may serve both prognosis and orienting the choice of therapeutic agent. Indeed, PARP1 inhibitors have an established therapeutic effect in 1-5% of women with breast cancer who have inherited mutations in BRCA1 or 2 (Ahmad, Ahmed, et Venkitaraman, Clinical Oncology 30 (12): 751-55 2018). Furthermore, it has been proposed that most LMS tumors display hallmarks of “BRCAness” including alterations in homologous recombination DNA repair genes, enrichment of specific mutational signatures, and cultured LMS cells are sensitive towards olaparib and cisplatin (Chudasama et al., Nature Communications 9 (1): 144, 2018). Thus, PARP1 inhibitor would be therapeutic to consider in LMS. The use of TRACi (or iTRAC)/RACINi (or iRACIN) in clinical settings would allow for selection for LMS patients with significant therapeutic response to PARP1 inhibitors.

Given the far-reaching consequences of GI for treatment success, therapeutic choices and clinical care, an accurate measure of GI and its dynamics is paramount of precision medicine. The development of robust biomarkers enabling GI dynamics to be captured is crucial if one is to leverage the potential of GI for patient stratification purposes and for exploiting this feature for orienting therapeutic choices. Deriving minimally invasive approaches that enable clinicians to assess whether or not ongoing GI is taking place within a given tumor sample might be crucial for efficient exploitation of GI in the clinical setting. Thus, measuring TRACi (or iTRAC)/RACINi (or iRACIN) in circulating tumor DNA (ctDNA) or circulating tumor cells (CTC) would be an attractive approach to be explored in the future.

### Example 4: Use of the Exome to Predict Therapy Responsiveness in Cancer Patients

### Materials & Methods

### Samples

The selection of patients and of the protocol for obtaining and preparing the sample are as in Example 1.

### DNA Extraction

Genomic DNA was obtained as indicated in Example 1.

### Exome Sequencing and Analysis

A “simulated ExomeSeq” was used: Whole Genome Sequence data analyzed by a mutation caller GATK 4 (Van der Auwera G A & O'Connor B D. (2020)), then mutations only present in the exomes were retained. For indels, was retained only indels which are entirely inside a single exon.

Measures of DNA Events in the TRAC Index/RACIN Index (iTRACexome/iRACINexome)

The detection of the somatic short mutations and identification of breakpoints were carried in the DNA elements of the simulated sequenced exome as indicated in Example 1, except that the mutation caller, GATK 4, was used instead of our home made algorithm.

The sets of DNA elements of the two indexes iTRACexome and iRACINexome were comprised of, respectively, of direct repeats (DR), short tandem repeats (STR), mirror repeats (MR), inverted repeats (IR), Z DNA, simple repeats (SR), microsatellites (MS), low complexity DNA (LC), for iRACINexome and of R-loops forming sequences (RLFS), G-quadruplexes (GQ), and CpG islands (CpGi), for iTRACexome. Further, the two sets of DNA elements were merged in one set of DNA elements: iTRACINexome (transcription- and replication-associated chromosomal instability elements), comprising direct repeats (DR), short tandem repeats (STR), mirror repeats (MR), inverted repeats (IR), Z DNA, simple repeats (SR), microsatellites (MS), low complexities (LC), R-loops forming sequences (RLFS), G-quadruplexes (GQ), and CpG islands (CpGi).

### Selection of Hscore for Patients Stratification

Hscore was computed as indicated in Example 1 but with exome as reference genome.

Patients classified with a Hscore below 3 (assigned to Group 1) were considered as having a tumor DNA not significantly broken in the sets of iTRACexome and iRACINexome DNA elements. There is no accumulation of DNA Break points in hotspots (iTRACexome and iRACINexome DNA elements) and BP are randomly distributed in the exome/TRAC and RACIN elements. Patients classified with a Hscore above 3 (assigned to Group 2) were considered as having a tumor DNA significantly broken in the sets of iTRACexome and iRACINexome DNA elements.

### Measure of the Tumor Burden Mutations (TMB)

TMB is computed as total number of identified breakpoints in the reference exome over its size multiplied by 1000000.

iTRACexome, iRACINexome and iTRACINexome

TRACexome, iRACINexome and iTRACINexome was prepared as in example 1 except that we took only DNA elements and parts of DNA elements overlapping with reference exome.

### Monitoring of Metastasis Free Survival

Metastasis free survival of patients in Group 1 (G.1) and in Group 2 (G.2) was monitored. For each Group, Patients were further divided according to administration of chemotherapy or absence of chemotherapy and difference in metastasis free survival was reported as Kaplan-Meier Curve.

### Results

Both iTRACexome and iRACINexome Stratified Chemotherapeutic Response in LMS

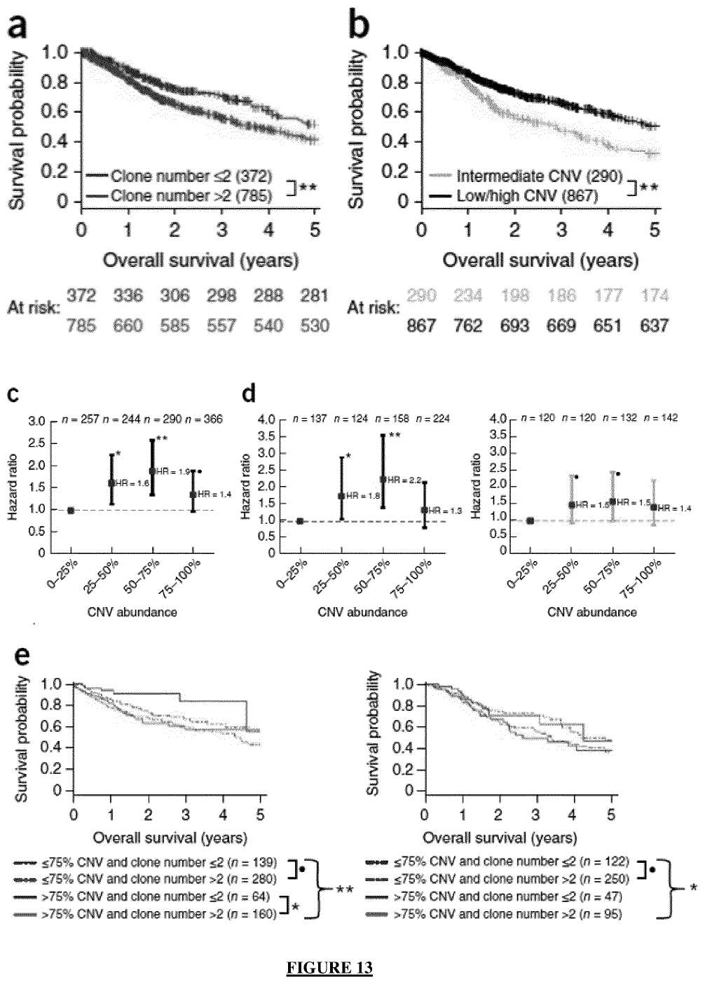

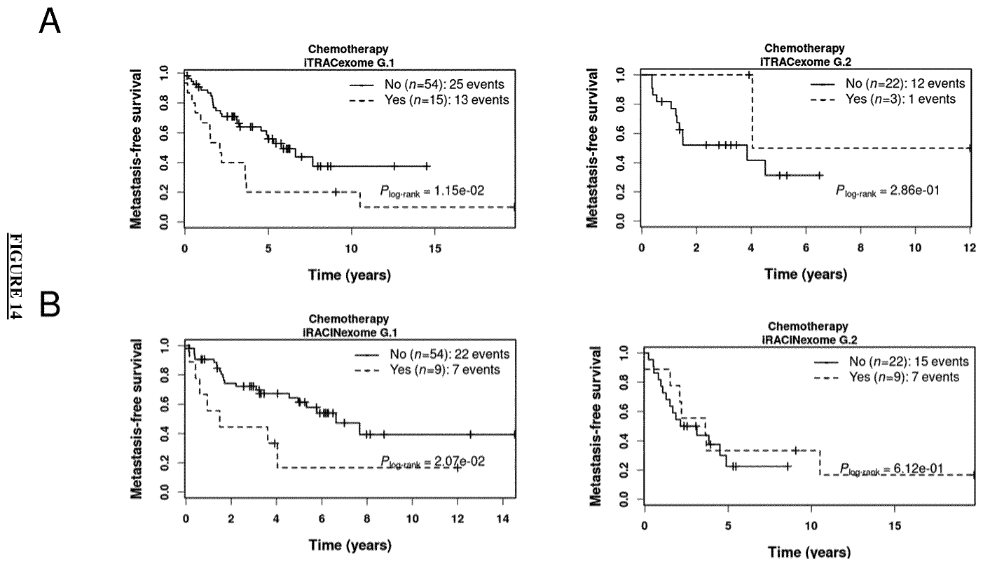

iTRACexome and iRACINexome are quantitative biomarkers and a threshold is required to split patients into groups having the biomarker and not having it. They are a new generation of biomarkers “statistical biomarkers” which values are immediately transferable to a level of confidence (pvalue/probabiliy) for having that biomarker. A patient having a score of 3 in any biomarker based on Hscore correspond to a probability of 0.001 that at least the observed number of breakpoints or mutations could be due random events (chance). The more unlikely the observed breakpoints in the biomarker's DNA elements are due to random events the more likely they are due structural and/or functional properties of the biomarker's DNA elements in the tested tumor sample. In hypothesis testing, a commonly used arbitrary threshold of 0.05 is used for significance level. Here we used a threshold of 0.001 corresponding to an Hscore of 3. using that threshold and for each biomarker, we split the LMS cohort into 2 groups: Group G.1 not having the biomarker (Hscore <=3) and Group G.2 having the biomarker (Hscore >3). We then tested chemotherapeutic response in each group. We found that in Group G.1 of both iTRACexome and iRACINexome, there is a significant difference in metastasis free survival (MFS) between patients receiving chemotherapy (Yes) and not receiving it (No) (p=) (FIG. 14 A left, FIG. 14 B left), while in Group G.2 there is no significant difference (FIG. 14 A right, FIG. 14 B right).

Combination of iTRACexome and iRACINexome Groups into MAGIC.2 Classifier or into a Single Biomarker Stratifies More Significantly Chemotherapeutic Response in LMS.

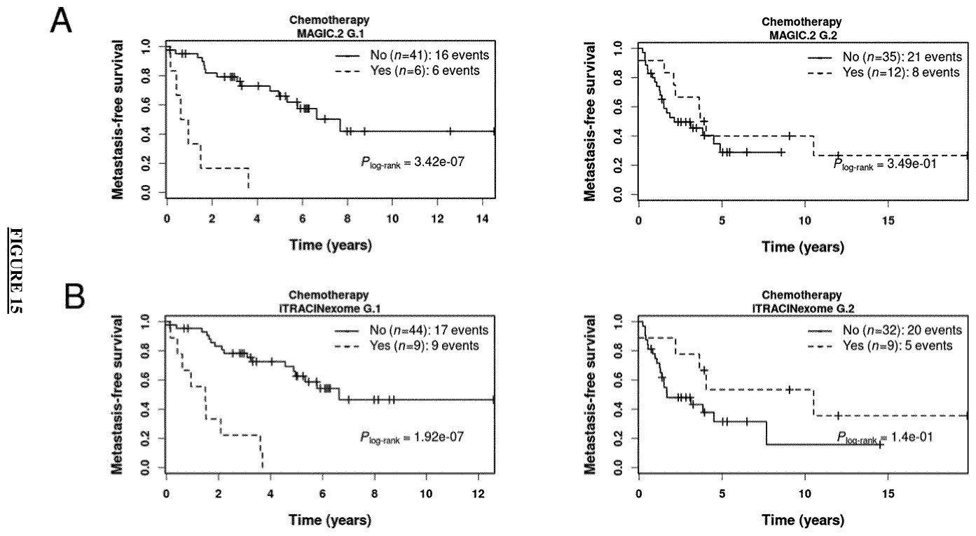

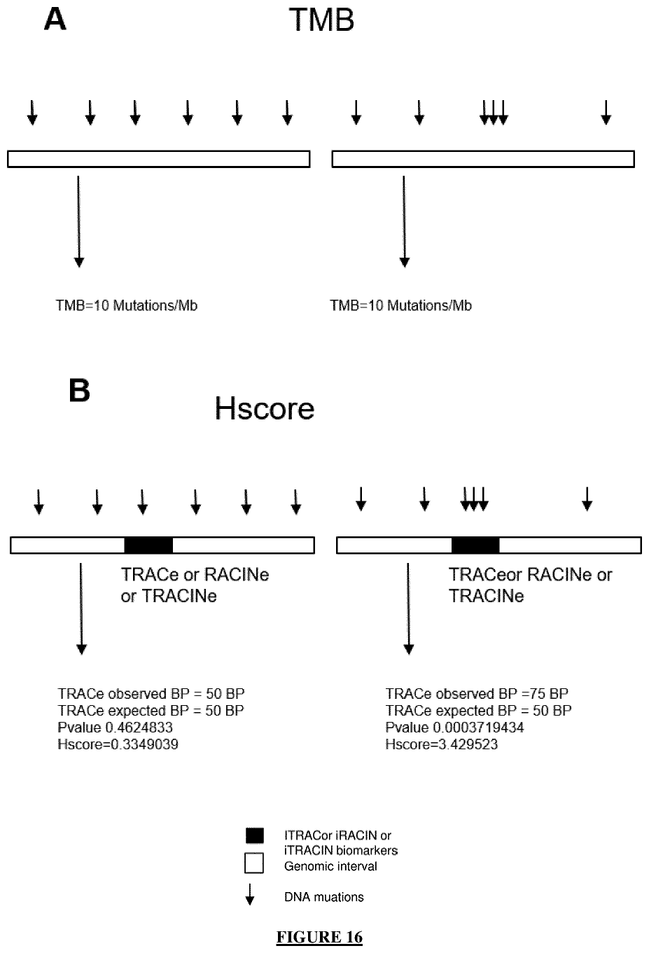

MAGIC.2 Group G.1 is defined as patients not having any of iTRACexome or iRACINexome biomarker, while Group G.2 is composed of patients having at least one of them or both of them. iTRACINexome stands for transcription and replication associated chromosomal instability index in exome. iTRACINexome is the combination at DNA sequence level of iTRACexome and iRACINexome. Then Hscore is computed as in example 1. We found that Group G.1 of both MAGIC.2 and iTRACINexome give similar results than iTRACexome and iRACINexome but much more significant FIG. 15 A left and FIG. 15 B left. Again G.2 patients have no significant difference in chemotherapeutic response between chemotherapy group (Yes) and control no chemotherapy (No) FIG. 15 A right and FIG. 15 B right. We expect that with the increased number of the included patients that the G.2 Group will have a therapeutic benefice from chemotherapy as there was a slight non-significant yet consistent tendency that chemotherapy group (Yes) having better MFS than control no chemotherapy (No) using iTRACexome, iRACINexome as well as iTRACINexome and MAGIC.2 FIG. 14 and FIG. 15.

Hscore is Different from Tumor Mutational Burden (TMB).

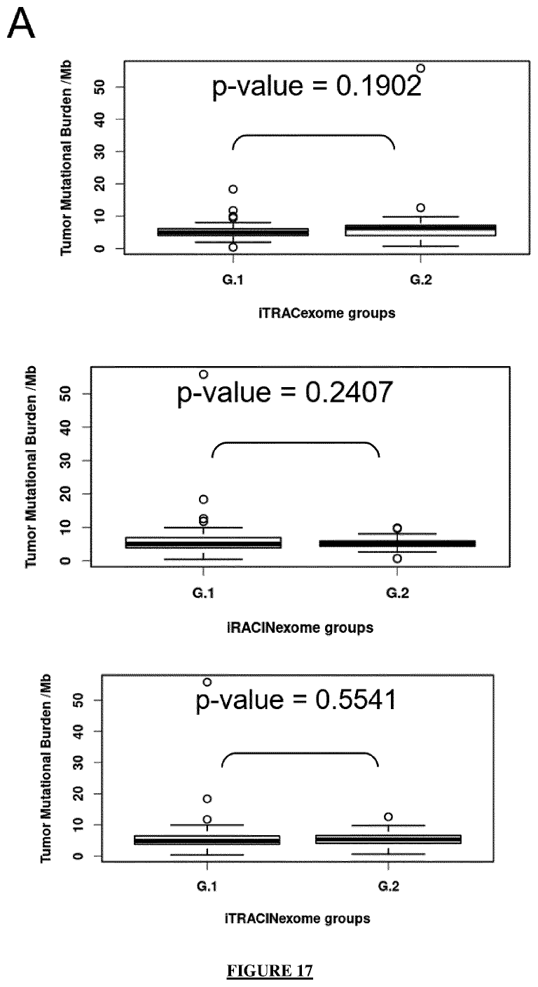

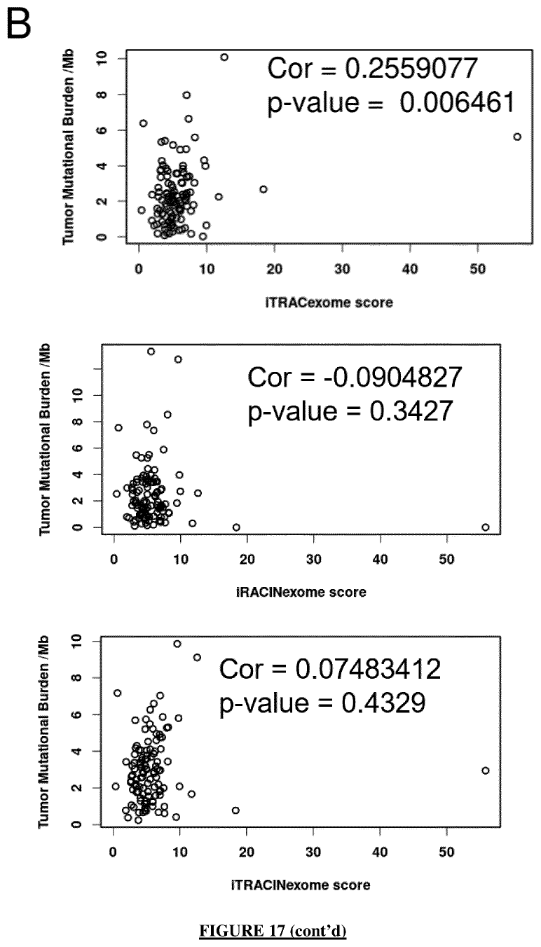

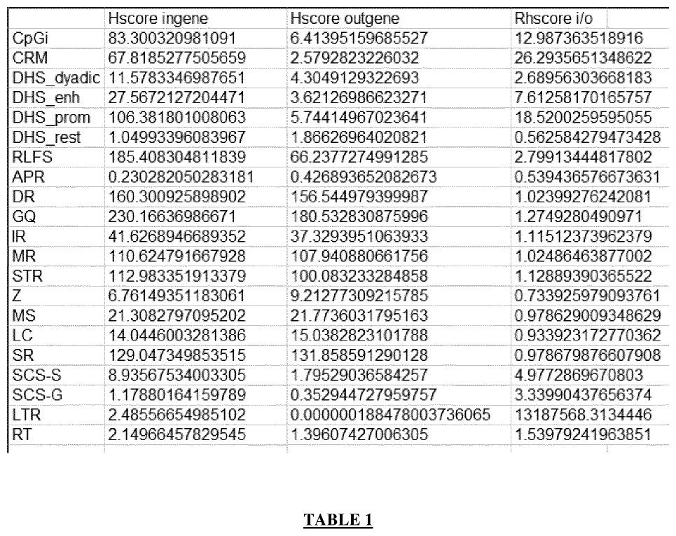

TMB is defined as the number of somatic mutations per megabase of interrogated genomic sequence (Merino et 2020). Hscore is defined as −log 10 the probability of obtaining at least as observed mutations in a sub-interval of the interrogated genomic sequence based on a random breakage model mutation distribution (Benhaddou et al 2021). While TMB is a measure of mutations density, Hscore methodology is a measure of mutations distribution. FIG. 16 demonstrates that TMB give the same value on a given genomic interval irrespective of BP distribution while Hscore give different values accounting for differences in mutations distribution inside the interrogated genomic interval. Furthermore, FIG. 17 shows that TMB and our iTRACexome, iRACINexome and iTRACINexome biomarkers, which are based on Hscore, are poorly or not correlated at all. Finally, we show that groups G.1 and G.2 of all our biomarkers do not have any difference in TMB further stressing the fact that Hscore and TMB are not the same biomarkers neither measure the same tumor's mutations properties.

Those results show that the exome may be used as tumor genomic DNA sequence to identify, determine genomic positions, and quantify the total quantity of a predefined marker of DNA structure or function alteration.

Also, the data shows that the indexes iTRAC (and iTRACexome), iRACIN (and iRACINexome) and the combination of them iTRACIN, (and iTRACINexome) are all efficient to stratify cancer patients according to the Hscore in groups for which chemotherapy may have or not a therapeutic benefice in terms of metastasis free survival.

iTRAC, iRACIN, and the combination iTRACIN biomarkers, which are computed based on Hscore methodology, may be used as biomarkers to predict the therapeutic benefit of chemotherapy treatment in a cancer patient.

Hscore is different from TMB methodologically and conceptually and is not correlated to it.

### REFERENCES

Abkevich, V, K M Timms, B T Hennessy, J Potter, M S Carey, L A Meyer, K Smith-McCune, et al. 2012. «Patterns of Genomic Loss of Heterozygosity Predict Homologous Recombination Repair Defects in Epithelial Ovarian Cancer». British Journal of Cancer 107 (10): 1776-82. https://doi.org/10.1038/bjc.2012.451.
Aguilera, Andrés. 2002. «The Connection between Transcription and Genomic Instability». The EMBO Journal 21 (3): 195-201. https://doi.org/10.1093/emboj/21.3.195.
Ahmad, S. S., K. Ahmed, et A. R. Venkitaraman. 2018. «Science in Focus: Genomic Instability and Its Implications for Clinical Cancer Care». Clinical Oncology 30 (12): 751-55. https://doi.org/10.1016/j.clon.2018.09.001.
Andersson, Carola, Henrik Fagman, Magnus Hansson, et Fredrik Enlund. 2016. «Profiling of Potential Driver Mutations in Sarcomas by Targeted next Generation Sequencing». Cancer Genetics 209 (4): 154-60. https://doi.org/10.1016/j.cancergen.2016.02.004.
Andor, Noemi, Trevor A Graham, Marnix Jansen, Li C Xia, C Athena Aktipis, Claudia Petritsch, Hanlee P Ji, et Carlo C Maley. 2016. «Pan-Cancer Analysis of the Extent and Consequences of Intratumor Heterogeneity». Nature Medicine 22 (1): 105-13. https://doi.org/10.1038/nm.3984.
Azvolinsky, Anna, Paul G. Giresi, Jason D. Lieb, et Virginia A. Zakian. 2009. «Highly Transcribed RNA Polymerase II Genes Are Impediments to Replication Fork Progression in Saccharomyces Cerevisiae». Molecular Cell 34 (6): 722-34. https://doi.org/10.1016/j.molcel.2009.05.022.
Bakhoum, Samuel F., Lilian Kabeche, Matthew D. Wood, Christopher D. Laucius, Dian Qu, Ashley M. Laughney, Gloria E. Reynolds, et al. 2015. «Numerical Chromosomal Instability Mediates Susceptibility to Radiation Treatment». Nature Communications 6 (1): 5990. https://doi.org/10.1038/ncomms6990.
Baumbusch, Lars O., Åslaug Helland, Yun Wang, Knut Liestøl, Marci E. Schaner, Ruth Holm, Dariush Etemadmoghadam, et al. 2013. «High Levels of Genomic Aberrations in Serous Ovarian Cancers Are Associated with Better Survival». Édité par Alexander James Roy Bishop. PLoS ONE 8 (1): e54356. https://doi.org/10.1371/journal.pone.0054356.
Ben-Ami, Eytan, Constance M. Barysauskas, Sarah Solomon, Kadija Tahlil, Rita Malley, Melissa Hohos, Kathleen Polson, et al. 2017. «Immunotherapy with Single Agent Nivolumab for Advanced Leiomyosarcoma of the Uterus: Results of a Phase 2 Study». Cancer 123 (17): 3285-90. https://doi.org/10.1002/cncr.30738.
Benhaddou, A., Gaston, L., Pérot, G. et al. Medium levels of transcription and replication related chromosomal instability are associated with poor clinical outcome. Sci Rep 11, 23429 (2021). https://doi.org/10.1038/s41598-021-02787-x
Birkbak, Nicolai J., Zhigang C. Wang, Ji-Young Kim, Aron C. Eklund, Qiyuan Li, Ruiyang Tian, Christian Bowman-Colin, et al. 2012. «Telomeric Allelic Imbalance Indicates Defective DNA Repair and Sensitivity to DNA-Damaging Agents». Cancer Discovery 2 (4): 366-75. https://doi.org/10.1158/2159-8290.CD-11-0206.
Blay, J.-Y., P. Soibinet, N. Penel, E. Bompas, F. Duffaud, E. Stoeckle, O. Mir, et al. 2017. «Improved Survival Using Specialized Multidisciplinary Board in Sarcoma Patients». Annals of Oncology: Official Journal of the European Society for Medical Oncology 28 (11): 2852-59. https://doi.org/10.1093/annonc/mdx484.
Blin, Marion, Benoit Le Tallec, Viola Nahse, Mélanie Schmidt, Caroline Brossas, Gael A. Millot, Marie-Noëlle Prioleau, et Michelle Debatisse. 2019. «Transcription-Dependent Regulation of Replication Dynamics Modulates Genome Stability». Nature Structural & Molecular Biology 26 (1): 58-66. https://doi.org/10.1038/s41594-018-0170-1.
Cer, Regina Z., Duncan E. Donohue, Uma S. Mudunuri, Nuri A. Temiz, Michael A. Loss, Nathan J. Starner, Goran N. Halusa, et al. 2012. «Non-B DB v2.0: A Database of Predicted Non-B DNA-Forming Motifs and Its Associated Tools». Nucleic Acids Research 41 (D1): D94-100. https://doi.org/10.1093/nar/gks955.
Chèneby, Jeanne, Marius Gheorghe, Marie Artufel, Anthony Mathelier, et Benoit Ballester. 2018. «ReMap 2018: An Updated Atlas of Regulatory Regions from an Integrative Analysis of DNA-Binding ChIP-Seq Experiments». Nucleic Acids Research 46 (D1): D267-75. https://doi.org/10.1093/nar/gkx1092.
Chibon, Frédéric, Pauline Lagarde, Sébastien Salas, Gaëlle Pérot, Véronique Brouste, Franck Tirode, Carlo Lucchesi, et al. 2010. «Validated Prediction of Clinical Outcome in Sarcomas and Multiple Types of Cancer on the Basis of a Gene Expression Signature Related to Genome Complexity». Nature Medicine 16 (7): 781-87. https://doi.org/10.1038/nm.2174.
Chomczynski, P., et N. Sacchi. 1987. «Single-Step Method of RNA Isolation by Acid Guanidinium Thiocyanate-Phenol-Chloroform Extraction». Analytical Biochemistry 162 (1): 156-59. https://doi.org/10.1006/abio.1987.9999.
Chudasama, Priya, Sadaf S. Mughal, Mathijs A. Sanders, Daniel Hübschmann, Inn Chung, Katharina I. Deeg, Siao-Han Wong, et al. 2018. «Integrative Genomic and Transcriptomic Analysis of Leiomyosarcoma». Nature Communications 9 (1): 144. https://doi.org/10.1038/s41467-017-02602-0.
Coindre, J. M., P. Terrier, L. Guillou, V. Le Doussal, F. Collin, D. Ranchère, X. Sastre, M. O. Vilain, F. Bonichon, et B. N'Guyen Bui. 2001. «Predictive Value of Grade for Metastasis Development in the Main Histologic Types of Adult Soft Tissue Sarcomas: A Study of 1240 Patients from the French Federation of Cancer Centers Sarcoma Group». Cancer 91 (10): 1914-26. https://doi.org/10.1002/1097-0142(20010515)91:10<1914::aid-cncrl214>3.0.co;2-3.
Davoli, Teresa, Hajime Uno, Eric C. Wooten, et Stephen J. Elledge. 2017. «Tumor Aneuploidy Correlates with Markers of Immune Evasion and with Reduced Response to Immunotherapy». Science 355 (6322): eaaf8399. https://doi.org/10.1126/science.aaf8399.
Debatisse, Michelle, Benoit Le Tallec, Anne Letessier, Bernard Dutrillaux, et Olivier Brison. 2012. «Common Fragile Sites: Mechanisms of Instability Revisited». Trends in Genetics 28 (1): 22-32. https://doi.org/10.1016/j.tig.2011.10.003.
Derré, J., R. Lagacé, A. Nicolas, A. Mairal, F. Chibon, J. M. Coindre, P. Terrier, X. Sastre, et A. Aurias. 2001. «Leiomyosarcomas and Most Malignant Fibrous Histiocytomas Share Very Similar Comparative Genomic Hybridization Imbalances: An Analysis of a Series of 27 Leiomyosarcomas». Laboratory Investigation; a Journal of Technical Methods and Pathology 81 (2): 211-15. https://doi.org/10.1038/labinvest.3780229.
Deshpande, A. M., et C. S. Newlon. 1996. «DNA Replication Fork Pause Sites Dependent on Transcription». Science 272 (5264): 1030-33. https://doi.org/10.1126/science.272.5264.1030.
Fletcher, C., Bridge, J. A., Hogendoorn, P. & Mertens, F. WHO Classification of Tumours of Soft Tissue and Bone. vol. 5 (IARC Press, 2013).
French, S. 1992. «Consequences of Replication Fork Movement through Transcription Units in Vivo». Science 258 (5086): 1362-65. https://doi.org/10.1126/science.1455232.
Gaillard, Hélène, Emilia Herrera-Moyano, et Andrés Aguilera. 2013. «Transcription-Associated Genome Instability». Chemical Reviews 113 (11): 8638-61. https://doi.org/10.1021/cr400017y.
Gaillard, Hélène, et Andrés Aguilera. 2016. «Transcription as a Threat to Genome Integrity». Annual Review of Biochemistry 85 (1): 291-317. https://doi.org/10.1146/annurev-biochem-060815-014908.
Gaillard, Hélène, Tatiana García-Muse, et Andrés Aguilera. 2015. «Replication Stress and Cancer». Nature Reviews Cancer 15 (5): 276-89. https://doi.org/10.1038/nrc3916.
Gómez-Gonzalez, Belén, María García-Rubio, Rodrigo Bermejo, Hélène Gaillard, Katsuhiko Shirahige, Antonio Marin, Marco Foiani, et Andrés Aguilera. 2011. «Genome-wide function of THO/TREX in active genes prevents R-loop-dependent replication obstacles». The EMBO Journal 30 (15): 3106-19. https://doi.org/10.1038/emboj.2011.206.
Graaf, Winette T A van der, Jean-Yves Blay, Sant P Chawla, Dong-Wan Kim, Binh Bui-Nguyen, Paolo G Casali, Patrick Schöffski, et al. 2012. «Pazopanib for Metastatic Soft-Tissue Sarcoma (PALETTE): A Randomised, Double-Blind, Placebo-Controlled Phase 3 Trial». The Lancet 379 (9829): 1879-86. https://doi.org/10.1016/S0140-6736(12)60651-5.
Guillou, L, J M Coindre, F Bonichon, B B Nguyen, P Terrier, F Collin, M O Vilain, et al. 1997. «Comparative Study of the National Cancer Institute and French Federation of Cancer Centers Sarcoma Group Grading Systems in a Population of 410 Adult Patients with Soft Tissue Sarcoma.» Journal of Clinical Oncology 15 (1): 350-62. https://doi.org/10.1200/JCO.1997.15.1.350.
Helmrich, Anne, Monica Ballarino, et Laszlo Tora. 2011. «Collisions between Replication and Transcription Complexes Cause Common Fragile Site Instability at the Longest Human Genes». Molecular Cell 44 (6): 966-77. https://doi.org/10.1016/j.molcel.2011.10.013.
Janssen, Aniek, Geert J. P. L. Kops, et René H. Medema. 2009. «Elevating the Frequency of Chromosome Mis-Segregation as a Strategy to Kill Tumor Cells». Proceedings of the National Academy of Sciences 106 (45): 19108-13. https://doi.org/10.1073/pnas.0904343106.
Jenjaroenpun, Piroon, Thidathip Wongsurawat, Surya Pavan Yenamandra, et Vladimir A. Kuznetsov. 2015. «QmRLFS-Finder: A Model, Web Server and Stand-Alone Tool for Prediction and Analysis of R-Loop Forming Sequences». Nucleic Acids Research 43 (W1): W527-34. https://doi.org/10.1093/nar/gkv344.
Jinks-Robertson, Sue, et Ashok S. Bhagwat. 2014. «Transcription-Associated Mutagenesis». Annual Review of Genetics 48 (1): 341-59. https://doi.org/10.1146/annurev-genet-120213-092015.
Judson, Ian, Jaap Verweij, Hans Gelderblom, Jörg T. Hartmann, Patrick Schöffski, Jean-Yves Blay, J. Martijn Kerst, et al. 2014. «Doxorubicin Alone versus Intensified Doxorubicin plus Ifosfamide for First-Line Treatment of Advanced or Metastatic Soft-Tissue Sarcoma: A Randomised Controlled Phase 3 Trial». The Lancet. Oncology 15 (4): 415-23. https://doi.org/10.1016/S1470-2045(14)70063-4.
Kent, W. James. 2002. «BLAT—the BLAST-like Alignment Tool». Genome Research 12 (4): 656-64. https://doi.org/10.1101/gr.229202.
Kim, Nayun, et Sue Jinks-Robertson. 2012. «(Transcription as a Source of Genome Instability». Nature Reviews Genetics 13 (3): 204-14. https://doi.org/10.1038/nrg3152.
Kuhn, Robert M., David Haussler, et W. James Kent. 2013. «The UCSC Genome Browser and Associated Tools». Briefings in Bioinformatics 14 (2): 144-61. https://doi.org/10.1093/bib/bbs038.
Langmead, Ben, Cole Trapnell, Mihai Pop, et Steven L Salzberg. 2009. «Ultrafast and Memory-Efficient Alignment of Short DNA Sequences to the Human Genome». Genome Biology 10 (3): R25. https://doi.org/10.1186/gb-2009-10-3-r25.
Le Tallec, Benoit, Gael Armel Millot, Marion Esther Blin, Olivier Brison, Bernard Dutrillaux, et Michelle Debatisse. 2013. «Common Fragile Site Profiling in Epithelial and Erythroid Cells Reveals That Most Recurrent Cancer Deletions Lie in Fragile Sites Hosting Large Genes». Cell Reports 4 (3): 420-28. https://doi.org/10.1016/j.celrep.2013.07.003.
Li, H., et R. Durbin. 2009. «Fast and Accurate Short Read Alignment with Burrows-Wheeler Transform». Bioinformatics 25 (14): 1754-60. https://doi.org/10.1093/bioinformatics/btp324.
Li, Heng, Bob Handsaker, Alec Wysoker, Tim Fennell, Jue Ruan, Nils Homer, Gabor Marth, Goncalo Abecasis, Richard Durbin, et 1000 Genome Project Data Processing Subgroup. 2009. «The Sequence Alignment/Map Format and SAMtools». Bioinformatics (Oxford, England) 25 (16): 2078-79.
Limpert, Eckhard, Werner A. Stahel, et Markus Abbt. 2001. «Log-Normal Distributions across the Sciences: Keys and Clues». BioScience 51 (5): 341.
Macheret, Morgane, et Thanos D. Halazonetis. 2015. «DNA Replication Stress as a Hallmark of Cancer». Annual Review of Pathology: Mechanisms of Disease 10 (1): 425-48. https://doi.org/10.1146/annurev-pathol-012414-040424.
Madireddy, Advaitha, Settapong T. Kosiyatrakul, Rebecca A. Boisvert, Emilia Herrera-Moyano, María L. García-Rubio, Jeannine Gerhardt, Elizabeth A. Vuono, et al. 2016. «FANCD2 Facilitates Replication through Common Fragile Sites». Molecular Cell 64 (2): 388-404. https://doi.org/10.1016/j.molcel.2016.09.017.
Marnef, Aline, Sarah Cohen, et Gaëlle Legube. 2017. «Transcription-Coupled DNA Double-Strand Break Repair: Active Genes Need Special Care». Journal of Molecular Biology 429 (9): 1277-88.
Merino D M, McShane L M, Fabrizio D, Funari V, Chen S J, White J R, Wenz P, Baden J, Barrett J C, Chaudhary R, Chen L, Chen W S, Cheng J H, Cyanam D, Dickey J S, Gupta V, Hellmann M, Helman E, Li Y, Maas J, Papin A, Patidar R, Quinn K J, Rizvi N, Tae H, Ward C, Xie M, Zehir A, Zhao C, Dietel M, Stenzinger A, Stewart M, Allen J; TMB Harmonization Consortium. Establishing guidelines to harmonize tumor mutational burden (TMB): in silico assessment of variation in TMB quantification across diagnostic platforms: phase I of the Friends of Cancer Research TMB Harmonization Project. J Immunother Cancer. 2020 March; 8(1):e000147. doi: 10.1136/jitc-2019-000147. PMID: 32217756; PMCID: PMC7174078.
Mirza, Mansoor R., Bradley J. Monk, Jørn Herrstedt, Amit M. Oza, Sven Mahner, Andrés Redondo, Michel Fabbro, et al. 2016. «Niraparib Maintenance Therapy in Platinum-Sensitive, Recurrent Ovarian Cancer». New England Journal of Medicine 375 (22): 2154-64. https://doi.org/10.1056/NEJMoa1611310.
Morgan, Graeme, Robyn Ward, et Michael Barton. 2004. «The Contribution of Cytotoxic Chemotherapy to 5-Year Survival in Adult Malignancies». Clinical Oncology 16 (8): 549-60. https://doi.org/10.1016/j.clon.2004.06.007.
Mourad, Raphaël, Krzysztof Ginalski, Gaëlle Legube, et Olivier Cuvier. 2018. «Predicting Double-Strand DNA Breaks Using Epigenome Marks or DNA at Kilobase Resolution». Genome Biology 19 (1): 34. https://doi.org/10.1186/si3059-018-1411-7.
Negrini, Simona, Vassilis G. Gorgoulis, et Thanos D. Halazonetis. 2010. «Genomic Instability an Evolving Hallmark of Cancer». Nature Reviews Molecular Cell Biology 11 (3): 220-28. https://doi.org/10.1038/nrm2858.
Pentzold, Constanze, Shiraz Ali Shah, Niels Richard Hansen, Benoit Le Tallec, Andaine Seguin-Orlando, Michelle Debatisse, Michael Lisby, et Vibe H Oestergaard. 2018. «FANCD2 Binding Identifies Conserved Fragile Sites at Large Transcribed Genes in Avian Cells». Nucleic Acids Research 46 (3): 1280-94. https://doi.org/10.1093/nar/gkxl260.
Pérot, Gaëlle, Frëdëric Chibon, Audrey Montero, Pauline Lagarde, Hugues de Thë, Philippe Terrier, Louis Guillou, Dominique Ranchère, Jean-Michel Coindre, et Alain Aurias. 2010. «Constant P53 Pathway Inactivation in a Large Series of Soft Tissue Sarcomas with Complex Genetics». The American Journal of Pathology 177 (4): 2080-90. https://doi.org/10.2353/ajpath.2010.100104.
Popova, T., E. Manie, G. Rieunier, V. Caux-Moncoutier, C. Tirapo, T. Dubois, O. Delattre, et al. 2012. «Ploidy and Large-Scale Genomic Instability Consistently Identify Basal-like Breast Carcinomas with BRCA1/2 Inactivation». Cancer Research 72 (21): 5454-62. https://doi.org/10.1158/0008-5472.CAN-12-1470.
Roadmap Epigenomics Consortium, Anshul Kundaje, Wouter Meuleman, Jason Ernst, Misha Bilenky, Angela Yen, Alireza Heravi-Moussavi, et al. 2015. «Integrative Analysis of 111 Reference Human Epigenomes». Nature 518 (7539): 317-30. https://doi.org/10.1038/nature14248.
Sansregret, Laurent, Bart Vanhaesebroeck, et Charles Swanton. 2018. «Determinants and Clinical Implications of Chromosomal Instability in Cancer». Nature Reviews Clinical Oncology 15 (3): 139-50. https://doi.org/10.1038/nrclinonc.2017.198.
Schneider, Valerie A., Tina Graves-Lindsay, Kerstin Howe, Nathan Bouk, Hsiu-Chuan Chen, Paul A. Kitts, Terence D. Murphy, et al. 2017. «Evaluation of GRCh38 and de Novo Haploid Genome Assemblies Demonstrates the Enduring Quality of the Reference Assembly». Genome Research 27 (5): 849-64. https://doi.org/10.1101/gr.213611.116.
Shabani Azim, Faezeh, Hamidreza Houri, Zohreh Ghalavand, et Bahram Nikmanesh. 2018. «Next Generation Sequencing in Clinical Oncology: Applications, Challenges and Promises: A Review Article». Iranian Journal of Public Health 47 (10): 1453-57.
Spencer, David H., Jennifer K. Sehn, Haley J. Abel, Mark A. Watson, John D. Pfeifer, et Eric J. Duncavage. 2013. «Comparison of Clinical Targeted Next-Generation Sequence Data from Formalin-Fixed and Fresh-Frozen Tissue Specimens». The Journal of Molecular Diagnostics 15 (5): 623-33. https://doi.org/10.1016/j.jmoldx.2013.05.004.
Stefansson, Olafur Andri, Jon Gunnlaugur Jonasson, Oskar Thor Johannsson, Kristrun Olafsdottir, Margret Steinarsdottir, Sigridur Valgeirsdottir, et Jorunn Erla Eyfjord. 2009. «Genomic Profiling of Breast Tumors in Relation to BRCAabnormalities and Phenotypes». Breast Cancer Research 11 (4): R47. https://doi.org/10.1186/bcr2334.
Técher, Hervé, Stéphane Koundrioukoff, Alain Nicolas, et Michelle Debatisse. 2017. «The Impact of Replication Stress on Replication Dynamics and DNA Damage in Vertebrate Cells». Nature Reviews Genetics 18 (9): 535-50. https://doi.org/10.1038/nrg.2017.46.
Tubbs, Anthony, et André Nussenzweig. 2017. «Endogenous DNA Damage as a Source of Genomic Instability in Cancer». Cell 168 (4): 644-56.
Van der Auwera G A & O'Connor B D. (2020). Genomics in the Cloud: Using Docker, GATK, and WDL in Terra (1st Edition). O'Reilly Media.
Wang, Guliang, et Karen M. Vasquez. 2014. «Impact of Alternative DNA Structures on DNA Damage, DNA Repair, and Genetic Instability». DNA Repair 19 (juillet): 143-51. https://doi.org/10.1016/j.dnarep.2014.03.017.
Wilson, Thomas E., Martin F. Arlt, So Hae Park, Sountharia Rajendran, Michelle Paulsen, Mats Ljungman, et Thomas W. Glover. 2015. «Large Transcription Units Unify Copy Number Variants and Common Fragile Sites Arising under Replication Stress». Genome Research 25 (2): 189-200. https://doi.org/10.1101/gr.177121.114.
Zasadil, L. M., K. A. Andersen, D. Yeum, G. B. Rocque, L. G. Wilke, A. J. Tevaarwerk, R. T. Raines, M. E. Burkard, et B. A. Weaver. 2014. «Cytotoxicity of Paclitaxel in Breast Cancer Is Due to Chromosome Missegregation on Multipolar Spindles». Science Translational Medicine 6 (229): 229ra43-229ra43. https://doi.org/10.1126/scitranslmed.3007965.
Zhang, Shaojun, Yuan Yuan, et Dapeng Hao. 2014. «A Genomic Instability Score in Discriminating Nonequivalent Outcomes of BRCA1/2 Mutations and in Predicting Outcomes of Ovarian Cancer Treated with Platinum-Based Chemotherapy». Édité par Gautam Chaudhuri. PLoS ONE 9 (12): e113169. https://doi.org/10.1371/journal.pone.0113169.
Zhou, Weichen, Feng Zhang, Xiaoli Chen, Yiping Shen, James R. Lupski, et Li Jin. 2013. «(Increased Genome Instability in Human DNA Segments with Self-Chains: Homology-Induced Structural Variations via Replicative Mechanisms». Human Molecular Genetics 22 (13): 2642-51. https://doi.org/10.1093/hmg/ddt113.

1. A method for classifying a patient having a cancer in a group representative of: an evolution of said cancer and/or a risk of metastasis of said cancer and/or a sensibility to a treatment for the cancer, and then treating the patient, the method using:
i) a tumor genomic DNA sequence obtained by sequencing a tumor genomic DNA obtained from said patient,
ii) at least one set of preselected DNA elements, each DNA element having a genomic position, said set of DNA elements having a length L in base pairs,
iii) a preselected genomic DNA sequence of reference having a length l in base pairs, and
iv) at least one reference threshold separating two groups of patients, each group being representative of a different evolution of said cancer,
said method comprising at least the steps of:
a) identifying, determining genomic positions, and quantifying a total quantity n of a predefined marker of DNA structure alteration within said tumor genomic DNA sequence of the tumor genomic DNA from said patient, by comparing said tumor genomic DNA sequence with said preselected genomic DNA sequence of reference,
b) associating, to the set of preselected DNA elements, a quantity N of the predefined marker of DNA structure alteration, from the total quantity n, according to the genomic positions of the DNA elements of the set of preselected DNA elements and to the genomic positions of said predefined markers of DNA structure alteration obtained at step a),
c) computing a test score relative to a quantity N of predefined markers of DNA structure alteration associated with the length L of said set of preselected DNA elements, said test score being based on a comparison between said quantity N of predefined markers of DNA structure alteration and a reference level defined by a random model relative to the total quantity n of the predefined marker of DNA structure alteration over the length l of the preselected genomic DNA sequence of reference,
d) comparing said test score to said at least one reference threshold in order to classify said patient in the group representative of: the evolution of said cancer, and/or the risk of metastasis of said cancer and/or sensibility to a treatment for the cancer, and
e) treating said patient with at least one cancer treatment associated with the group.

2. The method according to claim 1, wherein the method further comprises the use of a non-tumor genomic DNA sequence obtained by sequencing a genomic DNA obtained from non-cancer cells of said patient.

3. The method according to claim 2, wherein step a) comprises aligning and comparing the tumor genomic DNA sequence, the non-tumor genomic DNA, and the preselected genomic DNA sequence of reference.

4. The method according to claim 1, wherein the predefined marker of DNA structure alteration is selected in from the group consisting of breakpoint, insertion, deletion, mutation, duplication, inversion, translocation, complex genomic rearrangements, copy number variation, telomeric allelic imbalance, large-scale state transitions, and loss of heterozygosity.

5. The method according to claim 1, wherein said DNA elements are selected from the group consisting of direct repeats (DR), short tandem repeats (STR), mirror repeats (MR), inverted repeats (IR), Z DNA, simple repeats (SR), microsatellites (MS), low complexities (LC), R-loops forming sequences (RLFS), G-quadruplexes (GQ), CpG islands (CpGi), cis-regulated modules (CRM), self-chains segments self-aligned (SCS-S), DNase I hypersensitivity site (DHS) promoters (DHS_prom), DNase I hypersensitivity site (DHS) enhancers (DHS_enh), DNase I hypersensitivity site (DHS) dyadic regulatory elements (DHS_dyadic), DNase I hypersensitivity site (DHS) of type rest (DHS_rest), transcription factor binding sites, and mixtures thereof.

6. The method according to claim 1, wherein said set of preselected of DNA elements comprises replication-associated chromosomal instability elements selected from the group consisting of direct repeats (DR), short tandem repeats (STR), mirror repeats (MR), inverted repeats (IR), Z DNA, simple repeats (SR), microsatellites (MS), and low complexities (LC), and/or comprises transcription-associated chromosomal instability elements selected from the group consisting of R-loops forming sequences (RLFS), G-quadruplexes (GQ), CpG islands (CpGi), cis-regulated modules (CRM), self-chains segments self-aligned (SCS-S), DNase I hypersensitivity site (DHS) promoters (DHS_prom), DNase I hypersensitivity site (DHS) enhancers (DHS_enh), DNase I hypersensitivity site (DHS) dyadic regulatory elements (DHS_dyadic), DNase I hypersensitivity site (DHS) of type rest (DHS_rest), transcription factor binding sites, and mixtures thereof.

7. The method according to claim 1, wherein the predefined marker of DNA structure alteration is a breakpoint, and wherein a quantity of the predefined marker of DNA structure alteration is associated with each DNA element as a quantity N₁, N₂, N₃, . . . of the predefined marker of DNA structure alteration, from the total quantity n, said association being according to the genomic positions of said DNA element and to the genomic positions of the predefined markers of DNA structure alteration.

8. The method according to claim 1, wherein in step c), the test score uses a probability p of having said quantity N of predefined markers of DNA structure alteration associated with the length L of said set of preselected DNA elements, said probability p being computed with said random model defining a reference level and using a probability pᵤ relative to an isotropic distribution of the total quantity n of the predefined marker of DNA structure alteration over the length l of the preselected genomic DNA sequence of reference.

9. The method according to claim 8, wherein in step c), the test score uses a probability p of having said quantity N of predefined markers of DNA alteration within the length L of the set of preselected DNA elements, and the probability pᵤ is a ratio of the total quantity n of identified and quantified predefined markers of DNA structure alteration to the length l of the preselected genomic DNA sequence of reference.

10. The method according to claim 8, wherein, at step c), said probability p of having said quantity of predefined markers of DNA structure alteration associated with the length L of said set of preselected DNA elements is a one-sided p-value wherein only the values superior to said quantity of predefined markers of DNA structure alteration are taken into account, and said test score is computed as a logarithmic transformation of said p-value.

11. A diagnostic method for predicting an evolution of a cancer in a patient having said cancer, including predicting one or more of (i) a risk of metastasis of said cancer, (ii) a sensibility of said cancer to a cancer treatment, or (iii) a risk of mortality of said patient to said cancer, said method:
obtaining at least one tumor genomic DNA sequence by sequencing a tumor genomic DNA obtained from said patient, and
submitting said at least one tumor genomic DNA sequence to the method according to claim 1 for classifying the cancer patient in a group representative of an evolution of said cancer, said classification being predictive of an evolution of said cancer of said cancer patient.

12. A diagnostic method for identifying a genome instability mechanism of a cancer in a patient having said cancer, said method comprising:
obtaining at least one tumor genomic DNA sequence by sequencing a tumor genomic DNA obtained from said patient, and
submitting said at least one tumor genomic DNA sequence to the method according to claim 1 for classifying the cancer patient in a group representative of a genome instability mechanism being either a replication-associated chromosomal instability or a transcription-associated chromosomal instability.

13. A method for selecting a treatment of a cancer for a patient having said cancer and for treating said patient, said method comprising:
obtaining at least one tumor genomic DNA sequence by sequencing a tumor genomic DNA obtained from said patient,
submitting said at least one tumor genomic DNA sequence to the method according to claim 1 for classifying said cancer of said patient in a group associating said cancer with at least one cancer treatment, and
treating said patient with said at least one cancer treatment associated with said group.

14. A method for predicting a sensibility of a cancer in a patient having said cancer to a cancer treatment and for treating said patient, said method comprising:
obtaining at least one tumor genomic DNA sequence by sequencing a tumor genomic DNA obtained from said patient,
submitting said at least one tumor genomic DNA sequence to the method according to claim 1 for classifying said cancer of said patient in a group representative of a sensibility of said cancer to a cancer treatment, said classification being predictive of a sensibility of said cancer of said patient to said cancer treatment, and
treating said patient with said cancer treatment.

15. The method according to claim 1, wherein said cancer is selected from the group consisting of leukemias, lymphomas, carcinomas, melanomas, sarcomas, undifferentiated pleomorphic sarcoma, a liposarcoma, a rhabdomyosarcoma, an angiosarcoma from blood vessels, a malignant peripheral nerve sheath tumor (MPNST or PNST), a gastrointestinal stromal tumor sarcoma (GIST), a synovial sarcoma, a dermatofibrosarcoma, a fibrohistiocytic sarcoma, a myxofibrosarcoma, a Kaposi sarcoma, a chondro-osseous sarcomas, or a leiomyosarcoma.

16. The method of claim 1, wherein the at least one reference threshold is representative of an evolution of a cancer and/or a risk of metastasis for patients having said cancer, and/or sensibility to a treatment for the cancer and is determined using:
i) a classifier,
ii) a plurality of tumor genomic DNA sequences obtained by sequencing a plurality of tumor genomic DNA obtained from a plurality of patients having said cancer,
iii) at least one set of preselected DNA elements, each DNA element having a genomic position, said at least one set of preselected DNA elements having a length L in base pairs,
iv) a preselected genomic DNA sequence of reference having a length l in base pairs, and
v) a plurality of scores relative to predefined markers of DNA structure alteration associated with the length L of said at least one set of preselected DNA elements,
said predefined markers of DNA structure alteration being previously quantified and their genomic positions identified within each tumor genomic DNA sequence of the tumor genomic DNA from each patient, by comparing said tumor genomic DNA sequence with said preselected genomic DNA sequence of reference, to obtain for each tumor genomic DNA sequence a total quantity n of predefined markers of DNA structure alteration for each genomic DNA sequence,
said plurality of scores being based on a comparison between each total quantity n of predefined markers of DNA structure alteration and a reference level defined by a random model relative to each total quantity n of the predefined marker of DNA structure alteration over the length l of the preselected genomic DNA sequence of reference,
said method comprising at least the steps of:
a) choosing, based on said plurality of scores, predefined thresholds relative to said quantified predefined marker associated with the length L of said at least one set of preselected DNA elements, each of said predefined thresholds separating two groups representative of a different evolution of said cancer,
b) for each of said predefined thresholds, computing with the classifier, by using an estimation method, a comparison value between two groups defined by a corresponding threshold, and
c) determining at least one reference threshold as separating two groups each representative of a different evolution of said cancer by choosing a predefined threshold that has given an extremum value of each of said comparison values computed at step b).

17. A method for selecting a DNA element as a biomarker of an evolution of a cancer in a patient having said cancer, the method using:
i) a tumor genomic DNA sequence obtained by sequencing a tumor genomic DNA obtained from said patient,
ii) a plurality of preselected DNA elements, each DNA element having a genomic position, a length le in base pairs,
iii) a preselected genomic DNA sequence of reference having a length l in base pairs, and
iv) a preselected threshold or a set of preselected thresholds for selecting said DNA element to be used as a biomarker,
said method comprising at least the steps of:
a) identifying, determining genomic positions, and quantifying a total quantity n of a predefined marker of DNA structure alteration within said tumor genomic DNA sequence of the tumor genomic DNA from said patient, by comparing said tumor genomic DNA sequence with said preselected genomic DNA sequence of reference,
b) associating, to each DNA element of the plurality of DNA elements, a quantity Ne of the predefined marker of DNA structure alteration, from the total quantity n, according to the genomic positions of said DNA element and to the genomic positions of said predefined markers of DNA structure alteration obtained at step b),
c) computing for each DNA element a test score relative to said quantity Ne of predefined markers of DNA structure alteration associated with the length le of said DNA element, said test score being based on a comparison between said quantity Ne of predefined markers of DNA structure alteration and a reference level defined by random model relative to the total quantity n of the predefined marker of DNA structure alteration over the length l of the preselected genomic DNA sequence of reference, and
d) comparing said test scores to said preselected threshold or said set of preselected thresholds to select said DNA element as a biomarker of an evolution of a cancer in a patient having said cancer.

18. A biomarker of an evolution of a cancer in a patient having said cancer, said biomarker comprising at least one replication-associated chromosomal instability elements selected from the group consisting of direct repeats (DR), short tandem repeats (STR), mirror repeats (MR), inverted repeats (IR), Z DNA, simple repeats (SR), microsatellites (MS), and low complexities (LC).

19. A biomarker of an evolution of a cancer in a patient having said cancer, said biomarker comprising at least one transcription-associated chromosomal instability elements selected from the group consisting of R-loops forming sequences (RLFS), G-quadruplexes (GQ), CpG islands (CpGi), cis-regulated modules (CRM), self-chains segments self-aligned (SCS-S), DNase I hypersensitivity site (DHS) promoters (DHS_prom), DNase I hypersensitivity site (DHS) enhancers (DHS_enh), DNase I hypersensitivity site (DHS) dyadic regulatory elements (DHS_dyadic), DNase I hypersensitivity site (DHS) of type rest (DHS_rest), transcription factor binding sites, and mixtures thereof.

20. Computer program product for classifying a patient having a cancer in a group representative of an evolution of said cancer and a risk of metastasis of said cancer, using:
i) a tumor genomic DNA sequence obtained by sequencing a tumor genomic DNA obtained from isolated cancer cells of said patient,
ii) at least one set of preselected DNA elements, each DNA element having a genomic position, said preselected set of DNA elements having a length L in base pairs,
iii) a preselected genomic DNA sequence of reference having a length l in base pairs, and
iv) at least one reference threshold separating two groups of patients, each group being representative of a different evolution of said cancer,
the computer program product comprising a support and stored on this support instructions that can be read by a processor, these instructions being configured to:
a) identify, determine genomic positions, and quantify a total quantity n of predefined markers of DNA structure alteration within said tumor genomic DNA sequence of the tumor genomic DNA from said patient, by comparing said tumor genomic DNA sequence with said preselected genomic DNA sequence of reference,
b) associate, to the set of preselected DNA elements, a quantity of the predefined markers of DNA structure alteration according to the genomic positions of the DNA elements of the set of preselected DNA elements and to the genomic positions of said predefined markers of DNA structure alteration obtained at step a),
c) compute a test score relative to a quantity of predefined markers of DNA structure alteration associated with the length L of said set of preselected DNA elements, said test score being based on a comparison between said quantity of predefined markers of DNA structure alteration and a reference level defined by a random model relative to the total quantity n of the predefined marker of DNA structure alteration over the length l of the preselected genomic DNA sequence of reference, and
d) compare said test score to said at least one reference threshold in order to classify said patient.
